(19) United States
(12) Patent Application Publication
(10) Pub. No.: US 2024/0342295 A1
(43) Pub. Date: Oct. 17, 2024

(54) FUNCTIONALISATION OF CARBOXYMETHYLCELLULOSE

(71) Applicant: The University of Manchester, Manchester (GB)

(72) Inventors: Simon Webb, Manchester (GB); Jack Yung, Manchester (GB)

(21) Appl. No.: 18/292,634

(22) PCT Filed: Jul. 28, 2022
(86) PCT No.: PCT/EP2022/071262

§ 371 Date: Jan. 26, 2024

(30) Foreign Application Priority Data
Jul. 28, 2021 (GB) 2110867.5

(51) Int. Cl. A61K 47/61; A61K 47/62; A61K 49/00

(52) U.S. Cl. CPC A61K 47/61; A61K 47/62; A61K 49/0054

(57) ABSTRACT

The disclosure provides a method of functionalising sodium carboxy methylcellulose (Na-CMC) which is a cellulose comprising at least one unit of Formula (0), where at least one G⁰ group is (I) and the remaining G⁰ groups are H. The disclosure also provides a CMC derivative product obtainable by this method, wherein the CMC derivative product comprises at least one unit of Formula (3), wherein: L is (II), n is an integer from 0 to 450; X is selected from (III), (IV), (V), (VI), (VII), (VIII), and (IX); L¹ is a bond, alkylene, -(alkylene)O—*, —O(alkylene)-*, or -(alkylene)C(O)—*; wherein the*is the attachment to the cyclooctene or benzene ring; Z is —F, —Cl, —Br, or —I; R² is —L²-M; L² is an alkylene, -(alkylene)C(O)—*, -(alkylene)NHC(O)—*, —(CH₂CH₂O)ₘCH₂—*, —CH₂(OCH₂CH₂)ₘ—*, arylene, or a bond, wherein the*is the attachment to M and m is an integer between 1 to 450; and M is a biomolecular unit.

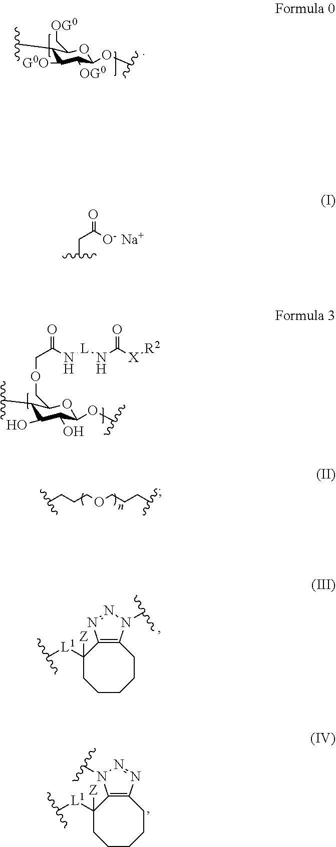

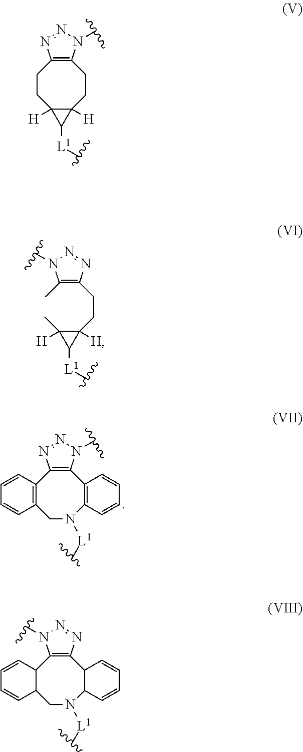

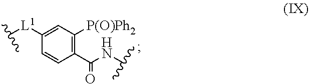

### FIELD OF THE INVENTION

The present invention relates to the functionalisation of carboxymethylcellulose (CMC) and particularly, although not exclusively, functionalisation of the sodium salt of CMC (Na-CMC) by high-throughput chemical modification to produce bioresponsive CMC derivatives. The functionalised CMC may be useful as a woundcare material.

### BACKGROUND

It is estimated that 1 to 2% of the population will experience a chronic wound during their lifetime in developed countries and this rate is increasing year after year. Stalled wounds may persist for months, even years before healing. Stalled wounds also have a psychological and social impact on patients: infection, delays in wound healing and wound-associated pain strongly affect patients' life.

Currently, common treatments of chronic wounds rely on wound dressings tailored to the state of the wound; often described as deep or shallow, clean or infected, and dry or exudative (Abrigo et al., 2014). Various wound dressings have been engineered to fit specific wounds based on the wound's characteristics. Of note, chronic wounds exhibit many symptoms which limit wound dressing treatment outcomes because they have been developed for treating a finite number of wound conditions concurrently. Although the advances in biomaterials and biomedical devices are making great strides, there is still a lack of understanding in the underlying molecular basis involved in failed tissue repair. Moreover, a lack of analytical assessments for wound diagnosis significantly impedes clinical success. Therefore, a biomedical device capable of monitoring wound parameters noninvasively at the wound site and integrated into a feedback control system (providing a closed loop therapeutic system; Ward et al., 2020) offers an alternative treatment approach for effective wound management to decrease healing time and mortality rates (Mehmood et al., 2014).

There is a clear need for improved outcome and cost-savings on woundcare materials, and new processes for manufacturing advanced wound care materials could cut the high cost of associated nursing and hospital resources. For these reasons, reducing healing time and pain for patients has become a priority.

### Click Reactions

Click reactions are inspired by nature's ability to form biosynthetic products through carbon-heteroatom bonds and is used to describe a series of coupling reactions, in which small modular units are joined together via heteroatom linkages (Kolb et al., 2001). For a reaction to be classified as a ‘click’ reaction, the reaction must be modular and involve the linkage of small modular units together. The reaction must also be high yielding, stereospecific, have a high thermodynamic driving force (>20 kJmol⁻¹) and produce inoffensive by-products which can be removed by non-chromatographic purification methods (Kolb et al., 2003).

Some click reactions are biorthogonal coupling reactions. A bioorthogonal coupling reaction is one that can occur outside or inside of living systems without interfering with native biochemical processes, and proceeds at a fast rate under physiological conditions. This two-step process allows for selective coupling and is applicable to a diverse range of biomolecules such as glycans, proteins and lipids.

Bioorthogonal coupling chemistry has been focused on the use of azides as bioconjugates due to their small size, and high stability in physiological conditions (Saxon et al., 2000a). Examples of such bioorthogonal coupling reactions that use an azide as the bioconjugate include the Huisgen strain-promoted 1,3-dipolar azide-alkyne cycloaddition (SPAAC) (Agard et al., 2004) and Staudinger ligation (Saxon et al., 2000b).

### Strain Promoted Azide-Alkyne [3+2] Cycloaddition (SPAAC)

The strain promoted azide-alkyne [3+2] cycloaddition (SPAAC) is a bioorthogonal approach to the copper (I)-catalysed Huisgen azide-alkyne 1,3-dipolar cycloaddition (CuAAC) to afford 1,4-substituted triazoles. SPAAC uses an alkyne functionality strained within a cyclooctyne, eliminating the requirement of a toxic Cu (I) catalyst (Agard et al., 2004). Cyclooctyne derivatives were identified as the most suitable alkyne reactant, given their high degree of intrinsic ring-strain (18 kJmol⁻¹) and their highly selective reactivity towards azides under physiological conditions (Agard et al., 2004).

A significant increase in the rate of azide-cyclooctyne coupling is achieved by installation of fluorine substituents adjacent to the alkyne (Codelli et al., 2008). In difluorinated cyclooctynes, the two electron withdrawing fluorine atoms contribute to an increased coupling rate by lowering the energy of the lowest unoccupied molecular orbital (LUMO) of the alkyne, which in turn increases the interaction energy with the highest occupied molecular orbital (HOMO) of the azide. In recent years, the use of difluorinated cyclooctynes (Codelli et al., 2008) and biarylazacyclooctynes (Jewett et al., 2010) has become increasingly standard for SPAAC.

### Staudinger Ligation

The Staudinger ligation is a reaction of an azide with a triarylphosphine, to form an iminophosphorane (an aza-ylide) at room temperature (Staudinger et al., 1919). The reaction was identified as a potentially useful coupling reaction, given the bioorthogonal nature of both the azide and phosphine reactants, along with the Staudinger reaction proceeding under mild conditions at extremely fast rates (Saxon et al., 2000a, 2000b). However, the mechanism of the Staudinger reaction proceeds through the formation of a covalent aza-ylide adduct, which is extremely susceptible to hydrolysis.

The Staudinger ligand 3-(diphenylphosphino)-4-(methoxycarbonyl)-phenyl was designed (Scheme 1) to intercept the iminophosphorane and prevent aza-ylide hydrolysis (Saxon et al., 2000a). An electrophilic trap, in the form of an ester, was installed ortho to the phosphane. The electrophilic trap functions by capturing the resulting nucleophilic aza-ylide by intramolecular cyclization to form an amide bond, which is both stable to hydrolysis and formed before competing aza-ylide hydrolysis can take place. Moreover, the reaction between the Staudinger ligand and an azide to produce a phospha-aza-ylide proceeds with extremely fast rates and high yields under physiological conditions.

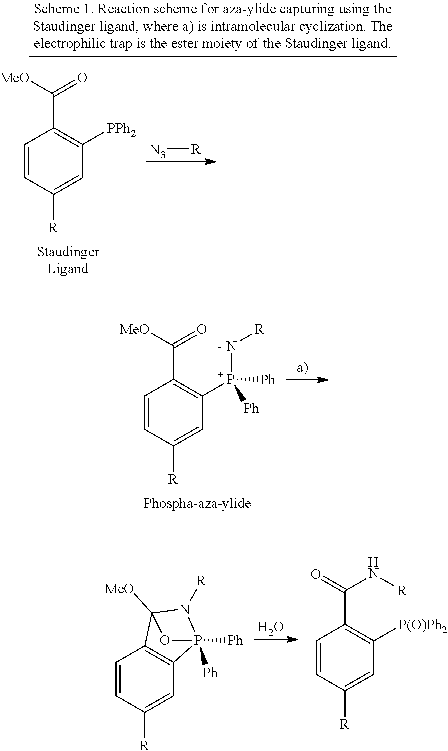

U.S. Pat. No. 8,512,728 B2 discloses a method for in situ formation of a medical device on biological tissue. The method includes attaching a plurality of reactive members of a primary binding pair on the surface of a biological tissue and reacting these members via a click reaction with a plurality of complementary reactive members of the primary binding pair on fibres. Simultaneously, the method includes a reactive members and complementary reactive members of an orthogonal secondary binding pair on the fibres that react via a click reaction to bind the fibres together.

U.S. Pat. No. 9,180,221 B2 discloses a method for adhering a medical device on biological tissue. The method includes providing a bifunctional medical adhesive with a plurality of reactive members of a first specific binding pair and a plurality of reaction members of a second specific binding pair. The biological tissue has a plurality of complementary reaction members to the first specific binding pair, whilst the medical device has a plurality of complementary reaction members to the second specific binding pair. The method includes contacting the bifunctional adhesive to the biological tissue to react the reactive members of the first specific binding pair via a click reaction, and then contacting the medical device to the bifunctional adhesive to reaction the reactive members of the second specific binding pair via another click reaction, thus binding the medical device to the biological tissue.

U.S. Pat. No. 9,510,810 B2 discloses a method for bonding a polymeric medical device on biological tissue with all claims directed to a kit for the method. The kit includes a polymeric medical device with a plurality of reactive members of a specific binding pair and a solution or suspension of a plurality of complementary reactive members of the specific binding pair with a linker that adheres to the biological tissue upon contact.

The present invention has been devised in the light of the above considerations.

### SUMMARY OF THE INVENTION

The invention includes the combination of the aspects and preferred features described except where such a combination is clearly impermissible or expressly avoided.

The inventors have developed chemical methods that allow the high-throughput functionalisation of cell culture scaffolds and wound dressings. The manufacture of these advanced functional materials will use the application of “one-pot” non-toxic yet highly specific chemistry to link bioactive molecules and cells to biomaterials. This methodology will be used to build a library of functionalised CMC materials, with a focus on hydrogels for healing problematic and chronic wounds.

In a first aspect, the invention may provide a method of functionalising sodium carboxymethylcellulose (Na-CMC), which is a cellulose comprising at least one unit of Formula 0:

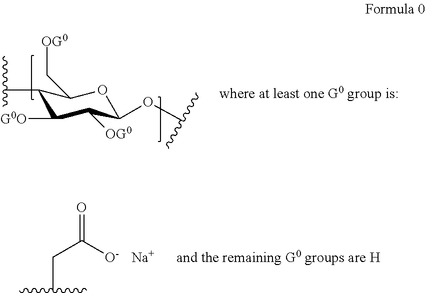

the method comprising the steps of:
i) coupling at least one said unit of Formula 0 with a spacer selected from:

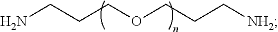

where n is an integer from 0 to 450; to form a CMC derivative comprising at least one unit of Formula 1:

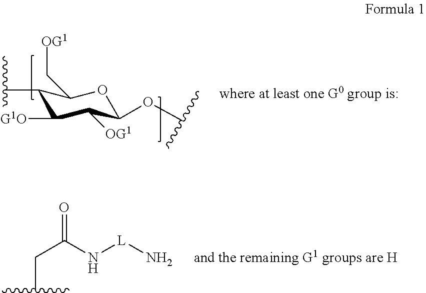

wherein L is:

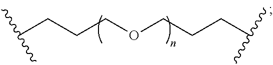

derived from the spacer;
ii) then coupling at least one unit of Formula 1 in said CMC derivative with a reactive member reagent selected from:

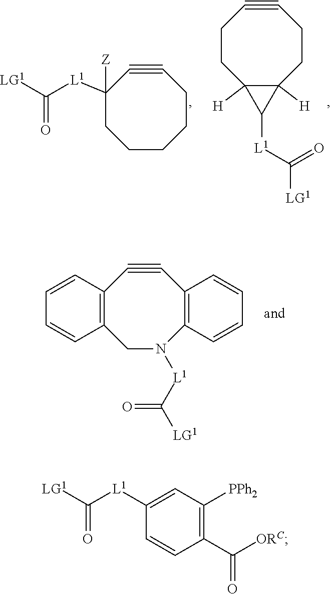

wherein:
LG¹ is a leaving group;
L¹ is a bond, alkylene, -(alkylene)O—*, —O(alkylene)-*, or -(alkylene)C(O)—*; wherein the*is the attachment to the cyclooctyne or benzene ring;
Z is —F, —Cl, —Br, or —I; and
R^{C} is alkyl;
   to form a CMC derivative comprising at least one unit of Formula 2:

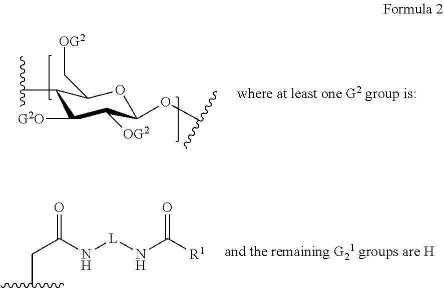

wherein:
R¹ is a reactive member selected from:

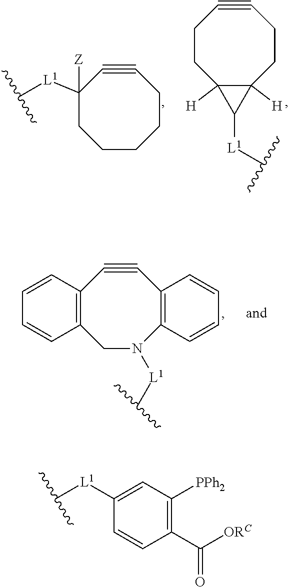

derived from the reactive member reagent;
iii) coupling at least one unit of Formula 2 in said CMC derivative with a compound of Formula B1:

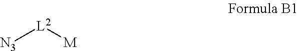

wherein:
L² is an alkylene, -(alkylene)C(O)—*, -(alkylene)NHC(O)—*, —(CH₂CH₂O)ₘCH₂—*, —CH₂(OCH₂CH₂)ₘ—*, arylene, or a bond, wherein the*is the attachment to M and m is an integer between 1 to 450; and
M is a biomolecular unit;
   wherein the azide functional group of the compound of Formula B1 reacts with R¹ of the at least one unit of Formula 2 in said CMC derivative via a click reaction to form a CMC derivative product comprising at least one unit of Formula 3:

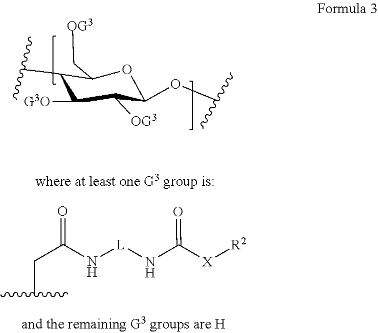

wherein:
X is a linker group selected from:

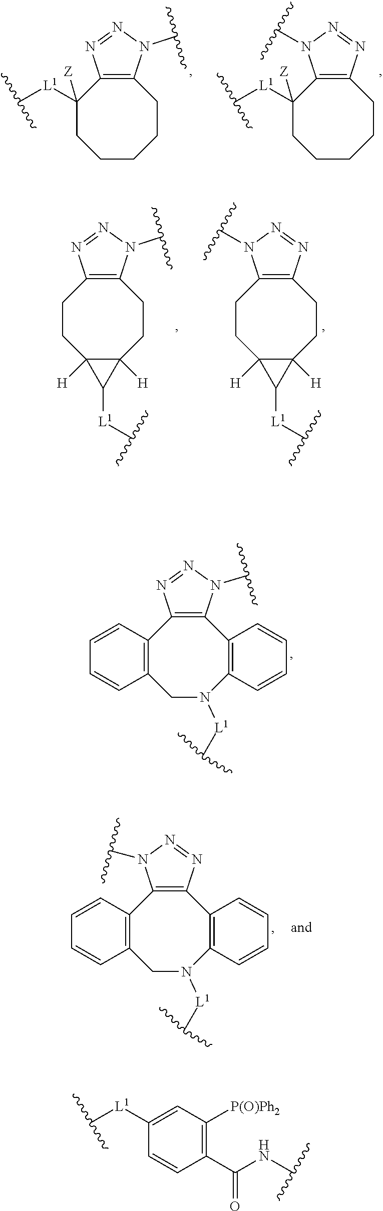

derived from R¹; and
R² is —L²—M derived from the compound of Formula B1.

In some embodiments, the method step i) comprises the following steps:
i) a) ion exchanging the Na⁺ in Na-CMC with a R₄N⁺ cation to form a CMC derivative comprising at least one unit of Formula 1ab:

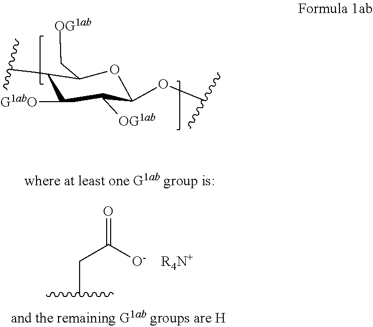

wherein R is alkyl; and
i) c) reacting at least one said unit of Formula 1ab with the spacer to form the CMC derivative comprising at least one unit of Formula 1, wherein the spacer and the CMC derivative comprising at least one unit of Formula 1 are as defined in the first aspect.

In some embodiments, the method step i) comprises the following steps:
i) aa) ion exchanging the Na⁺ in Na-CMC with a H⁺ to form a CMC derivative comprising at least one unit of Formula 1aa:

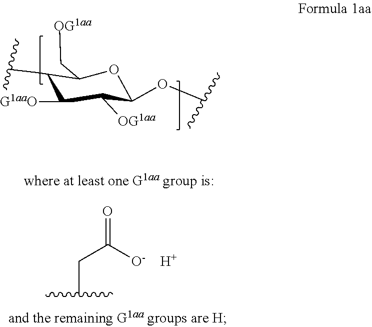

i) ab) ion exchanging the H⁺ of at least one said unit of Formula 1aa with a R₄N⁺ cation to form a CMC derivative comprising at least one unit of Formula 1ab:

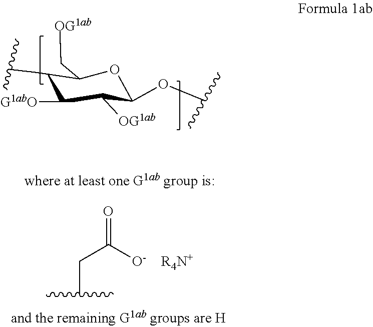

wherein R is alkyl; and
i) c) reacting at least one said unit of Formula 1ab with the spacer to form the CMC derivative comprising at least one unit of Formula 1, wherein the spacer and the CMC derivative comprising at least one unit of Formula 1 are as defined in the first aspect.

In some embodiments, the method step i) comprises the following steps:
i) b) activating at least one —CH₂COO⁻Na⁺ group of Na-CMC, at least one —CH₂COO⁻H⁺ group of the CMC derivative comprising at least one unit of Formula 1aa if present, or at least one —CH₂COO⁻R₄N⁺ group of the CMC derivative comprising at least one unit of Formula 1ab if present, by reacting with an activating reagent to form a CMC derivative comprising at least one unit of Formula 1b:

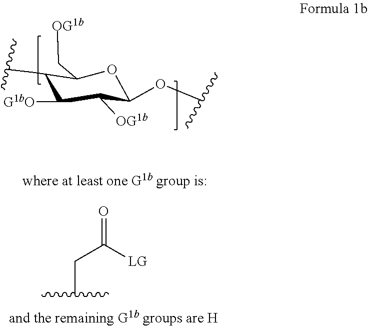

wherein LG is a leaving group; and
i) c) reacting the CMC derivative comprising at least one unit of Formula 1b with the spacer to form the CMC derivative comprising at least one unit of Formula 1, wherein the spacer and the CMC derivative comprising at least one unit of Formula 1 are as defined in the first aspect.

In some embodiments of the method step i) b) comprising Formula 1b, LG is selected from —F, —Cl, —Br, —I, —OR^{LG}, —SR^{LG}, —N⁺R^{LG}₃, —OC(O)R^{LG} or —OC(O) OR^{LG}; wherein R^{LG} is alkyl, aryl, mesyl, tosyl, triflyl, or

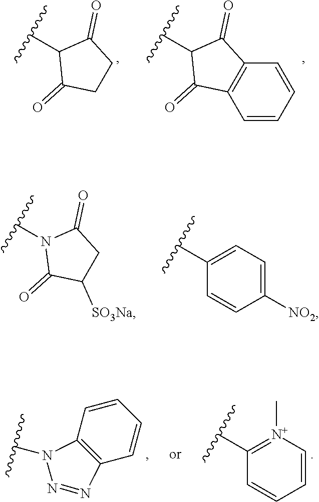

In some embodiments, the degree of substitution (DoS) of units of Formula 1 in the CMC derivative per D-anhydroglucopyranose monomers of cellulose after step i) is at least about 0.25.

In some embodiments, the reactive member reagent is selected from:

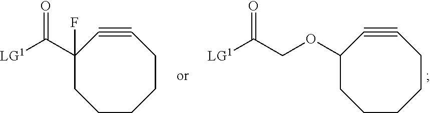

R¹ is selected from:

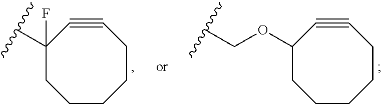 [SMILES: *C1(F)C#CCCCCC1 ; *COC1C#CCCCCC1]

and X is selected from:

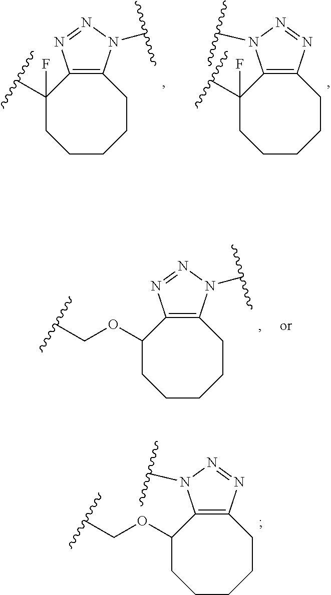 [SMILES: *n1nnc2c1CCCCCC2(*)F ; *n1nnc2c1C(*)(F)CCCCC2 ; *COC1CCCCCc2c1nnn2* ; *COC1CCCCCc2nnn(*)c21]

wherein:
LG¹ is —OH or

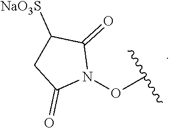

In some embodiments, the reactive member reagent is:

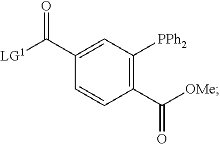

R¹ is:

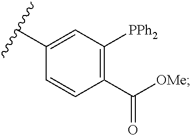

### and X is:

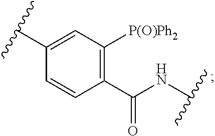

wherein:
LG¹ is —OH or

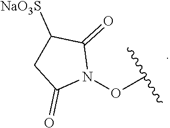

In some embodiments, the biomolecular unit M is a saccharide moiety, a metal chelator, a fluorescent probe, or a peptide.

In some embodiments wherein the biomolecular unit M is a saccharide moiety, the saccharide moiety is derived from a saccharide selected from N-acetylglucosamine (GlcNAc), N-acetylmannosamine (ManNAc) and peracetylated N-acetylmannosamine (Man(NAc)Ac₄).

In some embodiments wherein the biomolecular unit M is a metal chelator, the metal chelator is a Zn(II) chelator.

In some embodiments wherein the biomolecular unit M is a metal chelator, M is:

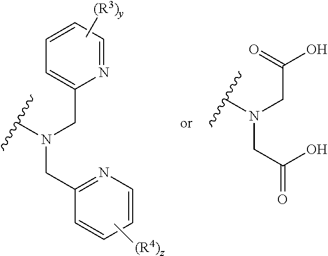

wherein:
y and z are each independently an integer from 0 to 4; and
each R³ and R⁴ is independently selected from —F, —Cl, —Br, and —I, —NO₂, —CO₂H, —CO₂(alkyl), —CN, alkyl, aryl, and acyl; or, if one or both of y and z is an integer from 2 to 4, two R³ groups or two R⁴ groups on adjacent carbon atoms are linked to form a fused benzene ring.

In some embodiments wherein the biomolecular unit M is a fluorescent probe, M is:

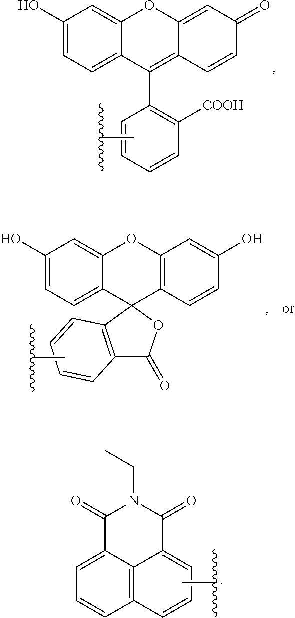

In some embodiments wherein the biomolecular unit M is a peptide, M is:

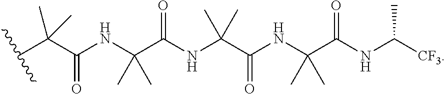 [SMILES: *C(C)(C)C(=O)NC(C)(C)C(=O)NC(C)(C)C(=O)NC(C)(C)C(=O)N[C@H](C)C(F)(F)F]

In a further aspect, the invention may provide a CMC derivative product obtainable by the method of the first aspect, wherein the CMC derivative product comprises at least one unit of Formula 3:

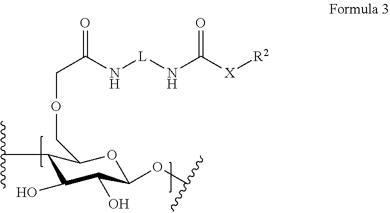

wherein:
L is:

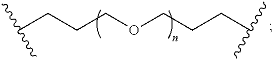

n is an integer from 0 to 450;
X is selected from:

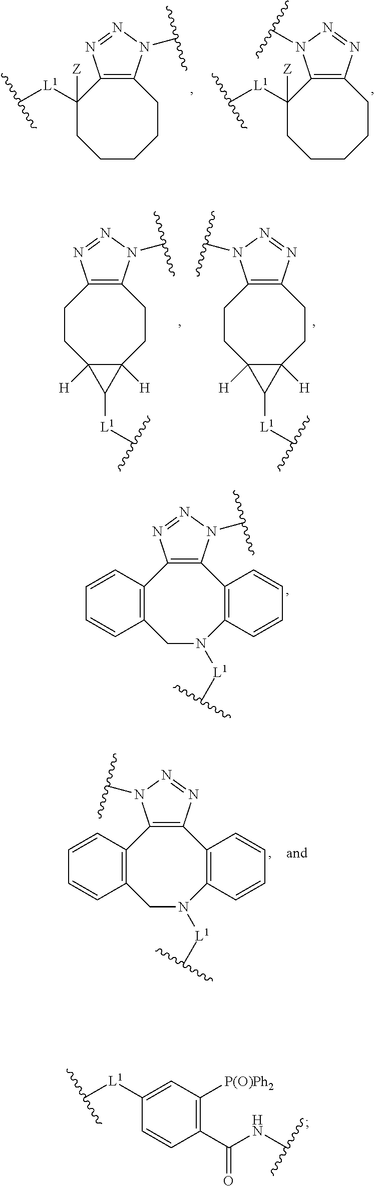

L¹ is a bond, alkylene, -(alkylene)O—*, —O(alkylene)-*, or -(alkylene)C(O)—*; wherein the*is the attachment to the cyclooctene or benzene ring;
Z is —F, —Cl, —Br, or —I;
R² is —L²—M;
L² is an alkylene, -(alkylene)C(O)—*, -(alkylene)NHC(O)—*, —(CH₂CH₂O)ₘCH₂—*, —CH₂(OCH₂CH₂)ₘ—*, arylene, or a bond, wherein the*is the attachment to M and m is an integer between 1 to 450; and
M is a biomolecular unit.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The inventors have identified the need for simple chemistry/biochemistry technology that will add value to healthcare products, and have developed high yielding and cost-efficient methods for adding reactive groups to wound healing materials. Another area of need is the addition of biosensing groups that can identify a disease state. The methodology will permit the functionalisation of wound healing materials with any moiety that has an azide functional group without the need to add a metal ion catalyst (which can have unwanted biological effects, like toxicity).

The invention may enable the creation of a tool kit by which any molecule or biomolecule of medical interest in relation to wound healing can be coupled to the surface of these activated CMC materials. These coupling reactions can be performed under “one-pot” and non-toxic conditions.

### CMC

Cellulose is a polysaccharide homopolymer comprising D-anhydroglucopyranose monomers derived from D-glucopyranose joined by an ether group at the C-1 and C-4 positions (known as β-(1,4)-glycosidic bonds; O'Sullivan et al., 1997) of each of the D-anhydroglucopyranose monomers.

Carboxymethylcellulose (CMC) is a cellulose derivative with carboxymethyl groups (—CH₂COOH) bound to some (up to all) of the C-2, C-3 and C-6 hydroxyl groups of the D-anhydroglucopyranose monomers that make up the cellulose backbone (Pettignano et al., 2019). Typically, the carboxymethyl groups are bound to the C-2 or C-6 hydroxyl groups. Lower amounts of the carboxymethyl groups are bound to the C-3 hydroxyl groups due to its lower reactivity (Pettignano et al., 2019). When referring to CMC herein, the D-anhydroglucopyranose monomers of the cellulose backbone are randomly substituted with units of CMC, from 1 to n units where n is the total number of D-anhydroglucopyranose monomers, unless stated otherwise. This is quantified as the degree of substitution (DoS) of units of CMC per D-anhydroglucopyranose monomers of cellulose.

[For example, a DoS of 0.5 means that 50% of the monomer units in the polymeric chain are D-anhydroglucopyranose monomers and the other 50% have the carboxymethyl substitution. A DoS of 1 means that on average every monomer has a carboxymethyl substitution.]

Typically, the DoS of units of CMC per D-anhydroglucopyranose monomers of cellulose can be quantified as a range from about 0.4 to about 1.5, such as from about 0.65 to about 1.45, such as from about 0.7 to about 1.2.

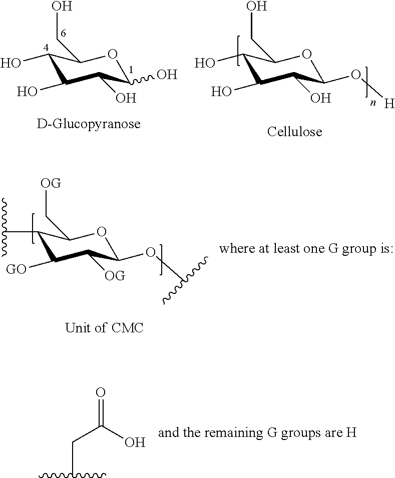

The polymer chain length of CMC is defined in terms of average molecular weight (MW). Typically, the average MW of CMC may be from about 90,000 Da to about 700,000 Da. In some embodiments, the average MW of CMC may be about 250,000 Da.

CMC may also be either soluble or insoluble (for example, fibrous, nanoparticulate, or colloidal). The CMC functionalised in the methods of the present invention may be applied to any of the forms of CMC.

CMC is typically a hydrogel. Hydrogels are a network of crosslinked polymer chains that are hydrophilic. CMC-based hydrogels are functionalised using the methodology described herein to couple a biomolecule via a spacer.

### Na-CMC

CMC is often used in the form of its sodium salt, sodium carboxymethylcellulose (Na-CMC), which can be water-soluble or form a gel in water. The D-anhydroglucopyranose monomers of the cellulose backbone are randomly substituted with units of Na-CMC (Formula 0), from 1 to n units where n is the total number of D-anhydroglucopyranose monomers, unless stated otherwise. This is quantified by the degree of substitution (DoS), of units of Na-CMC per D-anhydroglucopyranose monomer of the cellulose. Typically, the DoS of units of Na-CMC per D-anhydroglucopyranose monomers of cellulose can be quantified as a range from about 0.4 to about 1.5, such as from about 0.65 to about 1.45, such as from about 0.7 to about 1.2. In some embodiments, the DoS of units of Na-CMC may be from about 1.15 to about 1.45. In some embodiments, the DoS of units of Na-CMC may be from about 0.65 to about 0.9. Typically, the DoS of units of Na-CMC is about 0.7.

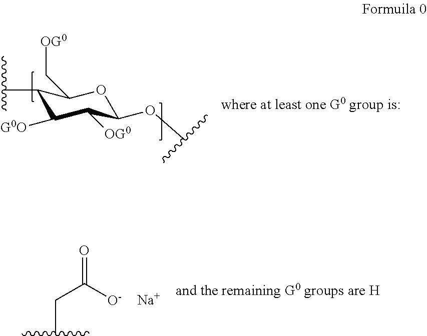

The loading of sodium cations (Na⁺) in Na-CMC may be from about 4% to about 15%, such as from about 6.5% to 14.5%, such as from about 7% to about 12%. In some embodiments, the loading of Na⁺ may be from about 10.4% to about 12%. In some embodiments, the loading of Na⁺ may be from about 6.5% to about 9.5%.

When referring to Na-CMC herein, Na-CMC is a cellulose comprising at least one unit of Formula 0.

The carboxymethyl functionality at the C-6 position of D-anhydroglucopyranose increases the hydrophilicity of Na-CMC by weakening the strength of intramolecular hydrogen bond interactions. This in turn increases the solubility of Na-CMC in aqueous conditions so Na-CMC can absorb wound exudate and form a cohesive gel. This gelled state adheres the wound dressing, in the form of woven Na-CMC fibres, to the surface of the wound and promotes accelerated wound healing by reducing dead space where bacteria can grow; maintaining moisture balance at the site of the wound; and locking in exudate and trapping bacteria (Queen et al., 2011).

### Functionalisation of CMC

The present application demonstrates the coupling of a spacer to a CMC backbone and subsequent coupling with a biomolecule to form new classes of functionalised CMC hydrogels. Typically, this starts with functionalising Na-CMC.

In the first step of this methodology, the CMC is coupled to a spacer to form a CMC-spacer compound. The CMC-spacer compound is a CMC derivative comprising at least one unit of Formula 1:

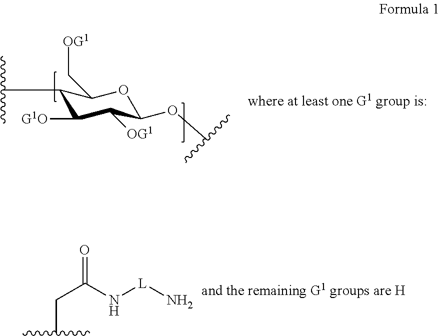

wherein L is derived from the spacer. The typical DoS of units of Formula 1 in the CMC derivative per D-anhydroglucopyranose monomers of cellulose for the first step of this methodology is from about 0.26 to about 0.59. This corresponds to a typical degree of conversion (DoC) per available carboxymethyl carboxylates of CMC of from about 0.37 to about 0.84.

In the second step of this methodology, the CMC-spacer compound is coupled to a reactive member reagent to form a CMC-reactive member compound. The CMC-reactive member compound is a CMC derivative comprising at least one unit of Formula 2:

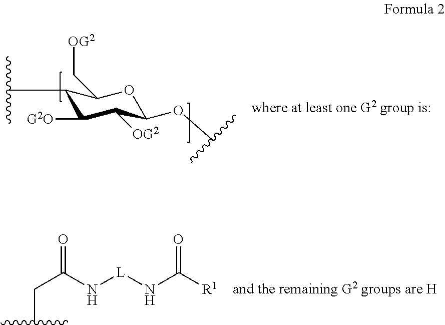

wherein L is derived from the spacer, and R¹ is a reactive member. The typical DoS of units of Formula 2 in the CMC derivative per D-anhydroglucopyranose monomers of cellulose for the second step of this methodology is from about 0.17 to about 0.18. This corresponds to a typical DoC per available amines of a CMC derivative comprising at least one unit of Formula 1 of from about 0.66 to about 0.71.

In a third step of this methodology, the CMC-reactive member compound is coupled to a biomolecule comprising a complementary reactive member (generally, an azide, —N₃) via a click reaction to form a CMC-biomolecule compound. The CMC-biomolecule compound is a CMC derivative product comprising at least one unit of Formula 3:

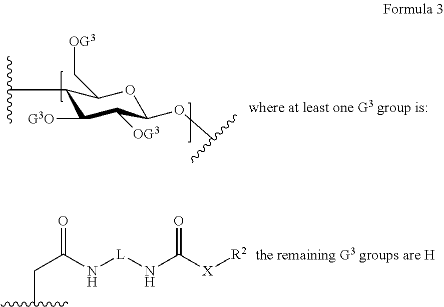

wherein L is derived from the spacer, X is derived from the click reaction between the reactive member R1 and the complementary reactive member, and R² is derived from the biomolecule. The typical DoS of units of Formula 3 in the CMC derivative per D-anhydroglucopyranose monomers of cellulose for the third step of this methodology is from about 0.04 to about 0.06. This corresponds to a typical DoC per available reactive member R¹ of a CMC derivative comprising at least one unit of Formula 2 of from about 0.26 to about 0.33.

The conversions of the successive reactions in the first, second and third steps are unlikely to be 100%. The conversion between Na-CMC to a CMC derivative comprising at least one unit of Formula 1 may be from about 37% to about 84% conversion. The conversion between a CMC derivative comprising at least one unit of Formula 1 and a CMC derivative comprising at least one unit of Formula 2 may be from about 66% to about 71% conversion. The conversion between a CMC derivative comprising at least one unit of Formula 2 and a CMC derivative comprising at least one unit of Formula 3 may be from about 26% to about 33% conversion.

The methodology described herein may enable the functionalisation of CMC to produce materials for a wound monitoring device. A wound monitoring device would help decrease prolonged hospitalization, multiple doctors' visits, and the expensive lab testing associated with the diagnosis and treatment of chronic wounds. A device capable of monitoring the wound status and stimulating the healing process is highly desirable.

The methodology described herein may also add value to CMC materials by covalently linking bioactive molecules and cells to wound healing dressings composed of CMC. The inventors envisage the methodology described herein may have application in reactive oxygen species (ROS) sensing as an indicator of inflammation and/or healing, complex saccharides as sites for immune cell recruitment, cell immobilisation to create a skin-like coating and metal chelation to reduce the activity of protease enzymes such as matrix metalloproteinases.

The inventors envisage creating a tool kit by which a CMC hydrogel wound dressing can be coupled to any biomolecule, molecular probe or living cell that possesses an azide functional group, by use of bioorthogonal coupling chemistry.

### Step i) Coupling of Na-CMC to a Spacer

A spacer can be used to introduce a fixed distance between a solid support, such as a CMC hydrogel like Na-CMC, and a reactive terminus. This is particularly useful when the direct connection of a molecule to a solid support would lead to becoming sterically hindered by the solid support, or to introduce a new type of reactive terminus to the solid support for subsequent coupling reactions.

An example of a commonly used spacer group in biomaterials is polyethylene glycol (PEG), which is a polymer of repeating ethylene glycol monomers that is commercially available in a range of chain lengths, and reactant terminus. PEG has become commonly used in the production of medical biomaterials due to its non-toxic, non-immunogenic and hydrophilic properties (Soyez et al., 1996). Furthermore, PEG is also highly flexible in structure and can provide flexibility in highly rigid solid supports.

The first step in the method of functionalising Na-CMC is coupling Na-CMC with a spacer selected from:

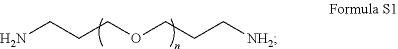

wherein n is an integer from 0 to 450 to form a CMC derivative comprising at least one unit of Formula 1:

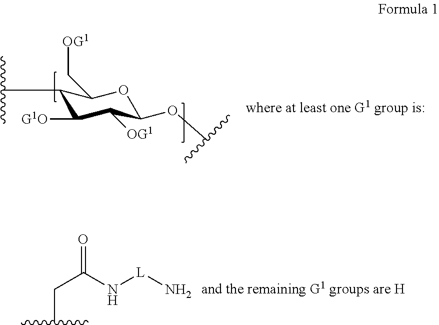

wherein L is:

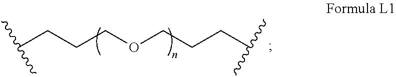

derived from the spacer. This step will be referred to as step i).

A CMC derivative comprising at least one unit of Formula 1 may be referred to as a CMC-spacer compound.

In some embodiments, there is one at least one G¹ group in Formula 1. In some embodiments, the at least one G¹ group is on the C-6 hydroxyl group of Formula 1, such that the remaining C-2 and C-3 G¹ groups are H. In some embodiments, the at least one G¹ group is on the C-2 hydroxyl group of Formula 1, such that the remaining C-3 and C-6 G¹ groups are H. In some embodiments, the at least one G¹ group is on the C-3 hydroxyl group of Formula 1, such that the remaining C-2 and C-6 G¹ groups are H.

The group L is derived by the spacer. In some embodiments, the spacer is Formula S1 and L is Formula L1.

In some embodiments, n is an integer from 0 to 450. In some embodiments, n is an integer from 1 to 10. In some embodiments, n is an integer from 1 to 5. In some embodiments, n is an integer from 1 to 3. In some embodiments, n is an integer from 2 to 3. In some embodiments, n is 3. In some embodiments, n is 2. In some embodiments, n is 1. When referring to n, the range includes the lower and upper limits.

The chain length n may dictate the structural properties of the CMC derivative material. In some embodiments when n is 2, the material made of the CMC derivative comprising at least one unit of Formula 1 may be brittle. In some embodiments when n is 3, the material made of the CMC derivative comprising at least one unit of Formula 1 may be compressible.

In step i), a base may be added to capture any acid generated. The base used may be dry or anhydrous to prevent side reactions with water present. In some embodiments, the base is a tertiary amine. In some embodiments, the base is a triethylamine (Et₃N).

In step i), the reaction solvent may be a polar (hydrophilic) aprotic solvent. The reaction solvent may be N,N′-dimethylformamide (DMF), hexamethylphosphoramide (HMPA), dimethylsulfoxide (DMSO), dihydrolevoglucosenone (Cyrene™), tetrahydrofuran (THF), or acetonitrile (MeCN). In some embodiments, the reaction solvent is N,N′-dimethylformamide (DMF). The reaction solvent may be dry or anhydrous.

When the reaction in step i) is complete, the CMC derivative comprising at least one unit of Formula 1 may be precipitated out of solution. The solvent to induce precipitation may be acetone, particularly when the reaction solvent used for the reaction was N, N′-dimethylformamide (DMF). In some embodiments, the CMC derivative comprising at least one unit of Formula 1 may be placed under reduced pressure, such as a vacuum, to remove any residual solvent. In some embodiments, the CMC derivative comprising at least one unit of Formula 1 may be freeze-dried (lyophilized) to remove any residual solvent.

The degree of substitution (DoS) of units of Formula 1 in the CMC derivative per D-anhydroglucopyranose monomers of cellulose may be quantified using elemental analysis, a ninhydrin assay (Kaiser Test) or a 2,4,6-trinitrobenzenesulfonic acid (TNBS) assay. In some embodiments, the DoS of units of Formula 1 in the CMC derivative may be a range from about 0.26 to about 0.59, such as from about 0.35 to about 0.58. Typically, the DoS of units of Formula 1 in the CMC derivative is about 0.35 to about 0.58.

Preferably, the DoS of units of Formula 1 in the CMC derivative is at least about 0.1, such as at least about 0.15, such as at least about 0.2, such as at least about 0.25, such as at least about 0.3.

A DoS of units of Formula 1 in the CMC derivative per D-anhydroglucopyranose monomers of cellulose for step i) of from about 0.26 to about 0.59 corresponds to a DoC per available carboxymethyl carboxylates of CMC of from about 0.37 to about 0.84.

The conversion between Na-CMC to a CMC derivative comprising at least one unit of Formula 1 may be from about 37% to about 84% conversion.

In some embodiments, this step i) may comprise the sub-steps of ion exchange steps i) a), i) aa), or i) ab); activation step i) b); or reaction with the spacer step i) c).

### Ion Exchange Step(s)—Steps i) a), i) aa) or i) ab)

Na-CMC shows limited solubility in organic solvents (e.g. DMF or DMSO) which may limit chemical synthesis (Ernsting et al., 2011). In some embodiments, the first step in the method of coupling Na-CMC with a spacer comprises an ion exchange. An ion exchange is a process of exchanging one ion (anion or cation) with another ion of the corresponding charge (anion or cation). The general purpose of the ion exchange step, or steps, is to solubilise the CMC derivative in an organic solvent.

In some embodiments, the ion exchange step is the process of exchanging a cation with another cation. The ion exchange step may comprise an ion exchange of the sodium cation (Na⁺) in Na-CMC with a R₄N⁺ cation, wherein R is alkyl, such as methyl, ethyl, n-propyl, or n-butyl to form a CMC derivative comprising at least one unit of Formula 1ab (step i) a) in Scheme 1). The R₄N⁺ cation may be a tetraalkylammonium cation such as tetrabutylammonium cation ((nBu)₄N⁺ or TBA).

The ion exchange step may comprise an ion exchange of the sodium cation (Na⁺) in Na-CMC with a proton (H⁺), to form a CMC derivative comprising at least one unit of Formula 1aa (step i) aa) in Scheme 1). The ion exchange step may comprise an ion exchange of the proton (H⁺) with a R₄N⁺ cation, wherein R is alkyl, such as methyl, ethyl, n-propyl, or n-butyl, to form a CMC derivative comprising at least one unit of Formula 1ab (step i) ab) in Scheme 1). The R₄N⁺ cation may be a tetraalkylammonium cation such as tetrabutylammonium cation ((nBu)₄N⁺ or TBA). The ion exchange step may comprise both step i) aa) and step i) ab) sequentially. The ion exchange step may comprise step i) a) only.

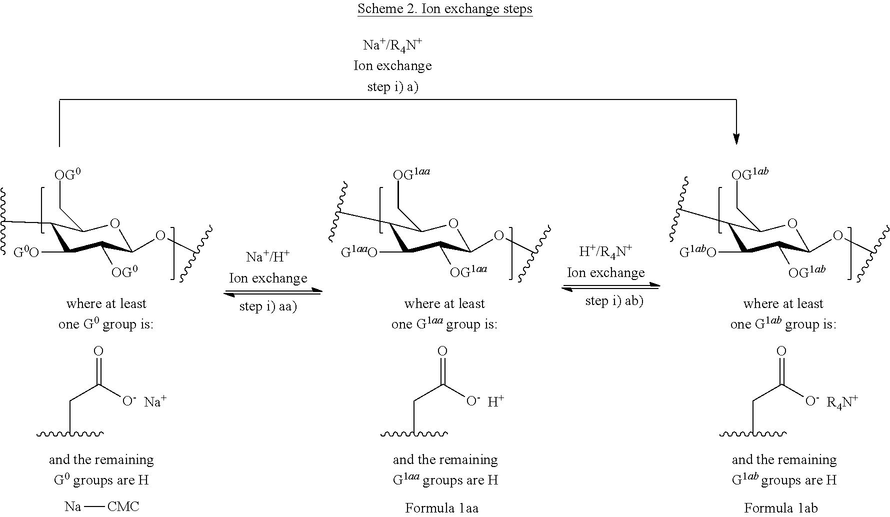

In some embodiments, there is one at least one G^{1aa} group in Formula 1aa. In some embodiments, the at least one G^{1aa} group is on the C-6 hydroxyl group of Formula 1aa, such that the remaining C-2 and C-3 G^{1aa} groups are H. In some embodiments, the at least one G^{1aa} group is on the C-2 hydroxyl group of Formula 1aa, such that the remaining C-3 and C-6 G^{1aa} groups are H. In some embodiments, the at least one G^{1aa} group is on the C-3 hydroxyl group of Formula 1aa, such that the remaining C-2 and C-6 G^{1aa} groups are H.

In some embodiments, there is one at least one G^{1ab} group in Formula 1ab. In some embodiments, the at least one Gab group is on the C-6 hydroxyl group of Formula 1ab, such that the remaining C-2 and C-3 G^{1ab} groups are H. In some embodiments, the at least one G^{1ab} group is on the C-2 hydroxyl group of Formula 1ab, such that the remaining C-3 and C-6 G^{1ab} groups are H. In some embodiments, the at least one G^{1ab} group is on the C-3 hydroxyl group of Formula 1ab, such that the remaining C-2 and C-6 G^{1ab} groups are H.

The ion exchange steps i) a), i) aa) or i) ab) may be conducted using an ion exchange resin. The ion exchange resin may be a cation exchange resin. The ion exchange resin may be a strong acid cation exchange resin, such as DOWEX Monospheres 650 C, Amberlite 120, Amberlyst 15, and Amberlite IRC-50. In some embodiments, the strong acid cation exchange resin is DOWEX Monospheres 650 C.

In the ion exchange steps i) a), i) aa) or i) ab), the reaction solvent may be water, such as distilled water, such as Milli-Q water.

When the ion exchange in step i) a) or step i) ab) is complete, the solution comprising the CMC derivative comprising at least one unit of Formula 1ab may be at an alkaline pH. In some embodiments, the pH after the completion of step i) a) or step i) ab) is pH 7 or above. In some embodiments, the pH after step i) a) or step i) ab) is pH 8 or above. In some embodiments, the pH after step i) a) or step i) ab) is from about pH 8 to about pH 9.

When the ion exchange in steps i) a), i) aa) or i) ab) is complete, the CMC derivative comprising at least one unit of Formula 1a or Formula 1ab may be freeze-dried (lyophilized) to remove any residual solvent. The CMC derivative comprising at least one unit of Formula 1a or Formula 1ab may be freeze-dried for at least 24 h, at least 36 h, or at least 48 h. In some embodiments, the CMC derivative comprising at least one unit of Formula 1a or Formula 1ab may be freeze-dried for at least 48 h. In some embodiments, the CMC derivative comprising at least one unit of Formula 1ab may be freeze-dried for at least 48 h.

### Activation of Carboxymethyl Group and its Variants—Step i) b)

Coupling of the variants of the carboxymethyl group of a derivative CMC, such as Na-CMC or CMC derivatives comprising at least one unit of Formula 1aa or Formula 1ab, with the spacer can be facilitated with activation of the variant of the carboxymethyl group.

The variant of the carboxymethyl group in Na-CMC (Formula 0) is —CH₂COO⁻Na⁺. The variant of the carboxymethyl group in Formula 1aa is —CH₂COO⁻H⁺. The variant of the carboxymethyl group in Formula 1ab is —CH₂COO⁻RAN⁺. The variant of the carboxymethyl group in Na-CMC (Formula 0) or in Formula 1aa or Formula 1ab may be the methylenecarboxylate anion (—CH₂COO⁻) when ignoring the presence of the counterion.

The step of activating the variant of the carboxymethyl group of a derivative CMC, such as Na-CMC or CMC derivatives comprising at least one unit of Formula 1aa or Formula 1ab, may be by reacting with an activating reagent to form a CMC derivative comprising at least one unit of Formula 1b:

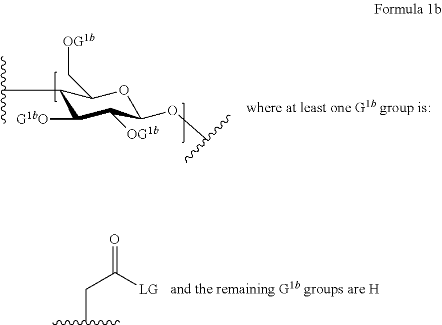

wherein LG is a leaving group. This activation step will be referred to as step i) b).

In some embodiments, there is one at least one G^{1b} group in Formula 1b. In some embodiments, the at least one G^{1b} group is on the C-6 hydroxyl group of Formula 1b, such that the remaining C-2 and C-3 G^{1b} groups are H. In some embodiments, the at least one G^{1b} group is on the C-2 hydroxyl group of Formula 1b, such that the remaining C-3 and C-6 G^{1b} groups are H. In some embodiments, the at least one G^{1b} group is on the C-3 hydroxyl group of Formula 1b, such that the remaining C-2 and C-6 G^{1b} groups are H.

A leaving group is a molecular fragment that departs with the pair of electrons in a heterolytic bond cleavage. The bond cleaved heterolytically when reacted with a spacer is the C-LG bond in Formula 1b. The skilled person will understand that a “better” leaving group is a leaving group that speeds up the reaction by having a low activation barrier to reacting with a nucleophile, such as the spacer. Preferably, the leaving group LG will be a “better” leaving group than the corresponding group of in the variant of the carboxymethyl group of Na-CMC (—O⁻Na⁺) or the CMC derivatives comprising at least one unit of Formula 1aa (—O⁻H⁺) or Formula 1ab (—O⁻R₄N⁺).

The skilled person will appreciate that the leaving group LG in Formula 1b is not particularly limited. The leaving group LG may be a halogen, —OR^{LG}, —SR^{LG}, —N⁺R^{LG}₃, —OC(O)R^{LG} or —OC(O)OR^{LG}; wherein R^{LG} is alkyl, aryl, methanesulfonyl (mesyl), toluenesulfonyl (tosyl), trifluoromethanesulfonyl (triflyl), or

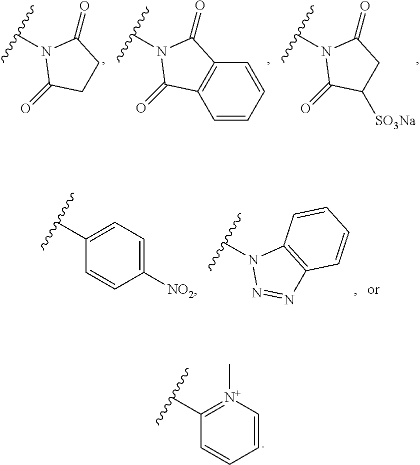

A halogen may be —F, —Cl, —Br, or —I. The leaving group LG may suitably be:

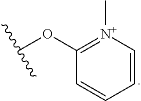 [SMILES: *Oc1cccc[n+]1C]

Alternatively, the leaving group LG may suitably be:

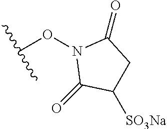

because this leaving group may allow the CMC derivative to swell better in solution, which may increase the reactivity with the spacer.

The skilled person will appreciate that the activating reagent is also not particularly limited. The activating reagent may be selected from thionyl chloride, oxalyl chloride, mesyl chloride, tosyl chloride, acetic anhydride, trifluoromethanesulfonic anhydride, N-hydroxysuccinimide, N-hydroxyphthalimide, N-hydroxysulfosuccinimide hydroxysulfosuccinimide sodium salt, 4-nitrophenol, HOBt, HBTU, HATU, PyBOP, a carbodiimide (such as EDCI, DCC or DIC), or 2-chloro-1-methylpyridinium iodide (CMP-I). In some embodiments, the activating reagent may be a combination thereof, such as N-hydroxysuccinimide with a carbodiimide, or N-hydroxyphthalimide with a carbodiimide, or N-hydroxysulfosuccinimide sodium salt with a carbodiimide, or 4-nitrophenol with a carbodiimide, or HOBt with a carbodiimide. The activating reagent may be 2-chloro-1-methylpyridinium iodide (CMP-I).

HOBt is hydroxybenzotriazole. HBTU is hexafluorophosphate benzotriazole tetramethyl uronium or O-(benzotriazol-1-yl)-N,N,N′,N′-tetramethyluronium hexafluorophosphate or 2-(1H-benzotriazol-1-yl)-1,1,3,3-tetramethyluronium hexafluorophosphate. HATU is hexafluorophosphate azabenzotriazole tetramethyl uronium or 1-[bis(dimethylamino)methylene]-1H-1,2,3-triazolo[4,5-b]pyridinium 3-oxide hexafluorophosphate. PyBOP is benzotriazol-1-yloxytripyrrolidinophosphonium hexafluorophosphate. EDCI is 1-ethyl-3-(3-dimethylaminopropyl)carbodiimide hydrochloride. DCC is N,N′-dicyclohexylcarbodiimide. DIC is N,N′-diisopropylcarbodiimide.

The amount of activating reagent added to the reaction may be sufficient to activate 78% of the carboxylate groups so the CMC derivative would maintain its gelling properties. In some embodiments, the amount of activating reagent added to the reaction may be at least a stoichiometric amount as compared to the initial amount of moles of Na-CMC used multiplied by the degree of substitution (DoS) of the Na-CMC.

In activation step i) b), the reaction may be cooled below room temperature or may be conducted at room temperature. In some embodiments, the reaction temperature is room temperature. In some embodiments, the reaction temperature is cooled to about −100° C. to about 10° C. In some embodiments, the reaction temperature is cooled to about −80° C. to about 10° C. In some embodiments, the reaction temperature is cooled to about −60° C. to about 10° C. In some embodiments, the reaction temperature is cooled to about −40° C. to about 10° C. In some embodiments, the reaction temperature is cooled to about −20° C. to about 10° C. In some embodiments, the reaction temperature is cooled to about 0° C. to about 10° C. In some embodiments, the reaction temperature is cooled to about 4° C. Preferably, the reaction temperature is cooled below about 4° C. to prevent cross-linking and the formation of interchain ester bonds in Na-CMC (Barbucci et al., 2005).

In activation step i) b), the reaction solvent may be a polar (hydrophilic) aprotic solvent. The reaction solvent may be N,N′-dimethylformamide (DMF), hexamethylphosphoramide (HMPA), dimethylsulfoxide (DMSO), dihydrolevoglucosenone (Cyrene™), tetrahydrofuran (THF), or acetonitrile (MeCN). In some embodiments, the reaction solvent is N,N′-dimethylformamide (DMF). The reaction solvent may be dry or anhydrous to prevent water from reacting with the activated CMC derivative comprising at least one unit of Formula 1b.

Reaction with the Spacer—Step i) c)

The derivatives of CMC comprising at least one unit of Formula 1aa, Formula 1ab or Formula 1b can be coupled with a spacer selected from:

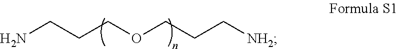

wherein n is an integer from 0 to 450; to form a CMC derivative comprising at least one unit of Formula 1:

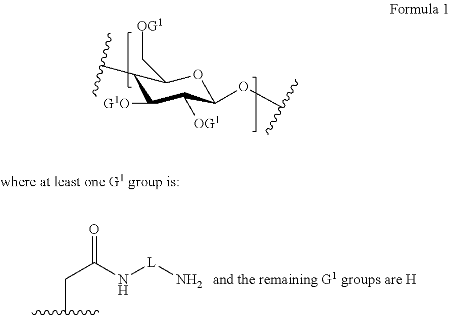

wherein L is:

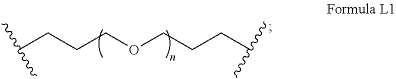

derived from the spacer, where n is an integer from 0 to 450, as described for Na-CMC in step i), by reaction with the spacer. This reaction step will be referred to as step i) c).

In some embodiments, the spacer is Formula S1 and L is Formula L1.

In some embodiments, n is an integer from 0 to 450. In some embodiments, n is an integer from 1 to 10.

In some embodiments, n is an integer from 1 to 5. In some embodiments, n is an integer from 1 to 3. In some embodiments, n is an integer from 2 to 3. In some embodiments, n is 3. In some embodiments, n is 2. In some embodiments, n is 1. When referring to n, the range includes the lower and upper limits.

The chain length n may dictate the structural properties of the CMC derivative material. In some embodiments when n is 2, the material made of the CMC derivative comprising at least one unit of Formula 1 may be brittle. In some embodiments when n is 3, the material made of the CMC derivative comprising at least one unit of Formula 1 may be compressible.

When reacting the spacer with a derivative of CMC comprising at least one unit of Formula 1aa, Formula 1ab or Formula 1b, a base may be added to capture any acid generated. The acid generated may be hydrogen iodide. The base used may be dry or anhydrous to prevent side reactions with water present. In some embodiments, the base is a tertiary amine. In some embodiments, the base is triethylamine (Et₃N).

In step i) c), the reaction solvent may be a polar (hydrophilic) aprotic solvent. The reaction solvent may be N,N-dimethylformamide (DMF), hexamethylphosphoramide (HMPA), dimethylsulfoxide (DMSO), dihydrolevoglucosenone (Cyrene™M), tetrahydrofuran (THF), or acetonitrile (MeCN). In some embodiments, the reaction solvent is N,N′-dimethylformamide (DMF). In some embodiments, the reaction solvent is the same as the reaction solvent used in step i) b). The reaction solvent may be dry or anhydrous to prevent water from reacting with the activated CMC derivative comprising at least one unit of Formula 1b if present.

When the reaction in step i) c) is complete, the CMC derivative comprising at least one unit of Formula 1 may be precipitated out of solution. The solvent to induce precipitation may be acetone, particularly when the reaction solvent used for the reaction was N, N′-dimethylformamide (DMF). In some embodiments, the CMC derivative comprising at least one unit of Formula 1 may be placed under reduced pressure, such as a vacuum, to remove any residual solvent. In some embodiments, the CMC derivative comprising at least one unit of Formula 1 may be freeze-dried (lyophilized) to remove any residual solvent.

The degree of substitution (DoS) of units of Formula 1 in the CMC derivative per D-anhydroglucopyranose monomers of cellulose may be quantified using elemental analysis, a ninhydrin assay (Kaiser Test) or a TNBS assay. In some embodiments, the DoS of units of Formula 1 in the CMC derivative may be a range from about 0.26 to about 0.59, such as from about 0.35 to about 0.58. Typically, the DoS of units of Formula 1 in the CMC derivative is about 0.35 to about 0.58.

Preferably, the DoS of units of Formula 1 in the CMC derivative is at least about 0.1, such as at least about 0.15, such as at least about 0.2, such as at least about 0.25, such as at least about 0.3.

A DoS of units of Formula 1 in the CMC derivative per D-anhydroglucopyranose monomers of cellulose for step i) of from about 0.26 to about 0.59 corresponds to a DoC per available carboxymethyl carboxylates of CMC of from about 0.37 to about 0.84.

The conversion between Na-CMC to a CMC derivative comprising at least one unit of Formula 1 may be from about 37% to about 84% conversion.

The first step in the method of functionalising Na-CMC, step i), may comprise the ion exchange step i) a) and the reaction with spacer step i) c). The first step in the method of functionalising Na-CMC, step i), may comprise the ion exchange step i) aa) and the reaction with spacer step i) c). The first step in the method of functionalising Na-CMC, step i), may comprise the ion exchange steps i) aa) and i) ab), and the reaction with spacer step i) c). The first step in the method of functionalising Na-CMC, step i), may comprise the activation step i) b) and the reaction with spacer step i) c). The first step in the method of functionalising Na-CMC, step i), may comprise the ion exchange step i) a), the activation step i) b), and the reaction with spacer step i) c). The first step in the method of functionalising Na-CMC, step i), may comprise the ion exchange steps i) aa) and i) ab), the activation step i) b), and the reaction with spacer step i) c).

### Step ii) Coupling of CMC-Spacer Compounds to form CMC-Reactive Member Compounds

The second step in the method of functionalising Na-CMC is coupling the CMC derivative comprising at least one unit of Formula 1 with a reactive member reagent to form a CMC derivative comprising at least one unit of Formula 2:

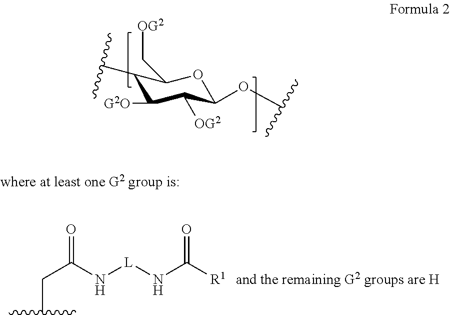

wherein R¹ is a reactive member. This step will be referred to as step ii).

In some embodiments, there is one at least one G² group in Formula 2. In some embodiments, the at least one G² group is on the C-6 hydroxyl group of Formula 2, such that the remaining C-2 and C-3 G² groups are H. In some embodiments, the at least one G² group is on the C-2 hydroxyl group of Formula 2, such that the remaining C-3 and C-6 G² groups are H. In some embodiments, the at least one G² group is on the C-3 hydroxyl group of Formula 2, such that the remaining C-2 and C-6 G² groups are H.

The group L is derived from the spacer in step i).

A CMC derivative comprising at least one unit of Formula 2 may be referred to as a CMC-reactive member compound.

A reactive member is a functional group or moiety which reacts in a click reaction, such as a SPAAC or Staudinger ligation. The reactive member reacts with a complementary reactive member in the click reaction. A reactive member for a SPAAC reaction may be a cyclooctyne derivative. A reactive member for a Staudinger ligation reaction may be a triaryl phosphine derivative with an electrophilic trap, such as the Staudinger ligand moiety, 3-(diphenylphosphino)-4-(methoxycarbonyl)-phenyl.

The reactive member reagent used in step ii) may be selected from:

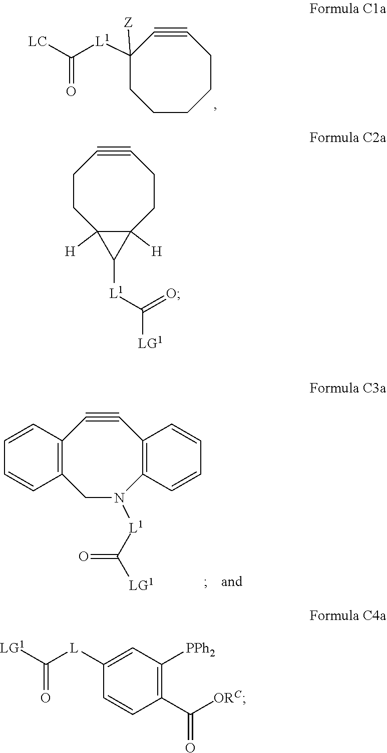

wherein LG¹ is a leaving group; L¹ is a linker group; Z is a halogen; and R^{C} is alkyl.

The leaving group LG¹ may be —OH or

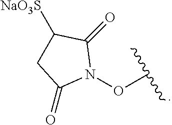

When the leaving group LG¹ is —OH, the —OH group may react with EDCI (1-ethyl-3-(3-dimethylaminopropyl) carbodiimide hydrochloride) to form LG¹ in situ under the reaction conditions of step ii):

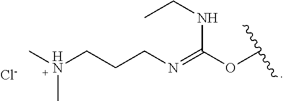

Preferably the leaving group LG¹ is

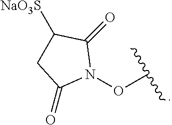

A linker group is a moiety that has two functional groups removed, such as —H. Each removed functional group is replaced with bond to another moiety.

The linker group L¹ may be a bond, alkylene, -(alkylene)O—*, —O(alkylene)—*, or -(alkylene)C(O)—* wherein the*is the attachment to the cyclooctyne or benzene ring. In some embodiments, the linker group L¹ is a bond. In some embodiments, the linker group L¹ is -(alkylene)O—*, such as -(methylene)O—* or —CH₂O—*. In some embodiments, the linker group L¹ is —O(alkylene)-*, such as —O(methylene)-* or —OCH₂-*. In some embodiments, the linker group L¹ is -(alkylene)C(O)—*, such as -(ethylene)C(O)—* or —CH₂CH₂C(O)—*

The linker group L¹ may be -(alkylene)O—*, wherein the * attachment to the cyclooctyne or benzene ring. The linker group L¹ may be —(C₁ alkylene)O—*, such as -(methylene)O—* or —CH₂O—*. The linker group L¹ may be —(C₂alkylene)O—*, such as-(ethylene)O—*. The linker group L¹ may be —(C₃ alkylene)O—*, such as -(n-propylene)O—*. The linker group L¹ may be —(C₄ alkylene)O—*, such as -(n-butylene)O—*. In some embodiments, the linker group L¹ is —(C₁-C₆ alkylene)O—*. In some embodiments, the linker group L¹ is —(C₁ alkylene)O—*, such as -(methylene)O—* or —CH₂O—*.

The linker group L¹ may be —O(alkylene)-*, wherein the * attachment to the cyclooctyne or benzene ring. The linker group L¹ may be —O(C₁ alkylene)-*, such as —O(methylene)-* or —OCH₂-*. The linker group L¹ may be —O(C₂ alkylene)-*, such as —O(ethylene)-*. The linker group L¹ may be —O(C₃ alkylene)-*, such as —O(n-propylene)-*. The linker group L¹ may be —O(C₄ alkylene)-*, such as —O(n-butylene)-*. In some embodiments, the linker group L¹ is —O(C₁-C₆ alkylene)-*. In some embodiments, the linker group L¹ is —O(C1 alkylene)-*, such as —O(methylene)-* or —OCH₂—*.

The linker group L¹ may be -(alkylene)C(O)—*, wherein the * attachment to the cyclooctyne or benzene ring. The linker group L¹ may be —(C₁ alkylene)C(O)—*, such as -(methylene)C(O)—* or —CH₂C(O)—*. The linker group L¹ may be —(C₂ alkylene)C(O)—*, such as -(ethylene)C(O)—*. The linker group L¹ may be —(C₃ alkylene)C(O)—*, such as -(n-propylene)C(O)—*. The linker group L¹ may be —(C₄ alkylene)C(O)—*, such as -(n-butylene)C(O)—*. In some embodiments, the linker group L¹ is —(C₁-C₆ alkylene)C(O)—*. In some embodiments, the linker group L¹ is —(C₂ alkylene)O—*, such as -(ethylene)C(O)—* or —CH₂CH2C(O)—*.

In some embodiments, the linker group L¹ is a bond.

The linker group L¹ may be an alkylene, such as C₁-C₆ alkylene. The linker group L¹ may be a C₁ alkylene, such as methylene. The linker group L¹ may be a C₂ alkylene, such as ethylene. The linker group L¹ may be a C₃ alkylene, such as n-propylene. The linker group L¹ may be a C₄ alkylene, such as n-butylene. In some embodiments, the linker group L¹ is C₁-C₆ alkylene. In some embodiments, the linker group L¹ is C₃ alkylene, such as n-propylene.

The Z group may be a halogen, such as —F, —Cl, —Br, or —I. In some embodiments, Z is —F.

The R^{C} group may be an alkyl, such as methyl (Me), ethyl (Et), or n-propyl (n-Pr). In some embodiments, R^{C} is Me.

The reactive member reagent used in step ii) of Formula C1a may be selected from:

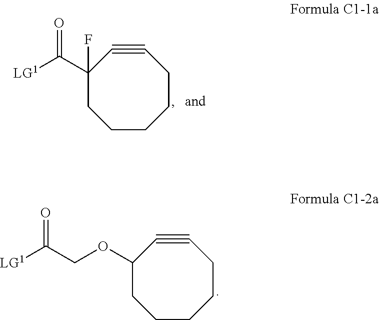

The reactive member reagent used in step ii) of Formula C2a may be:

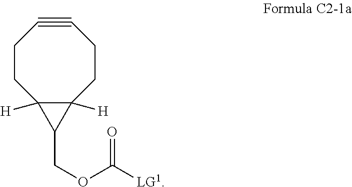

The reactive member reagent used in step ii) of Formula C3a may be:

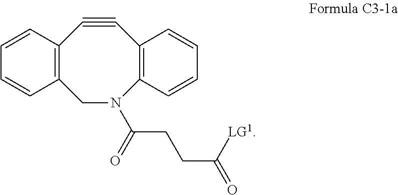

The reactive member reagent used in step ii) of Formula C4a may be:

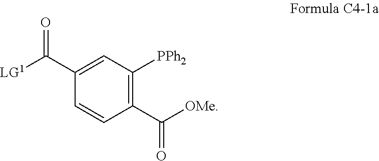

The reactive member R¹ is derived from the reactive member reagent used in step ii). The reactive member R¹ may be selected from:

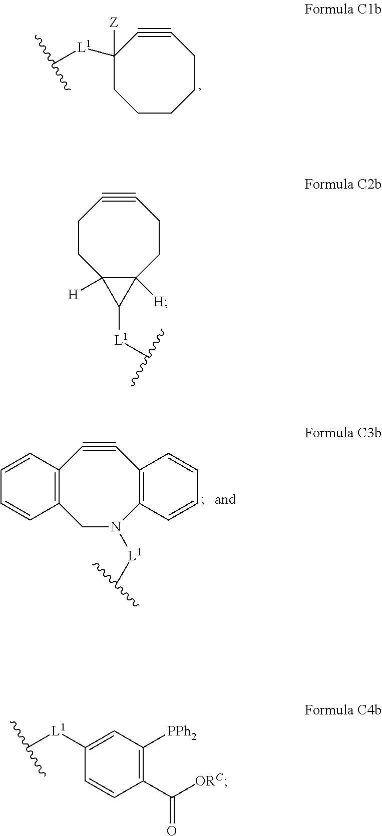

wherein L¹ is a linker group; Z is a halogen; and R^{C} is alkyl as discussed above. The linker group L¹, the Z group, and the R^{C} group have the same definition as defined for the reactive member reagent.

The reactive member R¹ of Formula C1b may be selected from:

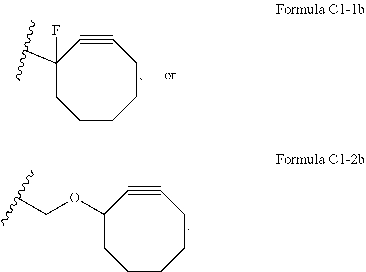 [SMILES: *C1(F)C#CCCCCC1 ; *COC1C#CCCCCC1]

The reactive member R¹ of Formula C2b may be:

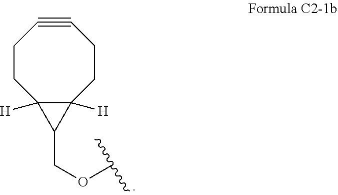 [SMILES: *OCC1C2CCC#CCCC21]

The reactive member R¹ of Formula C3b may be:

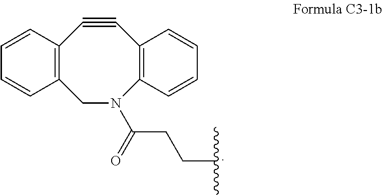 [SMILES: *CCC(=O)N1Cc2ccccc2C#Cc2ccccc21]

The reactive member R¹ of Formula C4b may be:

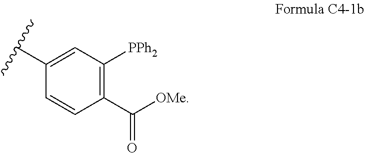

The R¹ reactive member Formula C1b is derived from the reactive member reagent Formula C1a used in step ii). The R¹ reactive member Formula C1-1b is derived from the reactive member reagent Formula C1-1a used in step ii). The R¹ reactive member Formula C1-2b is derived from the reactive member reagent Formula C1-2a used in step ii).

The R¹ reactive member Formula C2b is derived from the reactive member reagent Formula C2a used in step ii). The R¹ reactive member Formula C2-1b is derived from the reactive member reagent Formula C2-1a used in step ii).

The R¹ reactive member Formula C3b is derived from the reactive member reagent Formula C3a used in step ii). The R¹ reactive member Formula C3-1b is derived from the reactive member reagent Formula C3-1a used in step ii).

The R¹ reactive member Formula C4b is derived from the reactive member reagent Formula C4a used in step ii). The R¹ reactive member Formula C4-1b is derived from the reactive member reagent Formula C4-1a used in step ii).

The R¹ reactive member Formula C1b, such as Formula C1-1b and Formula C1-2b; Formula C2b, such as Formula C2-1b; and Formula C3b, such as Formula C3-1b, may be used in a SPAAC reaction with an azide functional group as the complementary reactive member in step iii). The R¹ reactive member Formula C4b, such as Formula C4-1b, may be used in a Staudinger ligation reaction with an azide functional group as the complementary reactive member in step iii).

In step ii), a coupling reagent may be added to facilitate the reaction, such as increasing the rate of reaction or coupling. The coupling reagent may be an aminium/uronium salt such as HBTU, HATU, TBTU, and HCTU. The coupling reagent may be a phosphonium salt such as PyBOP and PyAOP. In some embodiments, the coupling reagent is HBTU.

HBTU is hexafluorophosphate benzotriazole tetramethyl uronium or O-(benzotriazol-1-yl)-N,N,N′,N′-tetramethyluronium hexafluorophosphate or 2-(1H-benzotriazol-1-yl)-1, 1,3,3-tetramethyluronium hexafluorophosphate). HATU is hexafluorophosphate azabenzotriazole tetramethyl uronium or 1-[bis(dimethylamino)methylene]-1H-1,2,3-triazolo[4,5-b]pyridinium 3-oxide hexafluorophosphate. TBTU is tetrafluoroborate benzotriazole tetramethyl uronium or 2-(1H-benzotriazole-1-yl)-1,1,3,3-tetramethylaminium tetrafluoroborate. HCTU is 2-(6-chloro-1H-benzotriazole-1-yl)-1,1,3,3-tetramethylaminium hexafluorophosphate. PyBOP is benzotriazol-1-yloxytripyrrolidinophosphonium hexafluorophosphate. PyAOP is (7-azabenzotriazol-1-yloxy)tripyrrolidinophosphonium hexafluorophosphate.

In step ii), a base may be added to facilitate the reaction and act as a proton scavenger. The base used may be dry or anhydrous to prevent side reactions with water present. In some embodiments, the base is a tertiary amine, such as N,N-diisopropylethylamine (DIPEA) or triethylamine (Et₃N). In some embodiments, the base is N,N-diisopropylethylamine (DIPEA). Alternatively, the base may be a pyridine or a pyridine derivative, such as 2,6-lutidine, or 4-dimethylaminopyridine (DMAP).

In step ii), the reaction may be conducted at about room temperature.

In step ii), the reaction solvent may be a polar (hydrophilic) aprotic solvent. The reaction solvent may be N,N′-dimethylformamide (DMF), hexamethylphosphoramide (HMPA), acetonitrile (MeCN), or dimethylsulfoxide (DMSO). In some embodiments, the reaction solvent is N,N′-dimethylformamide (DMF).

The reaction solvent may be dry or anhydrous to prevent water from reacting with the reactive member reagent.

Alternatively, the reaction solvent in step ii) may be water or a buffer solution. In some embodiments, the reaction solvent may be a buffer solution selected from 2-(N-morpholino)ethanesulfonic acid (MES) buffer, 3-(N-morpholino)propanesulfonic acid (MOPS) buffer, 4-(2-hydroxyethyl)-1-piperazineethanesulfonic acid (HEPES) buffer, and phosphate-buffered saline (PBS). In some embodiments, the reaction solvent is phosphate-buffered saline (PBS).

The reaction of step ii) is conducted until there is no further change in the composition of the reaction mixture, such as when all of the CMC derivative comprising at least unit of Formula 1 has reacted with the reactive member reagent. In some embodiments, the reaction time may be at least 10 h, at least 12 h, at least 14 h, at least 16 h, at least 18 h, at least 20 h, at least 22 h, or at least 24 h. In some embodiments, the reaction time is overnight, such as at least 16 h.

The degree of substitution (DoS) of units of Formula 2 in the CMC derivative per D-anhydroglucopyranose monomers of cellulose may be quantified using elemental analysis, a ninhydrin assay (Kaiser Test), or 2,4,6-trinitrobenzenesulfonic acid (TNBS) assay. In some embodiments, the DoS of units of Formula 2 in the CMC derivative may be a range from about 0.15 to about 0.21, such as from about 0.17 to about 0.18. Typically, the DoS of units of Formula 2 in the CMC derivative is from about 0.17 to about 0.18.

Preferably, the DoS of units of Formula 2 in the CMC derivative is at least about 0.05, such as at least about 0.1, such as at least about 0.15.

A DoS of units of Formula 2 in the CMC derivative per D-anhydroglucopyranose monomers of cellulose for step ii) of from about 0.17 to about 0.18 corresponds to a DoC per available amines of a CMC derivative comprising at least one unit of Formula 1 of from about 0.66 to about 0.71.

The conversion between a CMC derivative comprising at least one unit of Formula 1 to a CMC derivative comprising at least one unit of Formula 2 may be from about 66% to about 71% conversion.

The reactive member reagent may be synthesised before use in step ii), as described in Examples 3, 4 and 5.

The skilled person will appreciate that the synthesis of the monosubstituted reactive member reagent of Formula C1-1a is afforded in three synthetic steps (Example 3) and the synthesis of the monosubstituted reactive member reagent of Formula C1-2a is afforded in three synthetic steps (Example 4). The syntheses of these cyclooctyne-derived reactive member reagents are significantly shorter in comparison to the known difluorosubstituted derivative DIFO (2-(2,2-difluorocyclooct-3-yn-1-yl)acetic acid), which has a synthetic pathway of ten steps (Codelli et al., 2008); dimethoxysubstituted aza derivative DIMAC (6,7-dimethoxyazacyclooct-4-yne), with a synthetic pathway of nine steps (Sletton et al., 2008); and biarylsubstituted aza derivative BARAC (biarylazacyclooctynone), with a synthetic pathway of eight steps (Jewett et al., 2010).

The skilled person will appreciate that the reactive member reagent of Formula C4a and the corresponding reactive member of Formula C4b may preferably be used, to prevent aza-ylide hydrolysis in the Staudinger ligation reaction (as described in step iii)). The ester (—COOR^{C}) is an electrophilic trap ortho to the phosphane to capture and stabilise the aza-ylide intermediate via intramolecular cyclisation to prevent hydrolysis of the aza-ylide. The reduction in aza-ylide hydrolysis prevents the azide group (as the complementary reactive member on the biomolecule described herein) from being reduced to an amine. Furthermore, the prevention of aza-ylide hydrolysis also improves the yield of the coupling reaction of the biomolecule to the CMC derivative.

### Step iii) Coupling of CMC-Reactive Member Compounds to form CMC-Biomolecule Compounds

The third step in the method of functionalising Na-CMC is coupling the CMC derivative comprising at least one unit of Formula 2 with a biomolecule comprising a complementary reactive member via a click reaction to form a CMC derivative product comprising at least one unit of Formula 3:

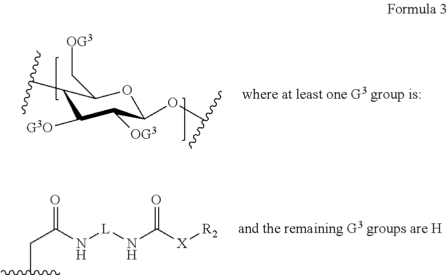

wherein X is a linker group derived from the click reaction between the reactive member R¹ of the CMC derivative comprising at least one unit of Formula 2 and the complementary reactive member on the biomolecule; and R² is derived from the biomolecule.

In some embodiments, there is one at least one G³ group in Formula 3. In some embodiments, the at least one G³ group is on the C-6 hydroxyl group of Formula 3, such that the remaining C-2 and C-3 G³ groups are H. In some embodiments, the at least one G³ group is on the C-2 hydroxyl group of Formula 3, such that the remaining C-3 and C-6 G³ groups are H. In some embodiments, the at least one G³ group is on the C-3 hydroxyl group of Formula 3, such that the remaining C-2 and C-6 G³ groups are H.

A complementary reactive member is a functional group or moiety which reacts in a click reaction, such as a SPAAC or Staudinger ligation. The complementary reactive member reacts with a reactive member in the click reaction. Preferably, the complementary reactive member is an azide functional group. When the complementary reactive member is an azide functional group, the click reaction may be a SPACC reaction or a Staudinger ligation.

The click reaction in step iii) occurs between the reactive member of the CMC derivative comprising at least one unit of Formula 2 and the complementary reactive member of the biomolecule. The type of click reaction in step iii) will depend on the reactive member R¹ of the CMC derivative comprising at least one unit of Formula 2 and the complementary reactive member of the biomolecule. The click reaction in step iii) may be a SPAAC reaction or a Staudinger ligation.

In some embodiments, the click reaction is a SPAAC reaction. When the click reaction is a SPAAC reaction, the reactive member R¹ may be Formula C1b, such as Formula C1-1b and Formula C1-2b; Formula C2b, such as Formula C2-1b; or Formula C3b, such as Formula C3-1b, and the complementary reactive member of the biomolecule is an azide functional group.

In some embodiments, the click reaction is a Staudinger ligation. When the click reaction is a Staudinger reaction, the reactive member R¹ may be Formula C4b and the complementary reactive member of the biomolecule is an azide functional group.

The linker group X is derived from the click reaction between the reactive member R¹ of the CMC derivative comprising at least one unit of Formula 2 and the complementary reactive member on the biomolecule. The click rection may be a SPAAC reaction or a Staudinger ligation. The click reaction performed will depend on the reactive member R¹ of the CMC derivative comprising at least one unit of Formula 2 and the complementary reactive member of the biomolecule.

The linker group X may be selected from:

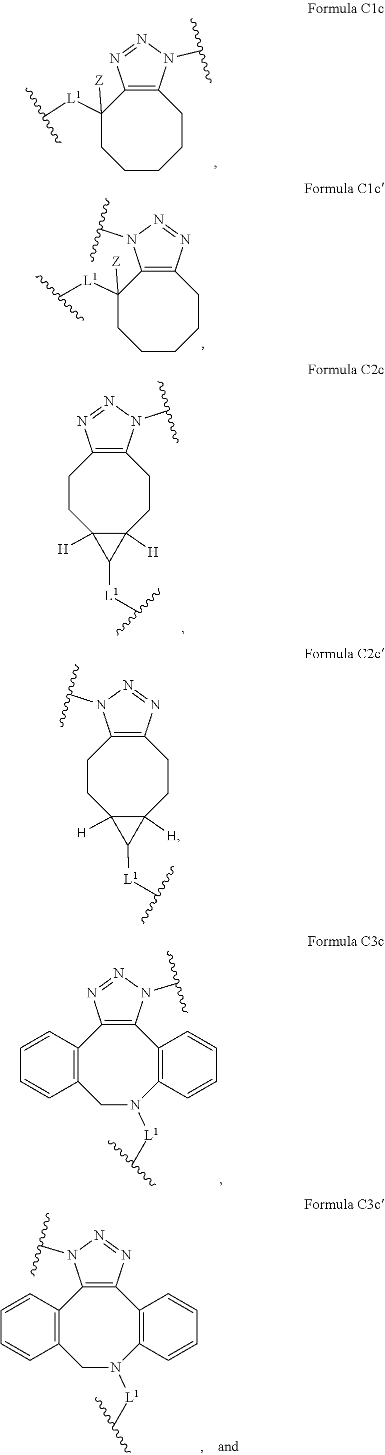

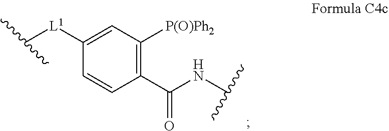

wherein L¹ is a linker group; Z is a halogen; and R^{C} is alkyl as discussed above. The linker group L¹, the Z group, and the R^{C} group have the same definition as defined for the reactive member reagent, but the*in linker group L¹ is the attachment to the cyclooctene or benzene ring.

Preferably, the linker group X is selected from Formula C1c, Formula C2c, Formula C3c, and Formula C4c.

The linker group X of Formula C1c or Formula C1c′ may be selected from:

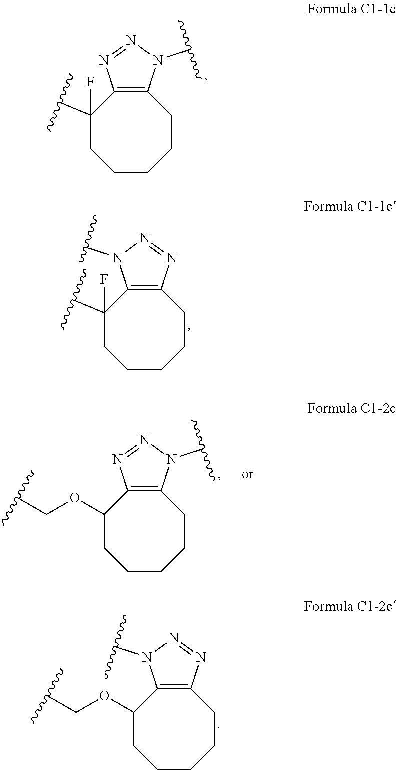 [SMILES: *n1nnc2c1CCCCCC2(*)F ; *n1nnc2c1C(*)(F)CCCCC2 ; *COC1CCCCCc2c1nnn2* ; *COC1CCCCCc2nnn(*)c21]

The linker group X of Formula C2c or Formula C2c′ may be:

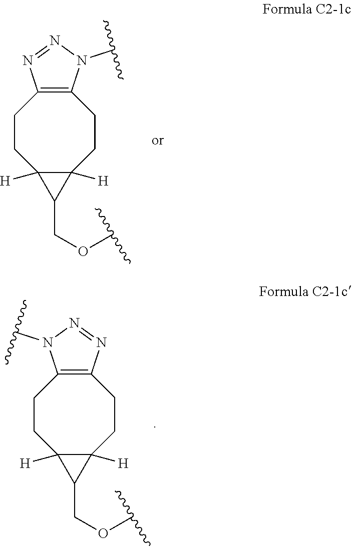 [SMILES: *OCC1C2CCc3nnn(*)c3CCC21 ; *OCC1C2CCc3nnn(*)c3CCC21]

The linker group X of Formula C3c or Formula C3c′ may be:

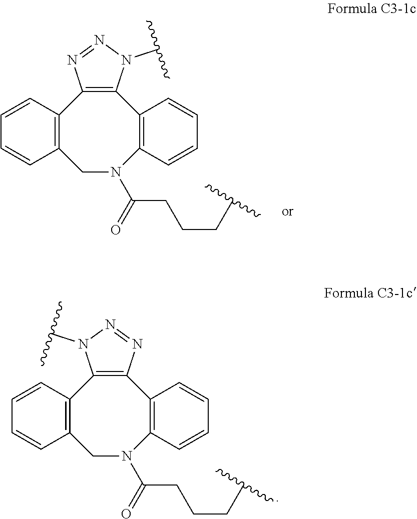 [SMILES: *CCCC(=O)N1Cc2ccccc2-c2nnn(*)c2-c2ccccc21 ; *CCCC(=O)N1Cc2ccccc2-c2c(nnn2*)-c2ccccc21]

The linker group X of Formula C4c may be:

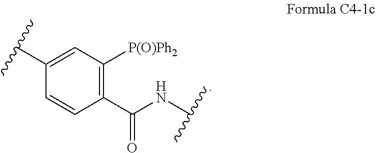

The X linker group of Formula C1c, such as Formula C1-1c and Formula C1-2c; Formula C1c′, such as Formula C1-1c′ and Formula C1-2c′; Formula C2c, such as Formula C2-1c; Formula C2c′, such as

Formula C2-1c′; Formula C3c, such as Formula C3-1c; Formula C3c′, such as Formula C3-1c′; and Formula C4c, such as Formula C4-1c, is derived from a biomolecule with an azide functional group as the complementary reactive member.

The X linker group of Formula C1c or Formula C1c′ is derived from the R¹ reactive member Formula C1b from the CMC derivative comprising at least one unit of Formula 2 used in step iii). The X linker group of Formula C1-1c is derived from the R¹ reactive member Formula C1-1b from the CMC derivative comprising at least one unit of Formula 2 used in step iii). The X linker group of Formula C1-2c is derived from the R¹ reactive member Formula C1-2b from the CMC derivative comprising at least one unit of Formula 2 used in step iii).

The X linker group of Formula C2c or Formula C2c′ is derived from the R¹ reactive member Formula C2b from the CMC derivative comprising at least one unit of Formula 2 used in step iii). The X linker group of Formula C2-1c is derived from the R¹ reactive member Formula C2-1b from the CMC derivative comprising at least one unit of Formula 2 used in step iii).

The X linker group of Formula C3c or Formula C3c′ is derived from the R¹ reactive member Formula C3b from the CMC derivative comprising at least one unit of Formula 2 used in step iii). The X linker group of Formula C3-1c is derived from the R¹ reactive member Formula C3-1b from the CMC derivative comprising at least one unit of Formula 2 used in step iii).

The X linker group of Formula C4c is derived from the R¹ reactive member Formula C4b from the CMC derivative comprising at least one unit of Formula 2 used in step iii). The X linker group of Formula C4-1c is derived from the R¹ reactive member Formula C4-1b from the CMC derivative comprising at least one unit of Formula 2 used in step iii).

The X linker group of Formula C1c, such as Formula C1-1c and Formula C1-2c; Formula C1c′, such as Formula C1-1c′ and Formula C1-2c′; Formula C2c, such as Formula C2-1c; Formula C2c′, such as Formula C2-1c′; Formula C3c, such as Formula C3-1c; and Formula C3c′, such as Formula C3-1c′, are derived from a SPAAC reaction with an azide functional group as the complementary reactive member in step iii). The X linker group of Formula C4c, such as Formula C4-1c, is derived from a Staudinger ligation reaction with an azide functional group as the complementary reactive member in step iii).

### Biomolecule Comprising a Complementary Reactive Member

The skilled person will appreciate that the biomolecule comprising a complementary reactive member for the click reaction in step iii) may be any biomolecule. The biomolecule may comprise a saccharide moiety, a metal chelator, a biological probe (such as a fluorescent probe), or a peptide.

The group R² is derived from the biomolecule used in step iii). The bond between the biomolecule and the complementary reactive member is converted to the bond between the group R² and the linker group X of the CMC derivative product comprising at least one unit of Formula 3 in the click reaction of step iii).

The biomolecule may be a compound of Formula B:

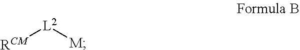

wherein R^{CM} is a complementary reactive member; L² is an alkylene, -(alkylene)C(O)—*, -(alkylene)NHC(O)—*, or a bond, wherein the*is the attachment to M; and M is a biomolecular unit.

The group R² may be —L²—M, wherein L² is an alkylene, -(alkylene)C(O)—*, -(alkylene)NHC(O)—*, —(CH₂CH₂O)ₘCH₂—*, —CH₂(OCH₂CH₂)ₘ-*, arylene, or a bond, wherein the*is the attachment to M and m is an integer between 1 to 450; and M is a biomolecular unit.

When the biomolecule is of Formula B, the group R² is-L2—M.

The linker group L² may be an alkylene, such as C₁-C₆ alkylene. In some embodiments, the linker group L² is C₁-C₆ alkylene. The linker group L² may be a C₁ alkylene, such as methylene. The linker group L² may be a C₂ alkylene, such as ethylene. The linker group L² may be a C₃ alkylene, such as n-propylene. The linker group L² may be a C₄ alkylene, such as n-butylene. In some embodiments, the linker group L² is C₃ alkylene, such as n-propylene.

The linker group L² may be -(alkylene)C(O)—*, wherein the*is the attachment to M. In some embodiments, the linker group L² is —(C₁-C₆ alkylene)C(O)—*. The linker group L² may be —(C₁ alkylene)C(O)—*, such as -(methylene)C(O)—* or —CH2C(O)—*. The linker group L² may be —(C₂ alkylene)C(O)—*, such as -(ethylene)C(O)—*. The linker group L² may be —(C₃ alkylene)C(O)—*, such as -(n-propylene) C(O)—*. The linker group L² may be —(C₄ alkylene)C(O)—*, such as -(n-butylene)C(O)—*. In some embodiments, the linker group L² is —(C₁ alkylene)C(O)—*, such as -(methylene)C(O)—* or —CH₂C(O)—*.

The linker group L² may be -(alkylene)NHC(O)—*, wherein the*is the attachment to M. In some embodiments, the linker group L² is —(C₁-C₆ alkylene)NHC(O)—*. The linker group L² may be —(C₁ alkylene)NHC(O)—*, such as -(methylene)NHC(O)—* or —CH2NHC(O)—*. The linker group L² may be —(C₂ alkylene)NHC(O)—*, such as -(ethylene)NHC(O)—*. The linker group L² may be —(C₃ alkylene) NHC(O)—*, such as -(n-propylene)NHC(O)—*. The linker group L² may be —(C₄ alkylene)NHC(O)—*, such as -(n-butylene)NHC(O)—*. In some embodiments, the linker group L² is —(C₃ alkylene)NHC(O)—*, such as -(n-propylene)NHC(O)—*.

The linker group L² may be —(CH₂CH₂O)ₘCH₂—*, wherein the*is the attachment to M and m is an integer from 1 to 450. Alternatively, the linker group L² may be —CH₂(OCH₂CH₂)ₘ—*, wherein the*is the attachment to M and m is an integer from 1 to 450. In some embodiments, m is an integer from 1 to 450. In some embodiments, m is an integer from 1 to 10. In some embodiments, m is an integer from 1 to 5. In some embodiments, m is an integer from 1 to 3. In some embodiments, m is an integer from 2 to 3. In some embodiments, m is 3. In some embodiments, n is 2. In some embodiments, m is 1. When referring to m, the range includes the lower and upper limits.

The linker group L² may be an arylene, such as C₆ arylene, such as phenylene. The attachments to R^{CM} and M may be at any carbons, such as C-1, C-2, C-3, C-4, C-5, or C-6 if present. In some embodiments, the attachments to R^{CM} and M may be at C-1 and C-4 to minimise steric hindrance.

In some embodiments, the linker group L² may be a bond.

Preferably, the complementary reactive member R^{CM} is an azide functional group (—N₃). In this embodiment, the biomolecule may be Formula B1:

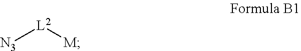

wherein N₃ is an azide functional group; L² is an alkylene, -(alkylene)C(O)—* or a bond, wherein the*is the attachment to M; and M is a biomolecular unit.

In some embodiments, the biomolecule may comprise a saccharide moiety, such as when the biomolecular unit M is a saccharide unit. In some embodiments, the biomolecule may comprise a metal chelator, such as when the biomolecular unit M is a metal chelator. In some embodiments, the biomolecule may comprise a fluorescent probe, such as when the biomolecular unit M is a fluorescent probe (fluorophore). In some embodiments, the biomolecule may comprise a peptide, such as when the biomolecular unit M is a peptide.

In some embodiments, the group R² may comprise a saccharide moiety, such as when the biomolecular unit M is a saccharide moiety. In some embodiments, the group R² may comprise a metal chelator, such as when the biomolecular unit M is a metal chelator. In some embodiments, the group R² may comprise a fluorescent probe, such as when the biomolecular unit M is a fluorescent probe (fluorophore). In some embodiments, the group R² may comprise a peptide, such as when the biomolecular unit M is a peptide.

### Biomolecular Unit M is a Saccharide Moiety

The biomolecule of Formula B, such as Formula B1, may comprise a saccharide moiety as biomolecular unit M. The group R² may comprise a saccharide moiety as biomolecular unit M.

A saccharide moiety (Sacc) is a saccharide wherein at least one of the functional groups, such as an —OH group or a —H from an —OH group, is removed and replaced with a bond to the linker group L². The functional group may be removed from any carbon of the saccharide, such as C-1, C-2, C-3, C-4, C-5 or C-6, if present. If the functional group is a —H from an —OH group, the —H may be removed from an —OH group attached to any carbon of the saccharide, such as C-1, C-2, C-3, C-4, C-5 or C-6, if present.

Preferably, the biomolecular unit M is a terminal saccharide moiety. A terminal saccharide moiety (-Sacc-H) is a saccharide with a single functional group, such as an —OH group or a —H from an —OH group, is removed and replaced with a bond to the linker group L². Preferably, when the biomolecular unit M is a terminal saccharide moiety, a —H from an —OH group is removed from the saccharide. The —H group may be removed from an-OH group attached to any carbon of the saccharide, such as C-1, C-2, C-3, C-4, C-5 or C-6, if present. Preferably, the-H group may be removed from the C-1 —OH group.

The saccharide moiety may be derived from any saccharide. In some embodiments, the saccharide may be a monosaccharide. In other embodiments, the saccharide may be a disaccharide, oligosaccharide, or a polysaccharide.

A disaccharide is a saccharide made of 2 monosaccharide units joined together by a glycosidic bond. An oligosaccharide is a saccharide made of 2 to 11 monosaccharide units joined together by glycosidic bonds. A polysaccharide is a saccharide made of 12 or more monosaccharide units joined together by glycosidic bonds. A monosaccharide unit is a monosaccharide wherein at least one of the —OH groups is used to form a glycosidic bond.

A glycosidic bond is a covalent bond formed in a condensation reaction that joins the C-1 carbon of one monosaccharide unit to another atom of another compound, typically another monosaccharide. Typically, the atom is an oxygen atom (O), such as the C-2, C-3, C-4, C-5, or C-6 oxygen atom if present, of the other monosaccharide to form an O-glycosidic bond. Typically, the atom is the C-4 oxygen atom of the other monosaccharide to form a 1,4-O-glycosidic bond. In other embodiments, the atom is a nitrogen atom (N) to form an N-glycosidic bond; sulfur atom(S) to form a S-glycosidic bond; or carbon atom (C) to form a C-glycosidic bond.

Each glycosidic bond may be an α- or β-glycosidic bond. An α-glycosidic bond is when the atom forming the glycosidic bond (such as an oxygen atom) attached to the C-1 carbon is on the opposite relative face of the saccharide when represented as a Haworth projection as the substituent at the other carbon attached to the ring oxygen of the same saccharide (such as C-5 in a hexose), whereas a β-glycosidic bond is when the atom forming the glycosidic bond (such as an oxygen atom) attached to the C-1 carbon is on the same relative face of the saccharide when represented as a Haworth projection as the substituent at the other carbon attached to the ring oxygen of the same saccharide (such as C-5 in a hexose), for example:

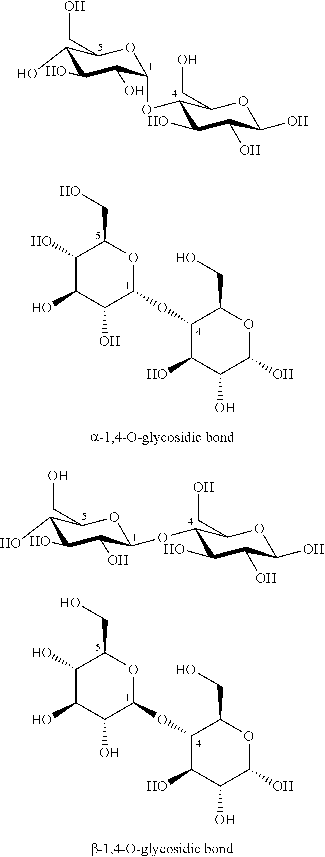

Preferably, the saccharide is a monosaccharide. In some embodiments, the saccharide may be a 6-membered pyranose or 5-membered furanose monosaccharide.

The saccharide may be D-or L-saccharide. Preferably, the saccharide is a D-saccharide. The saccharide may be the α- or β-anomer at C-1, or present as a mixture of anomers. The α-anomer is when the —OH attached to the C-1 carbon is on the opposite relative face of the saccharide when represented as a Haworth projection as the substituent at the other carbon attached to the ring oxygen of the same saccharide (such as C-5 in a hexose). The β-anomer is when the —OH attached to the C-1 carbon is on the same relative face of the saccharide when represented as a Haworth projection as the substituent at the other carbon attached to the ring oxygen of the same saccharide (such as C-5 in a hexose). When α- or β- is not mentioned, the saccharide is a mixture of anomers.

In some embodiments, the saccharide is a 5-membered furanose monosaccharide. In some embodiments, the saccharide may be a 5-membered furanose monosaccharide such as D-ribose (Ribf) or D-arabinose (Araf) (Table 1). Any of the —OH groups at C-1 to C-5 may be replaced by —H; a halogen (such as —F, —Cl, —Br, and —I); —OR^{O}; —C(O)alkyl; —C(O)OR^{O}, —SR^{S}, —NR_{N}²; wherein R^{O} is alkyl, aryl, acyl (such as acetyl or Ac or —C(O)CH₃), —S(O)₂OX, or —P(O)(OX)₂; R^{S} is H, alkyl or aryl; each R^{N} is independently H, alkyl or acyl (such as acetyl or Ac or —C(O)CH₃); and X is H, Na or K. Any of the carbon atoms, such as C-1, C-2, C-3, C-4, or C-5, may be ¹³C labelled.

TABLE 1
Structures of 5-membered furanose monosaccharides

| | Haworth projection | Mills projection |
|---|---|---|
| α-Ribf | 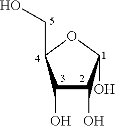 | 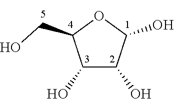 |
| β-Ribf | 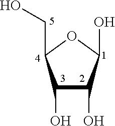 | 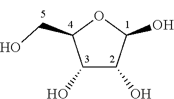 |
| α-Araf | 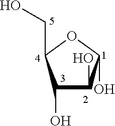 | 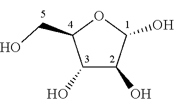 |
| β-Araf | 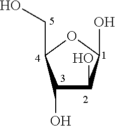 | 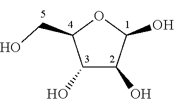 |

Preferably, the saccharide is a 6-membered pyranose monosaccharide. In some embodiments, the saccharide may be a 6-membered pyranose monosaccharide such as D-glucose (Glc), D-galactose (Gal), or D-mannose (Man) (Table 2). Any of the-OH groups at C-1 to C-5 may be replaced by —H; a halogen (such as —F, —Cl, —Br, and —I); —OR^{O}; —C(O)alkyl; —C(O)OR^{O}, —SR^{S}, —NR^{N}₂; wherein R^{O} is alkyl, aryl, acyl (such as acetyl or Ac or —C(O)CH₃), —S(O)₂OX, or —P(O)(OX)₂; R^{S} is H, alkyl or aryl; each R^{N} is independently H, alkyl or acyl (such as acetyl or Ac or —C(O)CH₃); and X is H, Na or K. Any of the carbon atoms, such as C-1, C-2, C-3, C-4, or C-5, may be ¹³C labelled.

TABLE 2
Structures of 6-membered pyranose monosaccharides

| | Chair conformation | Mills projection |
|---|---|---|
| α-Glc | 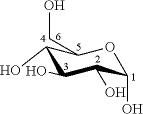 | 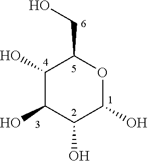 [SMILES: OC[C@H]1O[C@H](O)[C@H](O)[C@@H](O)[C@@H]1O] |
| β-Glc | 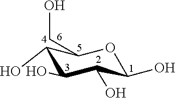 | 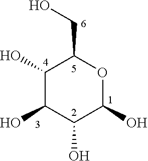 [SMILES: OC[C@H]1O[C@@H](O)[C@H](O)[C@@H](O)[C@@H]1O] |
| α-Gal | 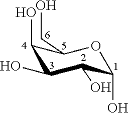 | 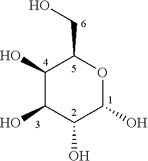 [SMILES: OC[C@H]1O[C@H](O)[C@H](O)[C@@H](O)[C@H]1O] |
| β-Gal | 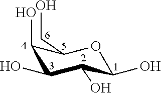 | 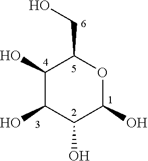 [SMILES: OC[C@H]1O[C@@H](O)[C@H](O)[C@@H](O)[C@H]1O] |
| α-Man | 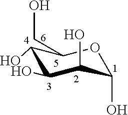 | 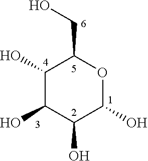 [SMILES: OC[C@H]1O[C@H](O)[C@@H](O)[C@@H](O)[C@@H]1O] |
| β-Man | 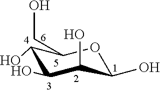 | 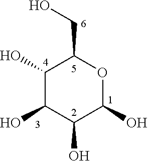 [SMILES: OC[C@H]1O[C@@H](O)[C@@H](O)[C@@H](O)[C@@H]1O] |

In some embodiments, the saccharide may be an amino sugar, wherein an —OH group of a 6-membered pyranose monosaccharide is replaced by —NR^{N}₂; wherein each R^{N} is independently H, alkyl or acyl (such as acetyl or Ac or —C(O)CH3). Examples of an amino monosaccharide in a 6-membered pyranose ring are glucosamine (GlcN) and N-acetylglucosamine (GlcNAc) where the C-2 —OH of D-glucose (Glc) is replaced by —NH₂ and —NHAc respectively (Table 3). Other examples of amino monosaccharides in a 6-membered pyranose ring are mannosamine (ManN) and N-acetylmannosamine (ManNAc) where the C-2 —OH of D-mannose (Man) is replaced by —NH₂ and —NHAc respectively (Table 3). Another example of a 6-membered pyranose monosaccharide amino saccharide is the peracetylated N-acetylmannosamine (Man(NAc)Ac₄) where the C-2 —OH of D-mannose (Man) is replaced by —NHAc and the C-1, C-3, C-4 and C-6 —OH groups are replaced by acetate (—OAc or —OC(O)CH₃) groups (Table 3). Peracetylated N-acetylmannosamine (Man(NAc)Ac₄) may also be considered a derivative of N-acetylmannosamine (ManNAc).

TABLE 3
Structures of 6-membered pyranose monosaccharides

| | Chair conformation | Mills projection |
|---|---|---|
| α-GlcN | 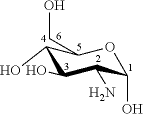 | 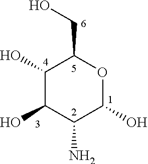 [SMILES: N[C@@H]1[C@@H](O)[C@H](O)[C@@H](CO)O[C@@H]1O] |
| β-GlCN | 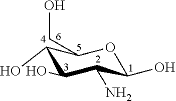 | 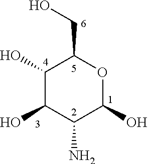 [SMILES: N[C@@H]1[C@@H](O)[C@H](O)[C@@H](CO)O[C@H]1O] |
| α-GlcNAc | 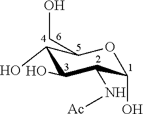 | 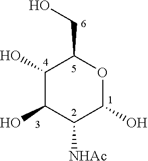 [SMILES: CC(=O)N[C@@H]1[C@@H](O)[C@H](O)[C@@H](CO)O[C@@H]1O] |
| β-GlcNAc | 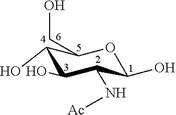 | 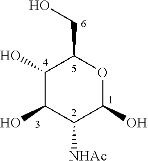 [SMILES: CC(=O)N[C@@H]1[C@@H](O)[C@H](O)[C@@H](CO)O[C@H]1O] |
| α-ManN | 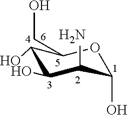 | 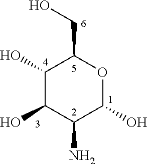 [SMILES: N[C@H]1[C@@H](O)[C@H](O)[C@@H](CO)O[C@@H]1O] |
| β-ManN | 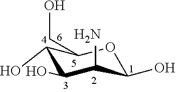 | 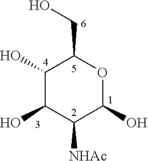 [SMILES: CC(=O)N[C@H]1[C@@H](O)[C@H](O)[C@@H](CO)O[C@H]1O] |
| α-ManNAc | 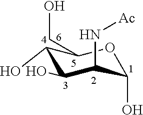 | 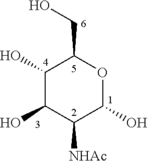 [SMILES: CC(=O)N[C@H]1[C@@H](O)[C@H](O)[C@@H](CO)O[C@@H]1O] |
| β-ManNAc | 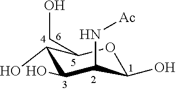 | 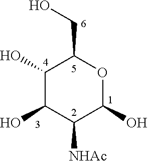 [SMILES: CC(=O)N[C@H]1[C@@H](O)[C@H](O)[C@@H](CO)O[C@H]1O] |
| α-Man(NAc)Ac₄ | 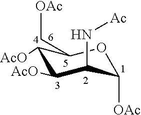 | 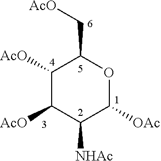 [SMILES: CC(=O)N[C@@H]1[C@@H](OC(C)=O)O[C@H](COC(C)=O)[C@@H](OC(C)=O)[C@@H]1OC(C)=O] |
| β-Man(NAc)Ac₄ | 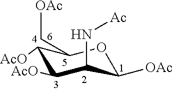 | 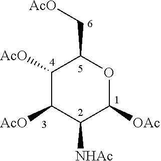 [SMILES: CC(=O)N[C@@H]1[C@H](OC(C)=O)O[C@H](COC(C)=O)[C@@H](OC(C)=O)[C@@H]1OC(C)=O] |

In some embodiments, the saccharide moiety for the biomolecular unit M is derived from a 6-membered pyranose monosaccharide such as N-acetylglucosamine (GlcNAc), N-acetylmannosamine (ManNAc) or peracetylated N-acetylmannosamine (Man(NAc)Ac₄). In some embodiments, the saccharide moiety for the biomolecular unit M is derived from N-acetylglucosamine (GlcNAc).

When the biomolecular unit M is a saccharide moiety, the saccharide moiety may be connected to the linker group L² via the C-1 oxygen atom:

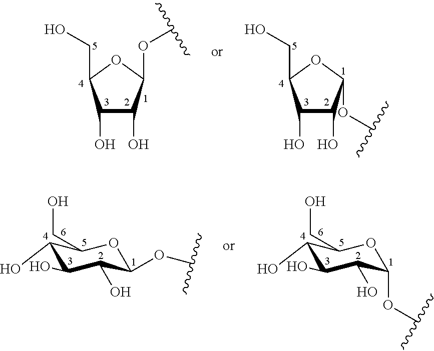

wherein any of the —OH groups at C-1 to C-5 or C-6 if present may be replaced by —H; a halogen (such as —F, —Cl, —Br, and —I); —ORº; —C(O) alkyl; —C(O)OR^{O}, —SR^{S}, —NR^{N}₂; wherein R^{O} is alkyl, aryl, acyl (such as acetyl or Ac or —C(O)CH₃), —S(O)₂OX, or —P(O)(OX)₂; R^{S} is H, alkyl or aryl; each R^{N} is independently H, alkyl or acyl (such as acetyl or Ac or —C(O)CH₃); and X is H, Na or K. Any of the carbon atoms, such as C-1, C-2, C-3, C-4, or C-5, or C-6 if present, may be ¹³C labelled.

When the biomolecular unit M is a saccharide moiety connected to the linker group L² via the C-1 oxygen atom, the saccharide moiety for the biomolecular unit M may be:

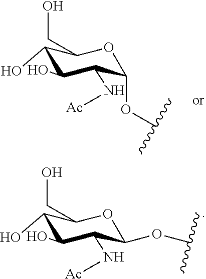

When the biomolecular unit M is a saccharide moiety connected to the linker group L² via the C-1 oxygen atom, the linker group L² is preferably an alkylene group. In some embodiments, the linker group L² is preferably a C₁-C₆ alkylene group. In some embodiments, the linker group L² is C₃ alkylene, such as n-propylene. Preferably, the linker group L² is attached to the C-1 oxygen of the saccharide moiety, derived from the removal of the —H of the —OH group on the C-1 carbon of the saccharide.

When the biomolecular unit M is a saccharide moiety connected to the linker group L² via the C-1 oxygen atom, the group R² may be:

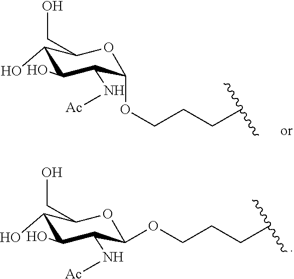

Alternatively, when the biomolecular unit M is a saccharide moiety, the saccharide moiety may be connected to the linker group L² via a group that has substituted an —OH group of the saccharide. An example may be where a —H group is removed from the acetyl (Ac or —C(O)CH₃) group of the —NHAc group (the group that has substituted an —OH group of the saccharide) of N-acetylglucosamine (GlcNAc) or N-acetylmannosamine (ManNAc):

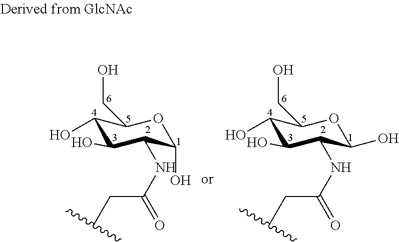

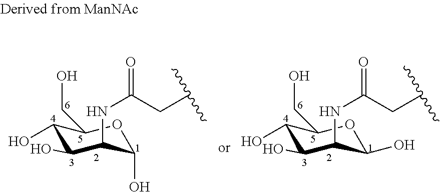

Alternatively, when the biomolecular unit M is a saccharide moiety, the saccharide moiety may be directly connected to the linker group L² by a carbon of the saccharide. The carbon may be any carbon of the saccharide, such as C-1, C-2, C-3, C-4, C-5 or C-6, if present. Examples may be where an —OH group is removed from C-1, C-2, or C-6 of D-glucose (Glc), wherein the stereochemistry at C-1 may be up or down, or α or β:

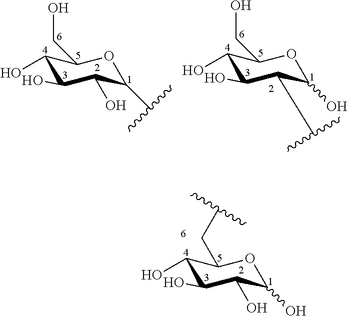

When the biomolecular unit M is a saccharide moiety connected to the linker group L² via a group that has substituted an —OH group of the saccharide or directly to a carbon of the saccharide, the linker group L² is preferably a bond.

When the biomolecular unit M is a saccharide moiety in step iii), the reaction may be conducted at about room temperature.

When the biomolecular unit M is a saccharide moiety in step iii), the reaction solvent may be an alcohol solvent. In some embodiments, the reaction solvent may be selected from methanol, ethanol, n-propanol, iso-propanol, n-butanol, sec-butanol, iso-butanol, tert-butanol, or benzyl alcohol. In some embodiments, the reaction solvent is methanol.

Alternatively, when the biomolecular unit M is a saccharide moiety in step iii), the reaction solvent may be a polar (hydrophilic) aprotic solvent, such as tetrahydrofuran (THF), N,N′-dimethylformamide (DMF), hexamethylphosphoramide (HMPA), or dimethylsulfoxide (DMSO).

Alternatively, when the biomolecular unit M is a saccharide moiety in step iii), the reaction solvent may be water or a buffer solution. In some embodiments, the reaction solvent may be a buffer solution selected from 2-(N-morpholino)ethanesulfonic acid (MES) buffer, 3-(N-morpholino)propanesulfonic acid (MOPS) buffer, 4-(2-hydroxyethyl)-1-piperazineethanesulfonic acid (HEPES) buffer, and phosphate-buffered saline (PBS).

Alternatively, when the biomolecular unit M is a saccharide moiety in step iii), the reaction solvent may be mixture of any of the reaction solvents described above. The mixture of reaction solvents may be a polar (hydrophilic) aprotic solvent and water. In some embodiments, the mixture of reaction solvents may be tetrahydrofuran (THF) and water. The ratio of the mixture of reaction solvents of polar (hydrophilic) aprotic solvent:water may be 1:1, 2:1, 3:1 or 4:1. In some embodiments, the mixture of reaction solvents is tetrahydrofuran (THF):water in the ratio 3:1.

When the biomolecular unit M is a saccharide moiety, the reaction of step iii) is conducted until there is no further change in the composition of the reaction mixture, such as when all of the CMC derivative comprising at least one unit of Formula 2 has reacted with the biomolecule comprising the complementary reactive member. In some embodiments, the reaction time may be at least 30 min, at least 1 h, at least 1 h and 30 min, or at least 2 h. In some embodiments, the reaction time at least 2 h.

The inventors envisage that when the CMC derivative product comprising at least one unit of Formula 3 comprises a saccharide moiety (such as the biomolecular unit M is a saccharide moiety), the saccharide moiety may be further functionalised either chemically or biochemically.

Saccharide-based hydrogels, such as CMC derivative products comprising a saccharide moiety as described herein, may influence cell behaviour by containing native cellular binding motifs (similar to the ECM) and soluble signalling molecules (Kharkar et al., 2013). The saccharide moiety may be able to interact with glycoproteins in the extracellular matrix (ECM), making these CMC derivative products promising materials for tissue engineering, including wound dressings (Francesko et al., 2011; Shelke et al., 2014).

In particular, saccharide moieties derived from N-acetylglucosamine (GlcNAc) or N-acetylmannosamine (ManNAc) (such as N-acetylmannosamine, Man(NAc)Ac₄) may be used as N-acetylneuraminic acid (Neu5Ac) precursors. Neu5Ac is the most abundant form of sialic acid. The biosynthesis of Neu5Ac has been shown to use GlcNAc and ManNAc, and the Neu5Ac may be biosynthetically introduced into the cell surface glycans of living cells (Keppler et al., 2001). This metabolic oligosaccharide engineering may be applied to several biological applications, such as artificial cellular receptor generation and cell surface labelling (Saxon et al., 2000a; Laughlin et al., 2006).

Furthermore, unnatural azidosaccharides (derived from Man(NAc)Ac₄, such as Az-Man(NAc)Ac₄ where the azide functional group (Az) is attached to the acetyl (Ac) group on —NHAc on the C-2 of the saccharide) may be biosynthetically metabolised onto the surface of glycans in human dermal fibroblasts (Saxon et al., 2000a). These bioengineered cells may also be subsequently coupled to the active CMC surfaces to provide a potentially tissue-mimetic surface, akin to tissue engineered skin substitute such as Apligraf® (Falanga et al., 1999).

Additionally, the CMC derivative products comprising a saccharide moiety as described herein may also have anti-cancer applications.

### Biomolecular Unit M is a Metal Chelator

The biomolecule of Formula B, such as Formula B1, may comprise a metal chelator as biomolecular unit M. The group R² may comprise a metal chelator as biomolecular unit M.

A metal chelator is a functional group or moiety that is able to chelate metal ions, such as metal cations. The functional group or moiety may comprise heteroatoms, such as oxygen (O) atoms or nitrogen (N) atoms. Preferably, the metal chelator comprises nitrogen (N) atoms. Examples of known metal chelators include ethylenediaminetetraacetic acid (EDTA) and tris(2-pyridylmethyl)amine (TPA).

The skilled person will appreciate that the metal cations may be derived from any metal atom. The metal atom may be an alkali metal (group I), alkaline earth metal (group II), a transition metal, a post-transition metal, a lanthanide, or an actinide. The metal atom may be Li, Na, K, Be, Mg, Ca, Sc, Ti, V, Cr, Mn, Fe, Co, Ni, Cu, Zn, Y, Zr, Nb, Mo, Tc, Ru, Rh, Pd, Ag, Cd, Al, Ga, In, Sn, or Pb. The metal cations may be Lit, Nat, K⁺, Mg²⁺, Ca²⁺, Sc³⁺, Cr³⁺, Fe²⁺, Fe³⁺, Ni²⁺, Cu⁺, Cu²⁺, Ag⁺, Pb²⁺, and Zn²⁺. Preferably, the metal cation is Zn²⁺ (or Zn(II)).

When the biomolecular unit M is a metal chelator, the metal chelator may be Formula M1 or Formula M2:

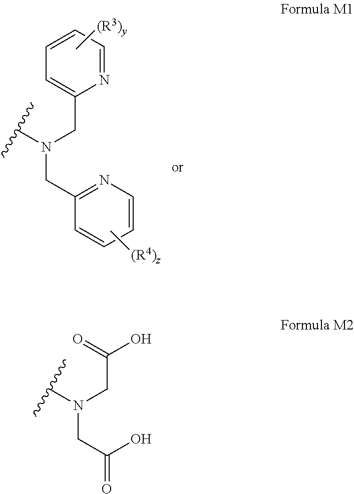

wherein y and z are each independently an integer from 0 to 4; and each R³ and R⁴ is independently selected from a halogen (such as —F, —Cl, —Br, and —I), —NO₂, —CO₂H, —CO₂(alkyl), —CN, alkyl, aryl, and acyl (such as acetyl or Ac or —C(O)CH₃); or, if one or both of y and z is an integer from 2 to 4, two R³ groups or two R⁴ groups on adjacent carbon atoms may be linked to form a fused benzene ring.

In some embodiments, the metal chelator for the biomolecular unit M is Formula M1. In some embodiments, the metal chelator for the biomolecular unit M is Formula M2.

In some embodiments, y is 0, 1, 2, 3, or 4. In some embodiments, y is 0. In some embodiments, y is 1. In some embodiments, y is 2. In some embodiments, y is 3. In some embodiments, y is 4. Preferably, y is 0, 1, or 2.

In some embodiments, z is 0, 1, 2, 3, or 4. In some embodiments, z is 0. In some embodiments, z is 1. In some embodiments, z is 2. In some embodiments, z is 3. In some embodiments, z is 4. Preferably, z is 0, 1, or 2.

In some embodiments, both y and z are 0. In some embodiments, both y and z are 1. In some embodiments, both y and z are 2.

When y is non-zero (as in 1, 2, 3, or 4), R³ is present. When y is greater than 1 (as in 2, 3, or 4), each R³ may be the same or different. In some embodiments, R³ is —F. In some embodiments, R³ is —Cl. In some embodiments, R³ is —Br. In some embodiments, R³ is —I. In some embodiments, R³ is —NO₂. In some embodiments, R³ is —CO₂H. In some embodiments, R³ is —CO₂(alkyl), such as —CO₂Me. In some embodiments, R³ is —CN. In some embodiments, R³ is alkyl, such as methyl. In some embodiments, R³ is aryl, such as phenyl. In some embodiments, R³ is acyl, such as acetyl (Ac or —C(O)CH₃). In some embodiments when y is an integer from 2 to 4, two R³ groups on adjacent carbon atoms are linked form a fused benzene ring. Preferably, R³ is —F or two R³ groups on adjacent carbon atoms are linked form a fused benzene ring if R³ is present (or when y is non-zero).

When z is non-zero (as in 1, 2, 3, or 4), R⁴ is present. When z is greater than 1 (as in 2, 3, or 4), each R⁴ may be the same or different. In some embodiments, R⁴ is —F. In some embodiments, R⁴ is —Cl. In some embodiments, R⁴ is —Br. In some embodiments, R⁴ is —I. In some embodiments, R⁴ is —NO₂. In some embodiments, R⁴ is —CO₂H. In some embodiments, R⁴ is —CO₂(alkyl), such as —CO₂Me. In some embodiments, R⁴ is —CN. In some embodiments, R⁴ is alkyl, such as methyl. In some embodiments, R⁴ is aryl, such as phenyl. In some embodiments, R⁴ is acyl, such as acetyl (Ac or —C(O)CH₃). In some embodiments when z is an integer from 2 to 4, two R⁴ groups on adjacent carbon atoms are linked to form a fused benzene ring. Preferably, R⁴ is —F or two R⁴ groups on adjacent carbon atoms are linked to form a fused benzene ring if R⁴ is present (or when z is non-zero).

In some embodiments when both y and z are non-zero (as in 1, 2, 3, or 4), each R³ and R⁴ is —F. In some embodiments when both y and z is an integer from 2 to 4, preferably when both y and z are 2, two R³ groups on adjacent carbon atoms are linked to form a fused benzene ring and two R⁴ groups on adjacent carbon atoms are linked to form a fused benzene ring.

When R³ is present, R³ may be connected to any one of the carbon atoms of the pyridinyl ring where there is a —H, such as C-3, C-4, C-5 or C-6. In some embodiments, R³ may be connected to the C-3 carbon. In some embodiments, R³ may be connected to the C-4 carbon. In some embodiments, R³ may be connected to the C-5 carbon. In some embodiments, R³ may be connected to the C-6 carbon. Preferably, R³ is connected to the C-5 carbon.

When R⁴ is present, R⁴ may be connected to any one of the carbon atoms of the pyridinyl ring where there is a —H, such as C-3, C-4, C-5 or C-6. In some embodiments, R⁴ may be connected to the C-3 carbon. In some embodiments, R⁴ may be connected to the C-4 carbon. In some embodiments, R⁴ may be connected to the C-5 carbon. In some embodiments, R⁴ may be connected to the C-6 carbon. Preferably, R⁴ is connected to the C-5 carbon.

When two R³ groups on adjacent carbon atoms are linked to form a fused benzene ring, the two R³ groups may be connected to any two adjacent carbon atoms of the pyridinyl ring where there is a —H, such as C-3 and C-4; C-4 and C-5; or C-5 and C-6. In some embodiments, the two R³ groups may be connected to the C-3 and C-4 carbon atoms and are linked to form a fused benzene ring. In some embodiments, the two R³ groups may be connected to the C-4 and C-5 carbon atoms and are linked to form a fused benzene ring. In some embodiments, the two R³ groups may be connected to the C-5 and C-6 carbon atoms and are linked to form a fused benzene ring. Preferably when two R³ groups on adjacent carbon atoms are linked to form a fused benzene ring, the two R³ groups are connected to C-5 and C-6 carbon atoms.

When two R⁴ groups on adjacent carbon atoms are linked to form a fused benzene ring, the two R⁴ groups may be connected to any two adjacent carbon atoms of the pyridinyl ring where there is a —H, such as C-3 and C-4; C-4 and C-5; or C-5 and C-6. In some embodiments, the two R⁴ groups may be connected to the C-3 and C-4 carbon atoms and are linked to form a fused benzene ring. In some embodiments, the two R⁴ groups may be connected to the C-4 and C-5 carbon atoms and are linked to form a fused benzene ring. In some embodiments, the two R⁴ groups may be connected to the C-5 and C-6 carbon atoms and are linked to form a fused benzene ring. Preferably when two R⁴ groups on adjacent carbon atoms are linked to form a fused benzene ring, the two R⁴ groups are connected to C-5 and C-6 carbon atoms.

In some embodiments, the metal chelator for the biomolecular unit M is:

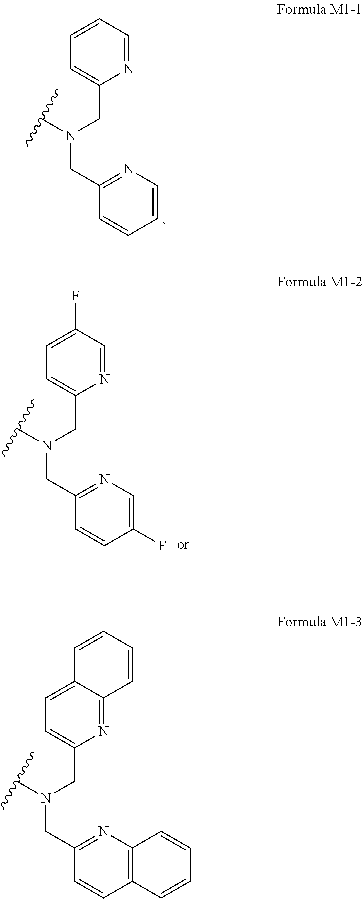 [SMILES: *N(Cc1ccccn1)Cc1ccccn1 ; *N(Cc1ccc(F)cn1)Cc1ccc(F)cn1 ; "or" ; *N(Cc1ccc2ccccc2n1)Cc1ccc2ccccc2n1]

In some embodiments, the metal chelator for the biomolecular unit M is Formula M1-1. In some embodiments, the metal chelator for the biomolecular unit M is Formula M1-2. In some embodiments, the metal chelator for the biomolecular unit M is Formula M1-3.

When the biomolecular unit M is a metal chelator connected to the linker group L², the linker group L² is preferably an alkylene group. In some embodiments, the linker group L² is preferably a C₁-C₆ alkylene group. In some embodiments, the linker group L² is C₃ alkylene, such as n-propylene.

When the biomolecular unit M is a metal chelator, the group R² may be Formula M1-1a, Formula M1-2a, or Formula M1-3a:

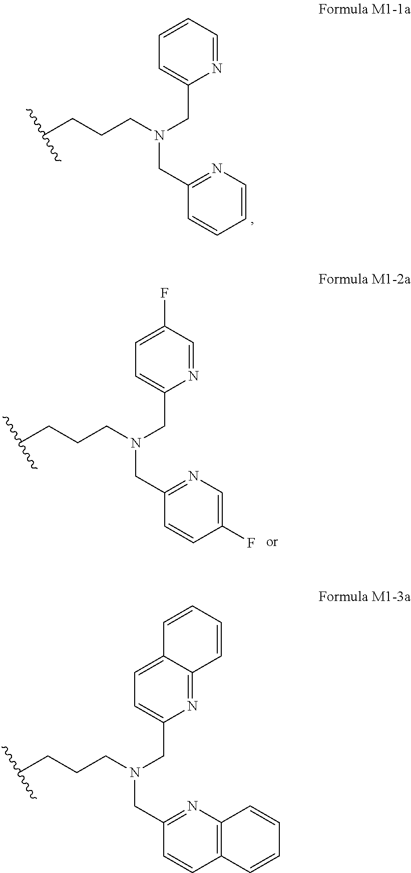 [SMILES: *CCCN(Cc1ccccn1)Cc1ccccn1 ; *CCCN(Cc1ccc(F)cn1)Cc1ccc(F)cn1 ; "or" ; *CCCN(Cc1ccc2ccccc2n1)Cc1ccc2ccccc2n1]

In some embodiments, the group R² may be Formula M1-1a. In some embodiments, the group R² may be Formula M1-2a. In some embodiments, the group R² may be Formula M1-3a.

When the biomolecular unit M is a metal chelator in step iii), the reaction may be conducted at about room temperature.

When the biomolecular unit M is a metal chelator in step iii), the reaction solvent may be an alcohol solvent. In some embodiments, the reaction solvent may be selected from methanol, ethanol, n-propanol, iso-propanol, n-butanol, sec-butanol, iso-butanol, tert-butanol, or benzyl alcohol. In some embodiments, the reaction solvent is methanol.

Alternatively, when the biomolecular unit M is a metal chelator in step iii), the reaction solvent may be a polar (hydrophilic) aprotic solvent, such as hexamethylphosphoramide (HMPA).

Alternatively, when the biomolecular unit M is a metal chelator in step iii), the reaction solvent may be water.

Alternatively, when the biomolecular unit M is a metal chelator in step iii), the reaction solvent may be mixture of any of the reaction solvents described above.

When the biomolecular unit M is a metal chelator, the reaction of step iii) is conducted until there is no further change in the composition of the reaction mixture, such as when all of the CMC derivative comprising at least one unit of Formula 2 has reacted with the biomolecule comprising the complementary reactive member. In some embodiments, the reaction time may be at least 30 min, at least 1 h, at least 1 h and 30 min, or at least 2 h. In some embodiments, the reaction time at least 2 h.

The inventors envisage that when the CMC derivative product comprising at least one unit of Formula 3 comprises a metal chelator (such as the biomolecular unit M is a metal chelator), the metal chelator chelates Zn²⁺ (or Zn(II)) (that is, it is capable of chelating Zn²⁺), which may promote wound healing by altering the activity of matrix metallopeptidases (MMPs), such as deactivating MMPs.

Matrix metallopeptidases (MMPs) are a family of zinc dependant endopeptidases that possess a catalytic domain in which a Zn(II) ion is coordinated to a tris (histidine) motif (Puerta et al., 2004). MMPs have been shown to display both beneficial and negative roles in wound healing. Their impact on wound healing depends on their level of accumulation over time at the site of the wound. Beneficial roles include the facilitation of cellular migration, extracellular matrix (ECM) homeostasis, angiogenesis and tissue remodelling (Menke et al., 2007). During the process of wound healing, MMP levels are found to vary, with levels significantly peaking during the inflammatory phase of wound healing, and then declining drastically as the wound resurfaces with new epithelial tissue (Menke et al., 2007).

Moreover, when comparing a healing wound to a chronic wound, levels of MMPs are found to be significantly higher in the latter. This difference is accounted for by chronic inflammation, during which a chronic wound is stuck in a continuous cycle of the inflammatory phase (Liu et al., 2009). As noted above, levels of MMPs are at their highest during the inflammatory phase, and such persistently high levels of MMPs have a detrimental effect to wound healing. Such effects include the degradation of newly deposited or existing ECM components such as collagen, glycosaminoglycans, proteoglycans, elastin and fibronectin, as well as the degradation of the various protein growth factors (PGFs) necessary for the facilitation of wound healing (Liu et al., 2009). This ECM and PGF degradation prevents the accumulation of granulation tissue, and as a result, the wound remains in a permanent chronic inflammatory state (Liu et al., 2009).

The inventors envisage that chelating Zn(II) to woundcare devices comprising the CMC derivatives described herein will decrease Zn(II) levels at the site of the wound and subsequently decrease the detrimental effect MMPs have on wound healing.

Furthermore, the inventors also envisage that metal chelators attached to CMC derivatives could be used to chelate and potentiate the cell walls of bacteria and destabilize bacterial biofilms by sequestering metal ions of calcium, magnesium, zinc, and iron. The metal ions may also have antibacterial properties, such as Ag(I). This would make the CMC derivatives described herein suitable as wound healing devices for the management of bacterial biofilms at the site of a wound.

Additionally, the inventors also envisage that metal chelators attached to CMC derivatives may chelate and sequester toxic metal ions, such as Pb(II), Pb(II), Ni(II) and Cr(III), to prevent the toxic metal ions entering the wound. The metal chelators may also complex metal ions, such as Fe(III), to catalyse the formation of reactive oxygen species (ROS) at the site of the wound, such as peroxide (O₂²⁻), hydrogen peroxide (H₂O₂) and super-oxide anions (O²⁻).

### Biomolecular Unit M is a Fluorescent Probe

The biomolecule of Formula B, such as Formula B1, may comprise a fluorescent probe as biomolecular unit M. The group R² may comprise a fluorescent probe (fluorophore) as biomolecular unit M.

A fluorescent probe or fluorophore is a functional group or moiety that is able to emit fluorescent light upon light excitation, such as upon the absorption of light. The functional group or moiety may comprise aromatic groups, such as aryl. An example of a known fluorescent probe or fluorophore is fluorescein.

When the biomolecular unit M is a fluorescent probe, the fluorescent probe may be Formula F:

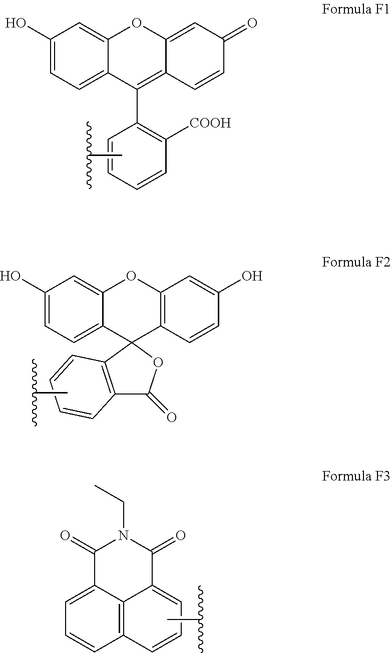

In some embodiments, the fluorescent probe for the biomolecular unit M of Formula F1 is:

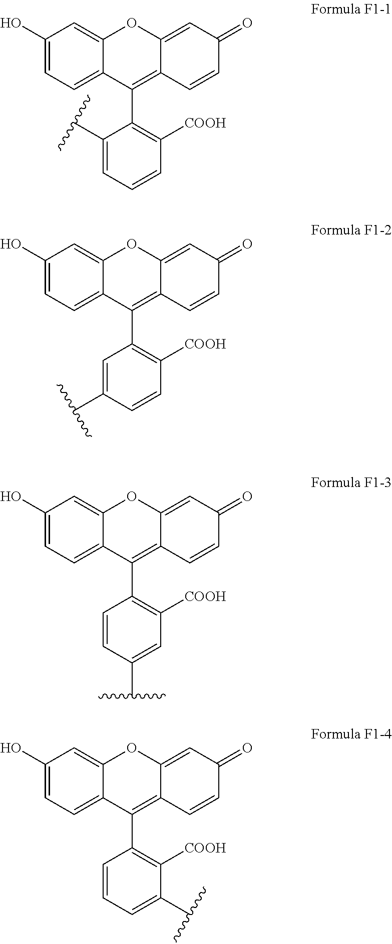 [SMILES: *c1cccc(C(=O)O)c1-c1c2ccc(=O)cc-2oc2cc(O)ccc12 ; *c1ccc(C(=O)O)c(-c2c3ccc(=O)cc-3oc3cc(O)ccc23)c1 ; *c1ccc(-c2c3ccc(=O)cc-3oc3cc(O)ccc23)c(C(=O)O)c1 ; *c1cccc(-c2c3ccc(=O)cc-3oc3cc(O)ccc23)c1C(=O)O]

In some embodiments, the fluorescent probe for the biomolecular unit M is Formula F1-1. In some embodiments, the fluorescent probe for the biomolecular unit M is Formula F1-2. In some embodiments, the fluorescent probe for the biomolecular unit M is Formula F1-3. In some embodiments, the fluorescent probe for the biomolecular unit M is Formula F1-4.

In some embodiments, the fluorescent probe for the biomolecular unit M may be a mixture of two or more of Formula F1-1, Formula F1-2, Formula F1-3, or Formula F1-4. In some embodiments, the fluorescent probe for the biomolecular unit M is a mixture of Formula F1-2 and Formula F1-3.

In some embodiments, the fluorescent probe for the biomolecular unit M of Formula F2 is:

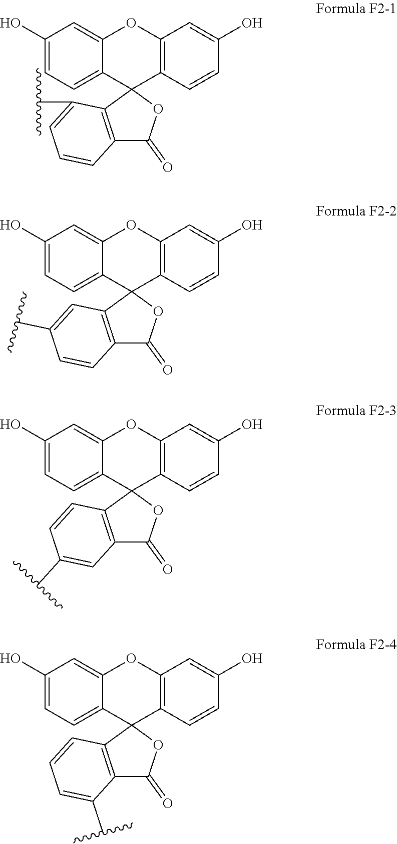 [SMILES: *c1cccc2c1C1(OC2=O)c2ccc(O)cc2Oc2cc(O)ccc21 ; *c1ccc2c(c1)C1(OC2=O)c2ccc(O)cc2Oc2cc(O)ccc21 ; *c1ccc2c(c1)C(=O)OC21c2ccc(O)cc2Oc2cc(O)ccc21 ; *c1cccc2c1C(=O)OC21c2ccc(O)cc2Oc2cc(O)ccc21]

In some embodiments, the fluorescent probe for the biomolecular unit M is Formula F2-1. In some embodiments, the fluorescent probe for the biomolecular unit M is Formula F2-2. In some embodiments, the fluorescent probe for the biomolecular unit M is Formula F2-3. In some embodiments, the fluorescent probe for the biomolecular unit M is Formula F2-4.

In some embodiments, the fluorescent probe for the biomolecular unit M of Formula F3 is:

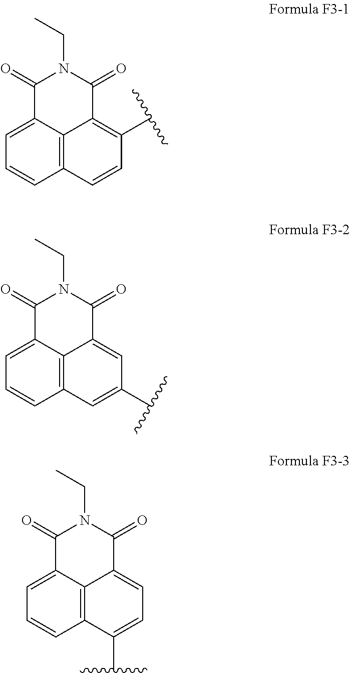 [SMILES: *c1ccc2cccc3c2c1C(=O)N(CC)C3=O ; *c1cc2c3c(cccc3c1)C(=O)N(CC)C2=O ; *c1ccc2c3c(cccc13)C(=O)N(CC)C2=O]

In some embodiments, the fluorescent probe for the biomolecular unit M is Formula F3-1. In some embodiments, the fluorescent probe for the biomolecular unit M is Formula F3-2. In some embodiments, the fluorescent probe for the biomolecular unit M is Formula F3-3.

When the biomolecular unit M is a fluorescent probe connected to the linker group L², the linker group L2 is preferably -(alkylene)NHC(O)—* or a bond, wherein the*is the attachment to M. In some embodiments, the linker group L² is preferably —(C₁-C₆alkylene)NHC(O)—*. In some embodiments, the linker group L² is —(C₃ alkylene)NHC(O)—*, such as -(n-propylene)NHC(O)—*. In some embodiments, the linker group L² is a bond.

When the biomolecular unit M is a fluorescent probe and the linker group L² is a bond, the group R² is the same as the biomolecular unit M of Formula F1, such as Formula F1-1, Formula F1-2, Formula F1-3, and Formula F1-4; or Formula F2, such as Formula F2-1, Formula F2-2, Formula F2-3, and Formula F2-4; or Formula F3, such as Formula F3-1, Formula F3-2, and Formula F3-3.

When the biomolecular unit M is a fluorescent probe, the group R² may be Formula F1-1a, Formula F1-2a, Formula F1-3a, or Formula F1-4a:

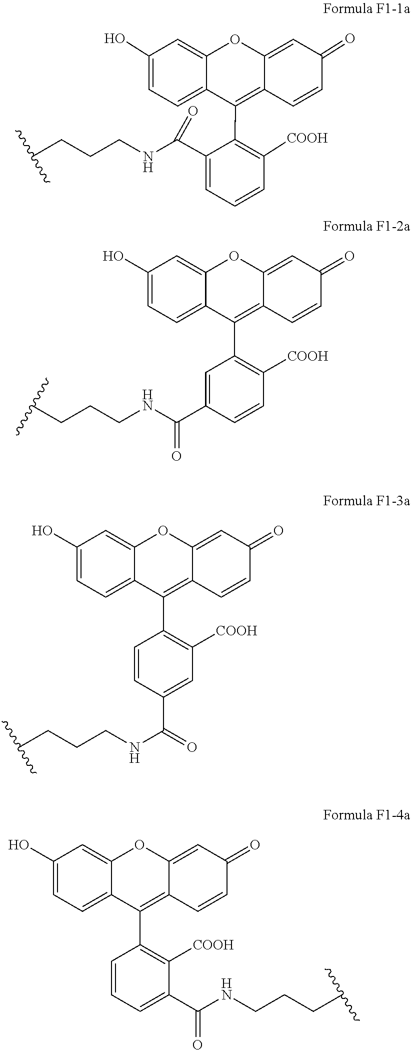 [SMILES: *CCCNC(=O)c1cccc(C(=O)O)c1-c1c2ccc(=O)cc-2oc2cc(O)ccc12 ; *CCCNC(=O)c1ccc(C(=O)O)c(-c2c3ccc(=O)cc-3oc3cc(O)ccc23)c1 ; *CCCNC(=O)c1ccc(-c2c3ccc(=O)cc-3oc3cc(O)ccc23)c(C(=O)O)c1 ; *CCCNC(=O)c1cccc(-c2c3ccc(=O)cc-3oc3cc(O)ccc23)c1C(=O)O]

In some embodiments, the group R² may be Formula F1-1a. In some embodiments, the group R² may be Formula F1-2a. In some embodiments, the group R² may be Formula F1-3a. In some embodiments, the group R² may be Formula F1-4a.

In some embodiments, the group R² may be a mixture of two or more of Formula F1-1a, Formula F1-2a, Formula F1-3a, or Formula F1-4a. In some embodiments, the group R² is a mixture of Formula F1-2a and Formula F1-3a.

When the biomolecular unit M is a fluorescent probe, the group R² may be Formula F2-1a, Formula F2-2a, Formula F2-3a, or Formula F2-4a:

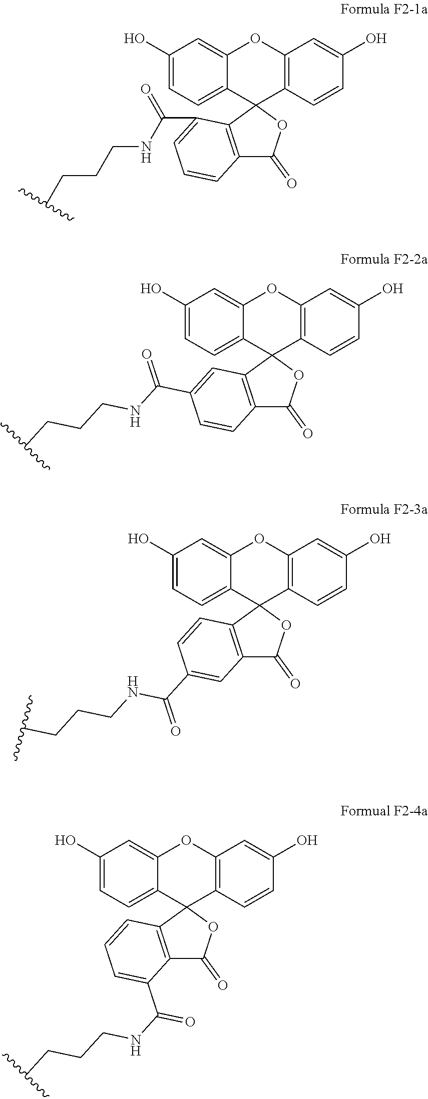 [SMILES: *CCCNC(=O)c1cccc2c1C1(OC2=O)c2ccc(O)cc2Oc2cc(O)ccc21 ; *CCCNC(=O)c1ccc2c(c1)C1(OC2=O)c2ccc(O)cc2Oc2cc(O)ccc21 ; *CCCNC(=O)c1ccc2c(c1)C(=O)OC21c2ccc(O)cc2Oc2cc(O)ccc21 ; *CCCNC(=O)c1cccc2c1C(=O)OC21c2ccc(O)cc2Oc2cc(O)ccc21]

In some embodiments, the group R² may be Formula F2-1a. In some embodiments, the group R² may be Formula F2-2a. In some embodiments, the group R² may be Formula F2-3a. In some embodiments, the group R² may be Formula F2-4a.

When the biomolecular unit M is a fluorescent probe, the group R² may be Formula F3-1a, Formula F3-2a, or Formula F3-3a:

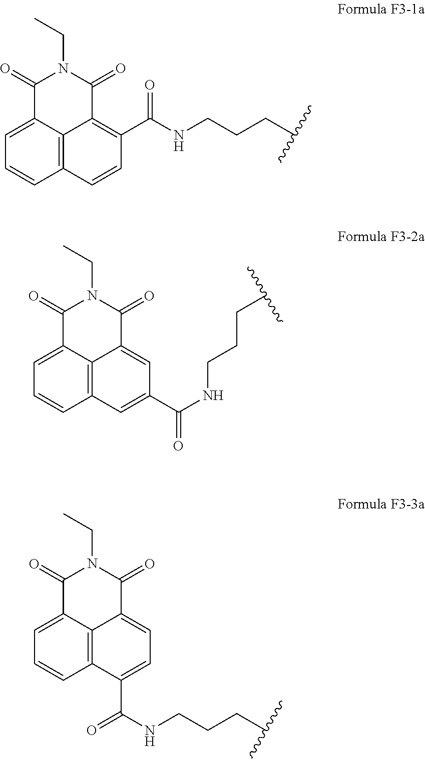 [SMILES: *CCCNC(=O)c1ccc2cccc3c2c1C(=O)N(CC)C3=O ; *CCCNC(=O)c1cc2c3c(cccc3c1)C(=O)N(CC)C2=O ; *CCCNC(=O)c1ccc2c3c(cccc13)C(=O)N(CC)C2=O]

In some embodiments, the group R² may be Formula F3-1a. In some embodiments, the group R² may be Formula F3-2a. In some embodiments, the group R² may be Formula F3-3a.

When the biomolecular unit M is a fluorescent probe in step iii), the reaction may be conducted at about room temperature.

When the biomolecular unit M is a fluorescent probe in step iii), the reaction solvent may be mixture of reaction solvents. The mixture of reaction solvents may be a polar (hydrophilic) aprotic solvent and a buffer solution.

The polar (hydrophilic) aprotic solvent may be tetrahydrofuran (THF), N,N′-dimethylformamide (DMF), hexamethylphosphoramide (HMPA), or dimethylsulfoxide (DMSO). In some embodiments, the polar (hydrophilic) aprotic solvent is dimethylsulfoxide (DMSO).

The buffer solution may be 2-(N-morpholino)ethanesulfonic acid (MES) buffer, 3-(N-morpholino) propanesulfonic acid (MOPS) buffer, 4-(2-hydroxyethyl)-1-piperazineethanesulfonic acid (HEPES) buffer, or phosphate-buffered saline (PBS). In some embodiments, the buffer solution is phosphate-buffered saline (PBS). The pH of the buffer solution may be from about pH 7 to about pH 8, such as about pH 7.4.

In some embodiments, the mixture of reaction solvents may be dimethylsulfoxide (DMSO) and phosphate-buffered saline (PBS). The ratio of the mixture of reaction solvents of polar (hydrophilic) aprotic solvent: buffer solution may be 1:1, 2:1, 3:1 or 4:1. In some embodiments, the mixture of reaction solvents is dimethylsulfoxide (DMSO): phosphate-buffered saline (PBS) in the ratio 1:1.

When the biomolecular unit M is a fluorescent probe, the reaction of step iii) is conducted until there is no further change in the composition of the reaction mixture, such as when all of the CMC derivative comprising at least one unit of Formula 2 has reacted with the biomolecule comprising the complementary reactive member. In some embodiments, the reaction time may be at least 10 h, at least 12 h, at least 14 h, at least 16 h, at least 18 h, at least 20 h, at least 22 h, or at least 24 h. In some embodiments, the reaction time is overnight, such as at least 16 h.

The inventors envisage that when the CMC derivative product comprising at least one unit of Formula 3 comprises a fluorescent probe (such as the biomolecular unit M is a fluorescent probe or fluorophore), the fluorescent probes of the CMC derivative product may be used as a diagnostic device in a functionalised wound dressing.

Fluorescent probes can give a clear signal from solid surfaces (like CMC) and are not prone to interference from biological fluids. In particular, fluorescein derivatives have pH-sensitive emission, particularly around physiologically relevant pH values (Le Guern et al., 2020; Bidmanova et al., 2012), so they could report on wound pH. Furthermore, 4-aminonaphthalimide (Lucifer) derivatives are non-toxic and have strong emission in water (Stewart, 1981).

### Biomolecular Unit M is a Peptide

The biomolecule of Formula B, such as Formula B1, may comprise a peptide as biomolecular unit M. The group R² may comprise a peptide as biomolecular unit M.

A peptide is a chain of amino acid residues linked by peptide (amide) bonds. The amino acid residues may be derived from proteinogenic or non-proteinogenic amino acids, such as α-aminoisobutyric acid (Aib).

Preferably, the N-terminus of the peptide chain is connected to the linker group L².

When the biomolecular unit M is a peptide, the peptide may be Formula P:

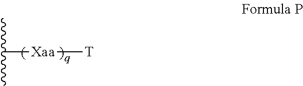

wherein Xaa is a natural or unnatural amino acid residue, q is the number of amino acid residues in the chain length, and T may be —H, —OH, —OR^{O2}, —NH₂, —NHR^{N2}, or NR^{N2}₂, wherein R^{O2} is alkyl optionally substituted with halogen, and each R^{N2} is alkyl optionally substituted with halogen.

When R^{O2} or R^{N2} is present and is substituted with halogen, the halogen is preferably —F.

The peptide chain of amino acid residues may be of any chain length q. In some embodiments, the peptide chain of amino acid residues q may be from 1 to 10 amino acid residues in length. In some embodiments, the peptide chain of amino acid residues q may be from 1 to 5 amino acid residues in length. In some embodiments, the peptide chain of amino acid residues q is 3 amino acid residues in length.

In some embodiments, the peptide M may be:

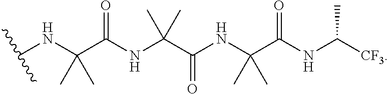 [SMILES: *NC(C)(C)C(=O)NC(C)(C)C(=O)NC(C)(C)C(=O)N[C@H](C)C(F)(F)F]

When the biomolecular unit M is peptide connected to the linker group L², the linker group L² is preferably -(alkylene)C(O)—*, wherein the*is the attachment to M. In some embodiments, the linker group L² is preferably —(C₁-C₆ alkylene)C(O)—*. In some embodiments, the linker group L² is —(C₃ alkylene)C(O)—*, such as —(C(CH₃)₂)C(O)—*.

When RCM is an azide functional group (—N₃) in Formula B such as in Formula B1, the linker group L2 may be derived from a natural or unnatural amino acid residue wherein the N-terminus is converted into the azide functional group (—N₃) and the C-terminus is the attachment to M.

When the biomolecular unit M is a peptide, the group R² may be:

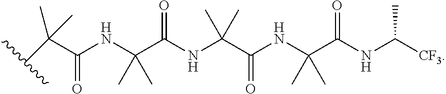 [SMILES: *C(C)(C)C(=O)NC(C)(C)C(=O)NC(C)(C)C(=O)NC(C)(C)C(=O)N[C@H](C)C(F)(F)F]

When the biomolecular unit M is a peptide in step iii), the reaction may be conducted at about room temperature.

When the biomolecular unit M is a peptide in step iii), the reaction solvent may be mixture of reaction solvents. The mixture of reaction solvents may be a polar (hydrophilic) aprotic solvent and water. The water may be distilled water.

The polar (hydrophilic) aprotic solvent may be tetrahydrofuran (THF), N,N′-dimethylformamide (DMF), hexamethylphosphoramide (HMPA), or dimethylsulfoxide (DMSO). In some embodiments, the polar (hydrophilic) aprotic solvent is N,N′-dimethylformamide (DMF).

In some embodiments, the mixture of reaction solvents may be N, N′-dimethylformamide (DMF) and water. The ratio of the mixture of reaction solvents of polar (hydrophilic) aprotic solvent:water may be 1:1, 2:1, 3:1 or 4:1. In some embodiments, the mixture of reaction solvents is N,N′-dimethylformamide (DMF):water in the ratio 1:1.

When the biomolecular unit M is a peptide, the reaction of step iii) is conducted until there is no further change in the composition of the reaction mixture, such as when all of the CMC derivative comprising at least one unit of Formula 2 has reacted with the biomolecule comprising the complementary reactive member. In some embodiments, the reaction time may be at least 10 h, at least 12 h, at least 14 h, at least 16 h, at least 18 h, at least 20 h, at least 22 h, or at least 24 h. In some embodiments, the reaction time is overnight, such as at least 16 h.

The inventors envisage that when the CMC derivative product comprising at least one unit of Formula 3 comprises a peptide (such as the biomolecular unit M is a peptide), the peptide of the CMC derivative product may be used as a signalling ligand that can alter cell behaviour, for example peptide-based analogues of native ECM components, such as RGD (tripeptide Arg-Gly-Asp), YIGSR (peptide Tyr-Ile-Gly-Ser-Arg), and IKVAV (peptide Ile-Lys-Val-Ala-Val), may promote cell adhesion and cell proliferation in or on these CMC derivatives (Kharkar et al., 2013). Alternatively, the peptides may have antimicrobial activity that suppresses wound infection, for example the peptaibols are a class of membrane active antimicrobial peptide that contains high proportions of Aib (Daniel et al., 2007).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

An alkyl group may be linear or branched, such as linear. An alkyl group may be a C₁₋₆ alkyl group. The C₁₋₆ alkyl group may be C₁₋₃ alkyl, such as C₁₋₂ alkyl, such as C₁ alkyl (methyl). The C₁₋₆ alkyl may be a C₁ alkyl such as methyl. The C₁₋₆ alkyl may be a C₂ alkyl such as ethyl. The C₁₋₆ alkyl may be a C₃ alkyl such as n-propyl or iso-propyl. The C₁₋₆ alkyl may be a C₄ alkyl such as n-butyl.

An aryl group may be carboaryl or heteroaryl, such as C₆₋₁₀ carboaryl or C₅₋₁₀ heteroaryl. A heteroaryl group may be attached via a carbon ring atom, or alternatively via a nitrogen ring atom, where such is present and available. A C₆₋₁₀ carboaryl group may be phenyl. A C₅₋₁₀ heteroaryl group may be C₅₋₆ heteroaryl. A C₅ heteroaryl may be a group selected from furanyl, pyrrolyl, thiophenyl, oxazolyl, isoxazolyl, thiazolyl, isothiazolyl, imidazolyl and pyrazolyl, such as isoxazolyl and pyrazolyl. A C₆ heteroaryl may be a group selected from pyridinyl, pyridazinyl, pyrimidinyl, pyrazinyl and triazinyl. A C₁₀ heteroaryl may be a group selected from quinolinyl, isoquinolinyl and umbelliferyl. The aryl group may be optionally substituted where one or more —H is replaced with a substituent, such as with an alkyl, halogen, —OH, —O (alkyl), —O(acyl), or a nitro group.

An acyl group is a —C(O)R group, wherein R is an alkyl group. The acyl group may be acetyl (Ac or —C(O)CH₃), wherein R is methyl (—CH₃).

An alkylene group is a bivalent saturated aliphatic radical derived from an alkene group by opening of the double bond or from an alkyl group by removal of two hydrogen atoms from different carbon atoms. An alkylene group may be linear or branched, such as linear. An alkylene group may be a C₁₋₆ alkylene group. The C₁₋₆ alkylene group may be C₁₋₃ alkylene, such as C₁₋₂ alkylene, such as C₁ alkylene (methylene). The C₁₋₆ alkylene may be a C₁ alkylene such as methylene. The C₁₋₆ alkylene may be a C₂ alkylene such as ethylene. The C₁₋₆ alkylene may be a C₃ alkylene, such as n-propylene. The C₁₋₆ alkylene may be a C₄ alkylene, such as n-butylene. The C₁₋₆ alkylene may be a branched C₁₋₆ alkylene, such as a branched C₃ alkylene, such as —C(CH₃)₂—.

An arylene group is a bivalent aryl radical derived from an aryl group by removal of two hydrogen atoms from different carbon atoms. An arylene group may be a C₆ arylene, such as phenylene. The radicals may be at any carbons, such as C-1, C-2, C-3, C-4, C-5, or C-6 if present. The radicals on a phenylene may be at C-1 and C-4 to minimise steric hindrance.

### EXAMPLES

### General Materials

All reagents and solvents were obtained from commercial suppliers; (Sigma-Aldrich, Fisher Scientific and Thermo Scientific), unless stated otherwise. Carboxymethylcellulose sodium salt was purchased from Sigma-Aldrich (C4888, Medium viscosity, Sodium (Na) 6.5-9.5%, Brookfield Viscosity 400-800 cps, 2% in H₂O at 25 deg C, Degree of Substitution 0.65-0.90 carboxymethyl group per anhydroglucose unit). The modified saccharide GlcNAc—CH₂CH₂CH₂—N₃ was a gift from Dr Aisling Ni Cheallaigh. The peptide N₃Aib₄NHCH(CF₃)CH₃ was a gift from Mr Siyuan Wang. Bath-sonication was performed by use of a Transsonic™ T460 bath-type sonicator. All pH measurements were recorded on a HANNA pH 212-microprocessor pH meter. Reactions at 4° C. and 0° C. were performed using an ice bath. Compounds were dried on a rotary evaporator connected to a high vacuum system to remove residual solvent.

### Instrumentation

Solution ¹H, ¹³C, ³¹P, ¹⁹F NMR spectra were recorded on 400 and 500 MHz Bruker DPX spectrometers. ¹H NMR shifts were referenced to the residual deuterated solvent peak (CDCl₃; 7.27 ppm), ¹³C NMR shifts were referenced to the carbon resonance of the solvent (CDCl₃; 77.0 ppm). ³¹P NMR shifts were referenced to 85% phosphoric acid (0.0 ppm). ¹⁹F NMR shifts were referenced to trifluoromethane (0.0 ppm).

Solid-state NMR spectra were recorded using a Bruker Advance III 400 NMR spectrometer. Carbon (¹³C, filter in the range of 70-125 MHz) and fluorine (¹⁹F, filter in the range of 70-125 MHz) were analysed using a 4 mm dual probe: Bruker HP WB73 MAS 4 BL CP BB DVT; and phosphorus (³¹P, filter in the range of 120-205 MHz) using the probe 4 mm triple probe: Bruker HP WB73 MAS 4 BL CP TRIPLE. The following external standards were used in each measurement: adamantane (¹³C standard), ammonium dihydrogen phosphate (ADP, ³¹P standard) and hexafluorobenzene (¹⁹F standard). NMR and ssNMR data were analysed using MestreNova. Resonances from the 1,4 and 1,5 triazole regioisomers were not resolved: only the 1,4-isomer is depicted in the text below.

Coupling constants (J) are reported in Hertz (Hz) with chemical shifts being recorded in parts-per-million (ppm). Multiplicities are reported with the appropriate abbreviations; singlet(s), doublet (d), triplet (t), doublet of doublet (dd), quintet (p), doublet of quartets (dq) and multiplet (m). Mestrelabs MestReNova software package was used to analyse all NMR spectra. Assignments of resonances were made using 2D ¹H-COSY and TOCSY where relevant.

Elemental analysis (EA) of carbon, hydrogen, and nitrogen were performed using a Thermo Scientific FLASH 2000 series CHNS/O analyser. Phosphorus elemental analysis was performed by ICP-OES (Inductively Coupled Plasma Optical Emission Spectrometry) on a Thermo Scientific iCAP 6300 Duo system. Electrospray mass spectrometry was performed on a Micromass LCT using a Waters 2790 separation module with electrospray ionisation and TOF fragment detection. Fourier transform infrared (FTIR) spectra were obtained by use of a Bruker Alpha-P instrument with OPUS 6.5 software package. Raman spectroscopy was performed on an InVia™ Raman spectrometer from Renishaw with OPUS 6.5 software package, using a laser of wavelength 532 nm and power of 100% or 50%.

### Kaiser Assay Procedure

Reagent A: Dissolve 16.5 mg of KCN in 25 mL of distilled water. Dilute 1.0 mL of above solution with 49 mL of pyridine (freshly distilled from ninhydrin).

Reagent B: Dissolve 1.0 g of ninhydrin in 20 mL of n-butanol.

The relevant CMC derivative (5 mg) was subjected to reagent A (100 μL) and reagent B (25 μL) with agitation at 90° C. for 10 minutes and covered with foil to protect from light.

The sample was immediately placed in a cold water bath of 4° C. Cold ethanol (15 mL) was added to each test solution and mixed well before being analysed by UV-visible spectroscopy. An aliquot (2 mL) was taken for each sample and the absorbance measured at 570 nm.

The absorbance was interpolated from a standard curve to obtain the concentration of glycine amine groups in each sample. The standard curve consisted of known concentrations of glycine ranging from 0.01 to 0.48 mg/mL in 2 mL. Each solution was subjected to reagent A (100 μL) and reagent B (25 μL) with agitation at 90° C. for 10 minutes and covered with foil to protect from light. The standard curve sample sample was immediately placed in a cold water bath of 4° C. Cold ethanol (15 mL) was added to each test solution and mixed well before being analysed by UV-visible spectroscopy. An aliquot (2 mL) was taken for each sample and the absorbance measured at 570 nm.

For example the absorbance for CMC-PEG₍ₙ₌₃₎-NH₂ (Example 1) was 0.92314 and the interpolated concentration was 0.39 mmol/L in the 2 mL volume, which comprises all the available amine in the 5 mg CMC sample.

### Examples 1 and 2—Synthesis of CMC-Spacer Compounds

The first step is to couple Na-CMC to a spacer. To achieve this, Na-CMC can be coupled to diamine PEG₍ₙ₎-NH₂ via amide coupling to form CMC-PEG₍ₙ₎-NH₂ (Scheme 4).

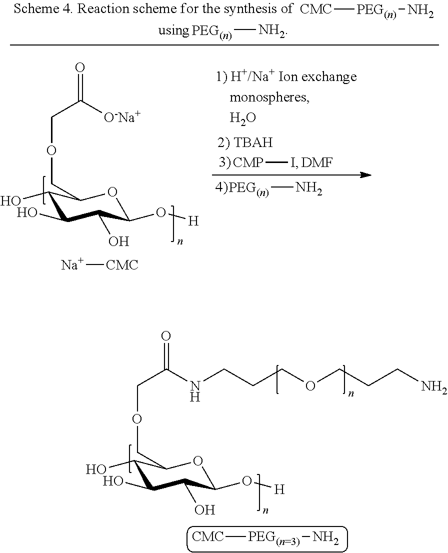

The percentage conversion is the number of spacer groups attached compared to the total available carboxylate groups on the CMC polymer. The percentage conversion from Na-CMC to the CMC-spacer compound can be calculated using the elemental analysis of nitrogen in the sample. The percentage conversion calculations were performed by first calculating the maximum possible percentage of nitrogen groups in the product bearing in mind the original degree of substitution of the CMC raw material and ignoring any residual tetrabutylammonium counterion that may have been carried through from earlier reaction steps. The actual percentage of nitrogen given by elemental analysis was then divided by the maximum calculated percentage to give a percentage conversion or DoC. The percentage conversion is then multiplied by the DoS of the Na-CMC to give the DoS of the CMC-spacer compound.

The ninhydrin assay (Kaiser Test) is a very sensitive test to quantitate primary amines and was also used to calculate the DoS of CMC-spacer compound. Ninhydrin reacts with the primary amine of CMC-spacer compound to form an intense blue complex which can be detected using UV-spectroscopy. Using the absorbance of the intense blue complex at 570 nm, primary amines from the spacer can be quantitated to calculate the DoS.

### Example 1—Synthesis of CMC-PEG₍ₙ₌₃₎-NH₂

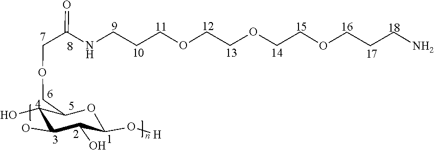

Na-CMC powder (DoS: 0.65-0.90) (2.00 g, 8.33 mmol) was dissolved in water (180 mL) to give a 1% solution. Dowex 650C monosphere ion exchange resins were added and the resulting solution was stirred for approximately 15 minutes. The monospheres were removed by gravity filtration before a 40% (aq) tetrabutylammonium hydroxide solution was added in 0.3 mL aliquots, until the pH of the solution reached 8-9 (3.0 mL, 1.16 mmol). The resulting solution was stirred for 30 minutes before being lyophilized. The lyophilized material was dissolved in dry dimethylformamide (250 mL) under nitrogen atmosphere, by stirring and gently heating to 40° C. over a period of approximately 6 hours. The solution was cooled to approximately 4° C., and 2-chloro-1-methylpyridinium iodide (1.48 g, 5.8 mmol) was added with vigorous stirring for 6 hours. 4,7,10-Trioxa-1,13-tridecandiamine (12.020 g/2.020 mL, 9.17 mmol) was then added to the reaction solution, along with dry triethylamine (5 mL). The reaction solution was kept at 4° C. and stirred for a minimum of 3 hours, then cold (0° C.) 99% acetone (100 mL) was slowly added while stirring to precipitate a white product. The product was filtered, washed with acetone (3×100 mL), ethanol (3×100 mL), water (1×100 mL), hexane (1×100 mL), and then again with water (1×100 mL). The product was concentrated in vacuo and lyophilized overnight (16h) to afford the titled compound as a white compressible solid (1.47 g, 40% mass recovery). Solid State ¹³C NMR: (10,000 MHz CP MAS) 176.21 (C-8 of Na-CMC), 171.37 (C-8), 130.52, 102.97 (C-1), 97.03, 81.25 (C-4), 74.14 (C-2, C-3, C-5), 70.39 (C-7), 61.39 (C-6), 36.53-28.19 (C-9, C-10, C-11, C-12, C-13, C-14, C-15, C-16, C-17, C-18). FTIR (vₘₐₓ/cm⁻¹) 3292 (O—H), 2871 (N—H, C—H), 1650 (CONH, amide of coupled product), 1589 (HNCOO, CMC), 1388 (COO), 1023 (CN, primary amine). EA calculated C: 49.08%, H: 8.24%, N: 6.36%, found C: 45.97%, H: 6.93%, N: 3.74%.

DoS by elemental analysis: 3.74% N wt/wt, corresponding to 0.0374 g/g. MW N=14.01 g/mol, so 0.00267 mol/g. Only ½ is amine (1 eq. amine, 1 eq. amide), so up to 1.3 mmol/g, DoC=1.3/3.21=0.40, therefore DoS=(1.3/3.21)×0.7=0.28.

DoS by Kaiser test: This assay gave 1.18 mmol/g available amine groups, so total primary amine is >1.18 mmol/g (available amine from Kaiser test) and <1.34 mmol/g (expected total amine from elemental analysis). The average DoS of the reacting Na-CMC is 0.7, corresponding to 3.2 mmol/g of carboxylate, so the DoC of carboxylate into reactive primary amine (available to the Kaiser test) is 0.37. The average Dos per D-anhydroglucopyranose monomers of cellulose for this step is 0.26 according to this measure.

### Example 2—Synthesis of CMC-PEG₍ₙ₌₂₎-NH₂

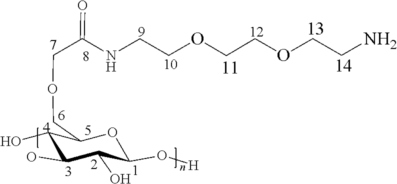

Na-CMC powder (DoS: 0.65-0.90) (204 mg, 0.85 mmol) was dissolved in water (20 mL) to give a 1% solution. Dowex 650C monosphere ion exchange resins were added and the resulting solution was stirred for approximately 15 minutes. The monospheres were removed by gravity filtration before a 40% (aq) tetrabutylammonium hydroxide solution was added in 50 uL aliquots, until the pH of the solution reached 8-9 (300 μL, 0.12 mmol). The resulting solution was stirred for 30 minutes before being lyophilized. The lyophilized material was dissolved in dry dimethylformamide (25 mL) under nitrogen atmosphere, by stirring and gently heating to 40° C. over a period of approximately 2 hours. The solution was cooled to approximately 4° C., and 2-chloro-1-methylpyridinium iodide (151 mg, 0.59 mmol) was added with vigorous stirring for 4 hours. 2,2′-(ethylenedioxy)bis(ethylamine) (135 μL, 0.93 mmol) was then added to the reaction solution, along with dry triethylamine (0.5 mL). The reaction solution was kept at 4° C. and stirred for a minimum of 3 hours, before cold (0° C.) 99% acetone (25 mL) was slowly added while stirring to precipitate a white product. The product was filtered, washed with acetone (3×20 mL), ethanol (3×20 mL), H₂O (1×20 mL), hexane (1×20 mL), and then again with water (1×20 mL). The product was concentrated in vacuo and lyophilized overnight (16 h) to afford the titled compound as an off-white brittle solid (136 mg, 43% mass recovery). Solid State ¹³C NMR: (10,000 MHz CP MAS) 176.94 (C-8 of Na⁺-CMC), 171.60 (C-8), 132.24, 103.18 (C-1), 97.38, 82.00 (C-4), 74.17 (C-2, C-3, C-5), 70.39 (C-7), 61.75 (C-6), 39.44-24.05 (C-9, C-10, C-11, C-12, C-13, C-14). FTIR (vₘₐₓ/cm⁻¹): 3280 (O—H), 2861 (N—H, C—H), 1653 (CONH, amide of coupled product), 1580 (HNCOO, CMC), 1383 (COO), 1028 (CN, primary amine). EA calculated C: 45.65%, H: 7.66%, N: 7.60%, found C: 45.96%, H: 6.74%, N: 5.32%. DoS by elemental analysis: 5.32% N wt/wt, corresponding to 0.0532 g/g. MW N=14.01 g/mol, so 0.00380 mol/g. Only ½ is amine (1 eq. amine, 1 eq. amide), so up to 1.9 mmol/g, DoC 1.9/3.21=0.59, therefore DoS=(1.9/3.21)×0.7=0.41.

### Examples 3 to 5—Synthesis of CMC-Reactive Member Compounds

The second step is to couple the CMC-spacer with a reactive member in the form of a cyclooctyne derivative or Staudinger ligand possessing carboxylic acid functionality. Subsequently, CMC-PEG₍ₙ₎-NH₂ is coupled to the Staudinger ligand derivative 3-(diphenylphosphino)-4-(methoxycarbonyl)benzoic acid and cyclooctyne derivatives 2-(cyclooct-2-yn-1-yloxy)acetic acid and 1-fluorocyclooct-2-yne-1-carboxylic acid (Scheme 5). These reactions produce CMC-Staudinger, CMC-Cyclooctyne and CMC-F-Cyclooctyne respectively as CMC-reactive member compounds.

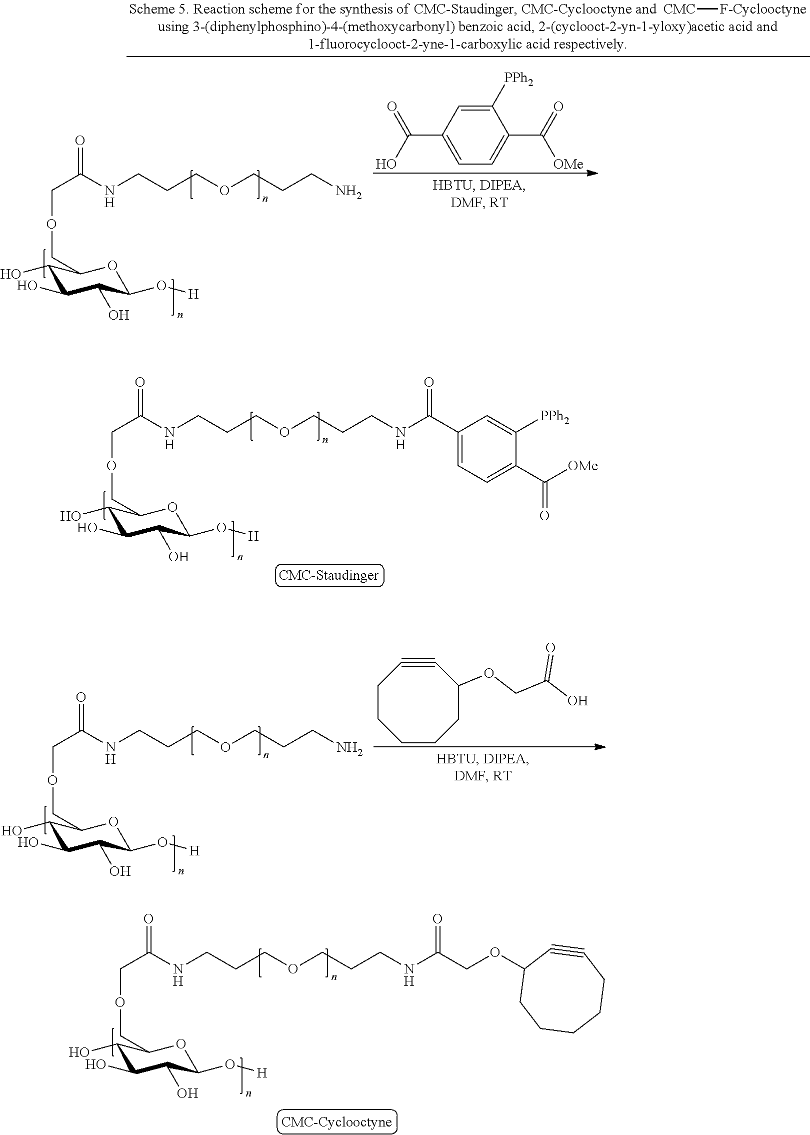

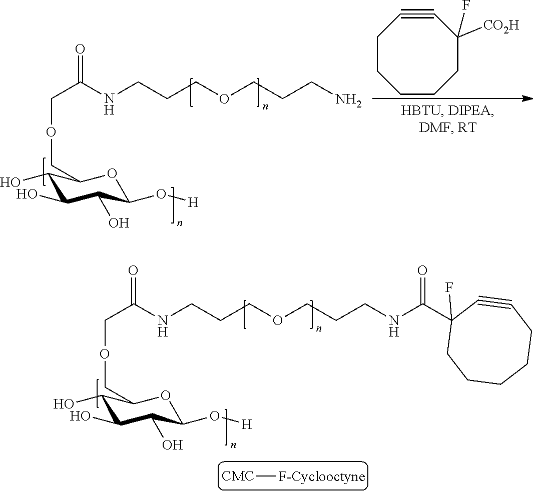

The DoS for CMC-Staudinger, CMC-Cyclooctyne and CMC-F-Cyclooctyne can be quantified using the ninhydrin assay (Kaiser Test). Elemental analysis can be used as an additional way to quantify DoS, but only for CMC-Staudinger. Elemental analysis may detect the presence of phosphorus in the sample for DoS quantification of CMC-Staudinger. Unfortunately, the available elemental analysis equipment was not able to quantify fluorine in the samples, so the DoS of CMC-F-Cyclooctyne cannot be reported this way.

### Example 3—Synthesis of CMC-F-Cyclooctyne

### Methyl (Z)-2-hydroxycyclooct-1-ene-1-carboxylate: methyl 2-oxocyclooctane-1-carboxylate (66:33)

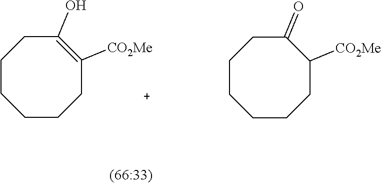 [SMILES: COC(=O)/C1=C(\O)CCCCCC1 ; COC(=O)C1CCCCCCC1=O]

After purging the apparatus with argon, a mixture of methanol (2.63 mL, 64.8 mmol) in dimethyl sulfoxide (3 mL) was added dropwise to a stirred suspension of oil-washed sodium hydride (60%, 6.48 g, 162 mmol) in dimethyl sulfoxide (140 mL) at room temperature. A solution of cyclooctanone (8.18g, 64.8 mmol) in dimethyl carbonate (21.8 mL, 259 mmol) was then added to the reaction mixture over 20 minutes. Excess sodium hydride was destroyed by addition of methanol (5.25 mL, 0.13 mmol). The reaction mixture was poured onto a solution of tartaric acid (12.2 g, 81 mmol), sodium chloride (10 g) and ice (100 g) dissolved in water (300 mL), and extracted with a mixture of petroleum ether: ethyl acetate (7:3, 3×100 mL). The organic layer was extracted, dried (MgSO₄), filtered and concentrated under reduced pressure to afford the titled compound in a tautomeric mixture (33:66) as yellow oil (12.80 g, 93%). Major tautomer denoted with*. ¹H NMR (400 MHz, CDCl₃, 25° C.) δ_{H}: 3.68 (3H, s, OCH₃)*, 3.62 (3H, s, OCH₃), 2.30 (4H, m, C (CH₂) 2), 1.39 (8H, m, (CH₂)₄). ¹³C NMR (400 MHz, CDCl₃, 25° C.) δ_{C}: 212.3 (CH₂CO), 176.5 (CCOH)*, 173.5 (COCH₃)*, 171.0 (COCH₃), 98.9 (CCOH)*, 56.7 (CHCOCH₃), 52.2 (COCH₃), 51.3 (COCH3)*, 41.9 (CH₂CHCO), 32.2 (CH₂CCO)*, 29.9 (CH₂CH₂CCO)*, 29.0 (CH₂CH₂CHCO), 28.6 (CH₂CH₂CH₂CCO)*, 27.0 (CH₂CH₂CH₂CHCCO), 26.5 (CH₂CH₂CH2COH)*, 26.0 (CH₂CH₂COH)*, 25.3 (CH₂CH₂CH₂CO), 25.2 (CH₂CH₂CO), 24.5 (CH₂CO), 23.9 (CH₂CO)*. MS (ES⁺, DCM) m/z: 207.0 [M-Na]⁺. HRMS calcd. for C₁₀H₁₆O₃ [M-Na]⁺ 207.0992, found 207.0982.

### Methyl 1-fluoro-2-oxocyclooctane-1-carboxylate

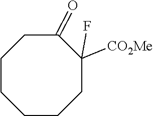 [SMILES: COC(=O)C1(F)CCCCCCC1=O]

To a solution of methyl (Z)-2-hydroxycyclooct-1-ene-1-carboxylate: methyl 2-oxocyclooctane-1-carboxylate (66:33) (4.5 g, 25 mmol) in anhydrous acetonitrile (80 mL), was added Selectfluor™ (10.4 g, 29 mmol) at 0° C. Under an argon atmosphere, the resulting mixture was stirred at 55° C. with use of an oil bath for 16 h. The reaction mixture was quenched by addition of water (200 mL) and extracted with ethyl acetate (3×200 mL). The combined organic layers were washed with brine (3×100 mL), dried (MgSO₄), filtered and concentrated under reduced pressure to afford a pale-yellow oil. The titled compound was afforded as a white solid (4.2 g, 21 mmol, 86%) by flash column chromatography (silica gel, 10% diethyl ether: petroleum ether, (R_{f}=0.14, 10% diethyl ether/petroleum ether)). ¹H NMR (400 MHz, CDCl₃, 25° C.) δ_{H}: 3.74 (s, 3H, COCH₃), 2.57 (m, 3H, CH₂COCFCHₐH_{b}), 2.19 (m, 1H, CH₂COCFCHₐH_{b}), 1.88 (m, 2H, CH₂CH₂COCF), 1.64 (m, 3H, CHₐH_{b}CH₂CH₂CH₂CO), 1.39 (m, 3H, CHₐH_{b}CH₂CH₂CF). ¹³C NMR (100 MHz, CDCl₃ 25° C.) δ_{C}: 208.7 (d, ²J_{C-F}=22.1 Hz, CO), 167.4 (d, ²J_{C-F}=24.8 Hz, COCH₃), 99.2 (d, ²J_{C-F}=199.5 Hz, CF), 53.3 (CH₃), 38.9 (CH₂CO), 33.5 (d, ²J_{C-F}=22.0 Hz, CH₂CF), 27.4 (d, ²J_{C-F}=2.5 Hz, CH₂CH₂CH₂CF), 26.5 (CH₂CH₂CO), 24.4 (CH₂CH₂CH₂CO), 21.3 (d, ²J_{C-F}=2.9 Hz, CH₂CH₂CF). ¹⁹F NMR (376 MHz, CDCl₃. 25° C.) δ_{F}: −171.6 (s). MS (ES⁺, DCM) m/z: 225.0 [M-Na]⁺. HRMS calcd. for C₉H₁₁FO₂ [M]⁺ 203.1078, found 203.1074.

### Methyl 1-fluorocyclooct-2-yne-1-carboxylate

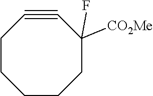 [SMILES: COC(=O)C1(F)C#CCCCCC1]

To a stirred solution of methyl 1-fluoro-2-oxocyclooctane-1-carboxylate (202 mg, 1 mmol) and N-phenyl-bis (trifluoromethanesulfonimide) (357 mg, 1 mmol) in anhydrous tetrahydrofuran (3 mL) was added potassium bis (trimethylsilyl) amide solution (1.1 mL, 1 M tetrahydrofuran, 1.1 mmol) at −78° C. The resulting mixture was stirred at −78° C. for 30 mins before it was quenched with saturated aqueous ammonium chloride (10 mL) and extracted with ethyl acetate (3×10 mL). The combined organic phases were washed with brine (10 mL), dried (MgSO₄), filtered and concentrated under reduced pressure to afford a dark brown oil. The titled compound was afforded as a clear oil (30 mg, 16%) by flash column chromatography (silica gel, 1-15% ethyl acetate: hexanes, R_{f}=0.25, 5% ethyl acetate: hexanes). ¹H NMR (400 MHz, CDCl₃, 25° C.) δ_{H}: 3.77 (s, 3H, COCH₃), 2.37-2.15 (m, 4H, (CH₂)₂(CH₂)₂CHₐH_{b}), 2.02-1.77 (m, 4H, (CH₂)₂(CH₂)₂CHₐH_{b}), 1.69-1.59 (m, 1H, (CH₂)₂CHₐH_{b}), 1.43-1.33 (m, 1H, (CH₂)₂CHₐH_{b}). ¹³C NMR (100 MHz, CDCl₃, 25° C.) δ_{C}: 169.2 (d, ²J_{C-F}=29.9 Hz, COCH₃), 108.7 (d, ²J_{C-F}=10.1 Hz, CH₂CCCF), 91.8 (d, ²J_{C-F}=184.8 Hz, CF), 87.0 (d, ²J_{C-F}=36.1 Hz, CH₂CCCF), 53.4 (COCH₃), 46.2 (d, ²J_{C—F}=24.8 Hz, CH₂CF), 33.9 (d, ²J_{C-F}=0.6 Hz, CH₂ (CH₂)₂), 29.3 (CH₂CH₂CC), 25.8 (d, ²J_{C-F}=0.6 Hz, CH₂CH₂CC), 20.8 (d, ²J_{C-F}=2.6 Hz, CH₂CH₂CF). 19F (376 MHz, CDCl₃, 25° C.) δ_{F}: −146.5 (s). MS (ES⁺, DCM) m/z: 207.1 [M-Na]⁺. HRMS calcd. for C₁₀H₁₃FO₂ [M-Na]⁺ 207.0797, found 207.0790.

### 1—Fluorocyclooct-2-yne-1-carboxylic acid

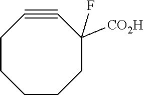 [SMILES: O=C(O)C1(F)C#CCCCCC1]

Methyl 1-fluorocyclooct-2-yne-1-carboxylate (25 mg, 0.13 mmol) and lithium hydroxide (6 mg, 0.26 mmol) were combined in 1 mL of 50% aqueous methanol. The mixture was heated to 50° C. by use of an oil bath for 15 mins. On completion, the reaction was cooled to room temperature and stirred for an additional 2 h. The reaction mixture was cooled to 0° C., diluted with water, and acidified to pH˜2 with 1 M aqueous hydrochloric acid. The mixture was extracted with ethyl acetate (3×50 mL) and the combined organic layers were dried (MgSO₄), filtered and then concentrated under reduced pressure to afford the titled compound as a yellow oil (18 mg 74%). ¹H NMR (400 MHz, CDCl₃, 25° C.) δ_{H}: 9.32 (bs, 1H, COOH), 2.45-2.20 (m, 4H, (CH₂)₂(CH₂) 2CHₐH_{b}), 2.06-1.75 (m, 4H, (CH₂)₂(CH₂) 2CHₐH_{b}), 1.74-1.60 (m, 1H, (CH₂)₂(CH₂)₂CHₐH_{b}), 1.48-1.32 (m, 1H, (CH₂)₂ (CH₂)₂CHₐH_{b}). ¹³C NMR (100 MHz, CDCl₃, 25° C.) δ_{C}: 173.7 (d, ²J_{C-F}=29.1 Hz, CO₂H), 109.4 (d, ²J_{C-F}=10.1 Hz, CH₂CCCF), 91.5 (d, ²J_{C-F}=185.8 Hz, CF), 86.1 (d, ²J_{C-F}=31.7 Hz, CH₂CCCF). 46.3 (d, ²J_{C-F}=24.7 Hz, CH₂CF), 33.8 (d, ²J_{C-F}=0.6 Hz, CH₂(CH₂)₂), 29.0 (CH₂CH₂CC), 25.4 (d, ²J_{C-F}=0.6 Hz, CH₂CH₂CC), 20.6 (d, ²J_{C-F}=2.6 Hz, CH₂CH₂CF). ¹⁹F (376 MHz, CDCl₃, 25° C.) δ_{F}: −147.0 (s). MS (ES⁻, DCM) m/z: 169.9 [M-H]⁻. HRMS calcd. for C₉H₁₁FO₂ [M-H]⁻ 169.0670, found 169.0669.

### CMC-F-Cyclooctyne (route 1)

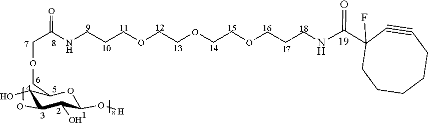

CMC-PEG₍ₙ₌₃₎-NH₂ (DoS: 0.35-0.58) (33 mg, 0.09 mmol) was suspended in anhydrous dimethylformamide (1 mL) under a nitrogen atmosphere. A solution of 1-fluorocyclooct-2-yne-1-carboxylic acid (18 mg, 0.11 mmol), O-(benzotriazol-1-yl)-N,N,N′,N′-tetramethyluronium hexafluorophosphate (HBTU, 42 mg, 0.11 mmol) and N,N-diisopropylethylamine (19 μL, 0.11 mmol) in anhydrous dimethylformamide (1.5 mL) was added to the suspension. The reaction mixture was stirred at room temperature overnight (16 h), before the product was filtered and washed with ethyl acetate (3×10 mL), acetone (3×10 mL) and water (10 mL) to afford the titled compound as a pale yellow compressible solid (34 mg, 64% mass recovery). Solid State ¹³C NMR (10,000 MHz CP MAS) 67 _{C}: 176.21 (C-8 of Na-CMC), 171.37 (C-8, C-19), 130.52, 102.97 (C-1), 97.03, 81.25 (C-4), 74.14 (C-2, C-3, C-5), 70.39 (C-7), 61.39 (C-6), 36.53-28.19 (C-9, C-10, C-11, C-12, C-13, C-14, C-15, C-16, C-17, C-18). Solid State ¹⁹F NMR (12,000 MHz, CP MAS) δ_{F}: −179.7 (C —F). FTIR (vₘₐₓ/cm⁻¹) 3292 (O-H), 2871 (N—H, C—H), 1650 (CONH, amide of coupled product), 1589 (HNCOO, CMC), 1388 (COO), 1315 (ar. C═C bending), 1023 (CN, primary amine). EA calculated C:54.72%, H:7.63%, N:4.73%, F:3.21% found C:45.64%, H:6.82%, N:3.44%, F: N/A. Raman (vₘₐₓ/cm⁻¹) 2329 (CC, alkyne), 1780-1650 (C═O, CONH of amide coupled product and HNCOO of CMC), 1640-1540 (C—C, of CMC backbone).

### CMC —F-Cyclooctyne (route 2 via Sulfo-NHS activation)

N-Hydroxysulfosuccinimide (510 mg, 2.35 mmol), 1-fluorocyclooct-2-yne-1-carboxylic acid (200 mg, 1.18 mmol) and 1-(3-dimethylaminopropyl)-3-ethylcarbodiimide hydrochloride (452 mg, 2.36 mmol) were dissolved in anhydrous dimethylformamide (8 mL). The resulting solution was stirred at room temperature for 2 hours. On completion, the reaction mixture was concentrated under reduced pressure to afford a bright red oil which was immediately added to a 1% w/v suspension of CMC-PEG-NH2 (DOS: 0.35-0.58) (520 mg, 1.18 mmol) in 520 mL of PBS buffer (pH 7.4). The resulting suspension was stirred at room temperature overnight (16 h) before the reaction mixture was lyophilized over several days to afford a dark orange solid. The resulting solid was washed with dimethylformamide (50 mL), dichloromethane (100 mL) and acetone (100 mL) to afford the titled compound as an orange compressible solid (535 mg, 64% mass recovery). Solid State ¹³C NMR (10,000 MHz CP MAS) δ_{C}: 176.21 (C-8 of Na-CMC), 171.37 (C-8, C-19), 130.52, 102.97 (C-1), 97.03, 81.25 (C-4), 74.14 (C-2, C-3, C-5), 70.39 (C-7), 61.39 (C-6), 36.53-28.19 (C-9, C-10, C-11, C-12, C-13, C-14, C-15, C-16, C-17, C-18). Solid State ¹⁹F NMR (12,000 MHz, CP MAS) δ_{F}: −179.7 (C —F). FTIR (vₘₐₓ/cm⁻¹) 3292 (O—H), 2871 (N—H, C—H), 1650 (CONH, amide of coupled product), 1589 (HNCOO, CMC), 1388 (COO), 1315 (ar. C═C bending), 1023 (CN, primary amine). EA calculated C:54.72%, H:7.63%, N:4.73%, F:3.21% found C:45.64%, H:6.82%, N:3.44%, F: N/A. Raman (vₘₐₓ/cm⁻¹) 2329 (CC, alkyne), 1780-1650 (C═O, CONH of amide coupled product and HNCOO of CMC), 1640-1540 (C—C, of CMC backbone).

DoS by Kaiser test: This assay gave 0.40 mmol/g available amine groups, so the reduction in available amine is 0.78 mmol/g; assume the conversion to cyclooctyne is 0.78 mmol/g. The amount of free amine on the CMC-PEG(n=3)-NH₂ is 1.18 mmol/g, so the DoC of reactive primary amine (available to the Kaiser test) into cyclooctyne is 0.66. The average DoS per D-anhydroglucopyranose monomers of cellulose for this step is 0.17 according to this measure.

### Example 4—Synthesis of CMC-Cyclooctyne

### Methyl (Z)-2-((2-bromocyclooct-2-en-1-yl)oxy)acetate

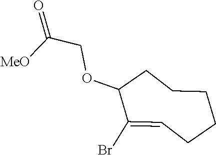 [SMILES: COC(=O)COC1CCCCC/C=C/1Br]

To a flame-dried aluminium foil-wrapped flask silver perchlorate (557 mg, 2.69 mmol) was added to a solution of 8,8-dibromobicyclo [5.1.0]octane (250 mg, 0.93 mmol) and methyl glycolate (781 μL, 10.3 mmol) in anhydrous toluene (4.5 mL). The reaction mixture was stirred for 3 h and then filtered through Celite to remove insoluble silver salts. The filtrate was concentrated under reduced pressure to afford a black oil. The titled compound was afforded as a light yellow oil (156 mg, 60%) by column chromatography (silica gel, 4-8% ethyl acetate: petroleum ether (R_{f}=0.33 in 8% ethyl acetate: petroleum ether)). ¹H NMR (400 MHz, CDCl₃, 25° C.) δ_{H}: 6.16 (dd, 1 H, J=11.7, 4.2 Hz), 4.18 (d, 1 H, J=16.5 Hz), 4.06 (dd, 1 H, J=10.3, 5.1 Hz), 3.92 (d, 1 H, J=16.5 Hz), 3.69 (s, 3 H), 2.68 (dq, 1 H), 2.23 (m, 1 H), 2.07-1.82 (m, 4 H), 1.66 (m, 1 H), 1.42 (m, 1 H), 1.24 (m, 1 H), 0.74 (m, 1 H). ¹³C NMR (100 MHz, CDCl₃) δ_{C}: 171.3, 133.3, 131.7, 85.1, 65.8, 52.1, 39.6, 36.8, 33.7, 28.3, 26.6. MS (ES⁺, DCM) 283.0 [M+Li]⁺. HRMS calcd. for C₁₁H₁₇O₃Br [M+Li]⁺ 283.0521, found 283.0515.

### (rac)-2-(Cyclooct-2-yn-1-yloxy)acetic acid

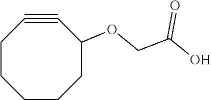 [SMILES: O=C(O)COC1C#CCCCCC1]

To a suspension of sodium methoxide (801 mg, 2.20 mmol) in anhydrous dimethyl sulfoxide (4 mL) was added a solution of methyl (Z)-2-((2-bromocyclooct-2-en-1-yl) oxy) acetate in anhydrous dimethyl sulfoxide (25 mL). The resulting solution was stirred at room temperature for 15 minutes before an additional solution of sodium methoxide (400 mg, 7.41 mmol in 2 mL of dimethyl sulfoxide) was added. The reaction was then stirred at room temperature until the starting material was completely consumed, as determined by thin-layer chromatography (˜45 minutes). On completion, water (1 mL) was added to the reaction solution before being allowed to stir for an additional hour. The reaction was then acidified by addition of 1 M HCl (300 mL) before being extracted with ethyl acetate (2×120 mL). The combined organic layers were then dried (MgSO₄), filtered and then concentrated under reduced pressure to afford a light yellow oil. The titled compound was afforded as a white waxy solid (650 mg, 73%) by silica gel chromatography (10:90:1-25:75:1 ethyl acetate: petroleum ether: acetic acid (R_{f}(25:75:1)=0.67)). ¹H NMR (400 MHz, CDCl₃, 25° C.) δ_{H}: 8.79 (bs, 1H, COOH), 4.58 (d, 1H, J=16.9 Hz, CHₐH_{b}COOH), 4.45-4.32 (m, 1H, CH₂CHO), 4.45 (d, 1H, J=16.9 Hz, CHaH,COOH), 2.50-2.06 (m, 4H, CH₂(CH₂)₂(CH₂)₂), 2.05-1.73 (m, 3H, CH₂CHₐH_{b}CH₂CO), 1.68-1.58 (m, 2H, CH₂CH₂CC), 1.49 (m, 1H, CH₂CHₐH_{b}CH₂CO). ¹³C NMR (100 MHz, CDCl₃, 25° C.) δ_{C}: 173.9 (COOH), 102.2 (CH₂CCCHO), 90.8 (CH₂CCCHO), 73.1 (CCHO), 65.6 (OCH₂COOH), 42.1 (CH₂CHO), 34.2 (CH₂CH₂CC), 29.5 (CH₂CH₂CH₂CC), 26.1 (CH₂CH₂CH₂CHO), 20.6 (CH₂CH₂CH₂CC). MS (ES⁻, DCM) m/z: 181.2 [M-H]⁻. HRMS calcd. for C₁₀H₁₄O₃ [M-H]⁻ 181.2170, found 181.2174.

### CMC-Cyclooctyne

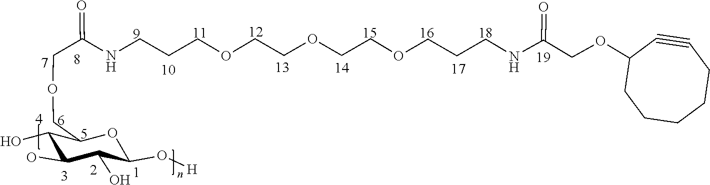

CMC-PEG₍ₙ₌₃)-NH₂ (DoS: 0.35-0.58) (33 mg, 0.09 mmol) was suspended in anhydrous dimethylformamide (1 mL) under a nitrogen atmosphere. A solution of (rac)-2-(cyclooct-2-yn-1-yloxy) acetic acid (20 mg, 0.11 mmol), O-(benzotriazol-1-yl)-N,N,N′,N′-tetramethyluronium hexafluorophosphate (HBTU, 42 mg, 0.11 mmol) and N,N-diisopropylethylamine (19 μL, 0.11 mmol) in anhydrous dimethylformamide (1.5 mL) was added to the suspension. The reaction mixture was stirred at room temperature overnight (16 h), before the product was filtered and washed with ethyl acetate (3×10 mL), acetone (3×10 mL) and water (10 mL) to afford the titled compound as a pale yellow compressible solid (38 mg, 70% mass recovery). Solid State ¹³C NMR (10,000 MHz CP MAS) δ_{C}: 176.21 (C-8 of Na-CMC), 171.37 (C-8, C-19), 130.52, 102.97 (C-1), 97.03, 81.25 (C-4), 74.14 (C-2, C-3, C-5), 70.39 (C-7), 61.39 (C-6), 36.53-28.19 (C-9, C-10, C-11, C-12, C-13, C-14, C-15, C-16, C-17, C-18. FTIR (vₘₐₓ/cm⁻¹) 3292 (O—H), 2871 (N—H, C—H), 1650 (CONH, amide of coupled product), 1589 (HNCOO, CMC), 1388 (COO), 1315 (ar. C═C bending), 1023 (CN, primary amine). Raman (vₘₐₓ/cm⁻¹) 2324 (CC, alkyne), 1780-1650 (C═O, CONH of amide coupled product and HNCOO of CMC), 1640-1540 (C—C, of CMC backbone).

### Example 5—Synthesis of CMC-Staudinger

### CMC-Staudinger (Route 1)

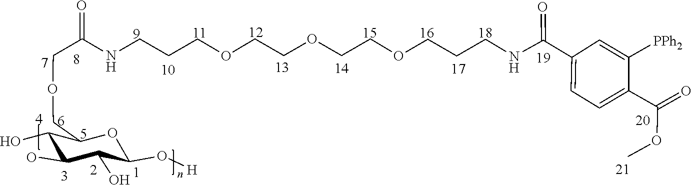

CMC-PEG₍ₙ₌₃₎-NH₂ (DoS: 0.35-0.58) (220 mg, 0.63 mmol) was suspended in dry dimethylformamide (3 mL) under a nitrogen atmosphere. A solution of 3-(diphenylphosphaneyl)-4-(methoxycarbonyl)benzoic acid (266 mg, 0.73 mmol), HBTU (277 mg, 0.73 mmol) and N,N-diisopropylethylamine (127 μL, 0.73 mmol) in dry dimethylformamide (3 mL) was added to the suspension. The reaction mixture was stirred at room temperature overnight (16 h), before the product was filtered and washed with ethanol (3×10 mL), acetone (3×10 mL) and water (10 mL). The product was concentrated in-vacuo to afford the titled compound as a pale red compressible solid (229 mg, 46% mass recovery). Solid State ¹³C NMR (10,000 MHz CP MAS) δ_{C}: 176.94 (C-8 of Na⁺-CMC, C-20), 171.81 (C-8, C-19), 131.54 (C_{Ar}), 103.10 (C-1), 96.79, 81.69 (C-4), 74.08 (C-2, C-3, C-5), 70.21 (C-7), 61.46 (C-6), 52.26 (C-21), 36.29-28.17 (C-9, C-10, C-11, C-12, C-13, C-14, C-15, C-16, C17, C-18). Solid State ³¹P NMR (11,000 MHz, CP MAS) δ_{P}: −4.39 (PPh₂). FTIR (vₘₐₓ/cm⁻¹): 3300 (O—H), 2871 (N—H, C—H), 1649 (CONH, amide of coupled product), 1591 (HNCOO, CMC). EA calculated C:59.54%, H:6.53%, N:3.56%, P:3.94% found C: 45.55%, H: 6.70%, N: 3.51%, P: 0.31%.

DoS by elemental analysis: 0.31% P wt/wt, corresponding to 0.0031 g/g. MW P=30.97 g/mol, so 0.0001 mol/g. or 0.1 mmol/g, DoC=0.1/1.3=0.077, so therefore DoS=(0.1/3.21)×0.7=0.022.

### CMC-Staudinger (Route 2 via Sulfo-NHS Activation)

N-Hydroxysulfosuccinimide (510 mg, 2.35 mmol), 3-(diphenylphosphaneyl)-4-(methoxycarbonyl) benzoic acid (430 mg, 1.18 mmol) and 1-(3-dimethylaminopropyl)-3-ethylcarbodiimide hydrochloride (452 mg, 2.36 mmol) were dissolved in anhydrous dimethylformamide (8 mL). The resulting solution was stirred at room temperature for 2 hours. On completion, the reaction mixture was concentrated under reduced pressure to afford a bright red oil which was immediately added to a 1% w/v suspension of CMC-PEG-NH₂ (DoS: 0.35-0.58) (520 mg, 1.18 mmol) in 520 mL of PBS buffer (pH 7.4). The resulting suspension was stirred at room temperature overnight (16 h) before the reaction mixture was lyophilized over several days to afford a dark red solid. The resulting solid was washed with dimethylformamide (50 mL), dichloromethane (100 mL) and acetone (100 mL) to afford the titled compound as a pale orange compressible solid (560 mg, 64%). Solid State ¹³C NMR (10,000 MHz CP MAS) δ_{C}: 176.94 (C-8 of Na-CMC, C-20), 171.81 (C-8, C-19), 131.54 (C_{Ar}), 103.10 (C-1), 96.79, 81.69 (C-4), 74.08 (C-2, C-3, C-5), 70.21 (C-7), 61.46 (C-6), 52.26 (C-21), 36.29-28.17 (C-9, C-10, C-11, C-12, C-13, C-14, C-15, C-16, C17, C-18). Solid State ³¹P NMR (11,000 MHz, CP MAS) δ_{P}: −4.39 (PPh₂). FTIR (vₘₐₓ/cm⁻¹): 3300 (O—H), 2871 (N—H, C—H), 1649 (CONH, amide of coupled product), 1591 (HNCOO, CMC). EA calculated C: 59.54%, H: 6.53%, N: 3.56%, P: 3.94% found C: 45.55%, H: 6.70%, N: 3.51%, P: 0.31%. DoS by elemental analysis: 1.1% P wt/wt, corresponding to 0.011 g/g. MW P=30.97 g/mol, so 0.00355 mol/g. or 0.355 mmol/g, DoC=0.355/1.3=0.27, therefore DoS=(0.355/3.21)×0.7=0.077.

DoS by Kaiser test: This assay gave 0.34 mmol/g available amine groups, so the reduction in available amine is 0.84 mmol/g; assume the conversion to Staudinger is 0.84 mmol/g. The amount of free amine on the CMC-PEG(n=3)-NH₂ is 1.18 mmol/g, so the DoC of reactive primary amine (available to the Kaiser test) into cyclooctyne is 0.71. The average DoS per D-anhydroglucopyranose monomers of cellulose for this step is 0.18 according to this measure.

### Examples 6 to 18—Synthesis of CMC-Biomolecule Compounds

The third step is to couple the CMC-reactive member compound with a biomolecule with an azide functional group as the complementary reactive member to form CMC-biomolecule compounds.

The CMC-reactive member compounds have been coupled to simple azide saccharides (Scheme 6). The CMC-biomolecule compounds derived from a coupling reaction with simple azide saccharides are CMC-F-Cyclooctyne-GlcNAc (Example 6), and CMC-Staundinger-GlcNAc (Example 7). The saccharide moiety may influence cell behaviour and interact with glycoproteins in the extracellular matrix (ECM).

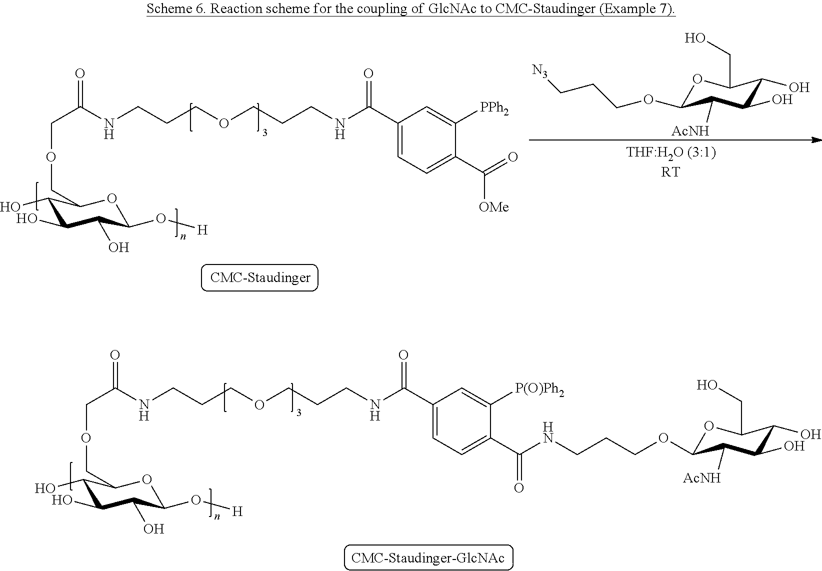

In addition, the CMC-reactive member compounds have been coupled to an azide derivative of the Zn(II) chelator TPA (3-azido-N,N-bis(pyridine-2-ylmethyl) propan-1-amine) (Scheme 7) (Nadler et al., 2009). This may provide a biomedical device that can regulate the levels of Zn(II) at the site of a wound, and in turn, modify the activity of MMPs in chronic wounds. The CMC-biomolecule compounds derived from a coupling reaction with an azide derivative of the Zn(II) chelator TPA include CMC-F-Cyclooctyne-Chelator (Example 8).

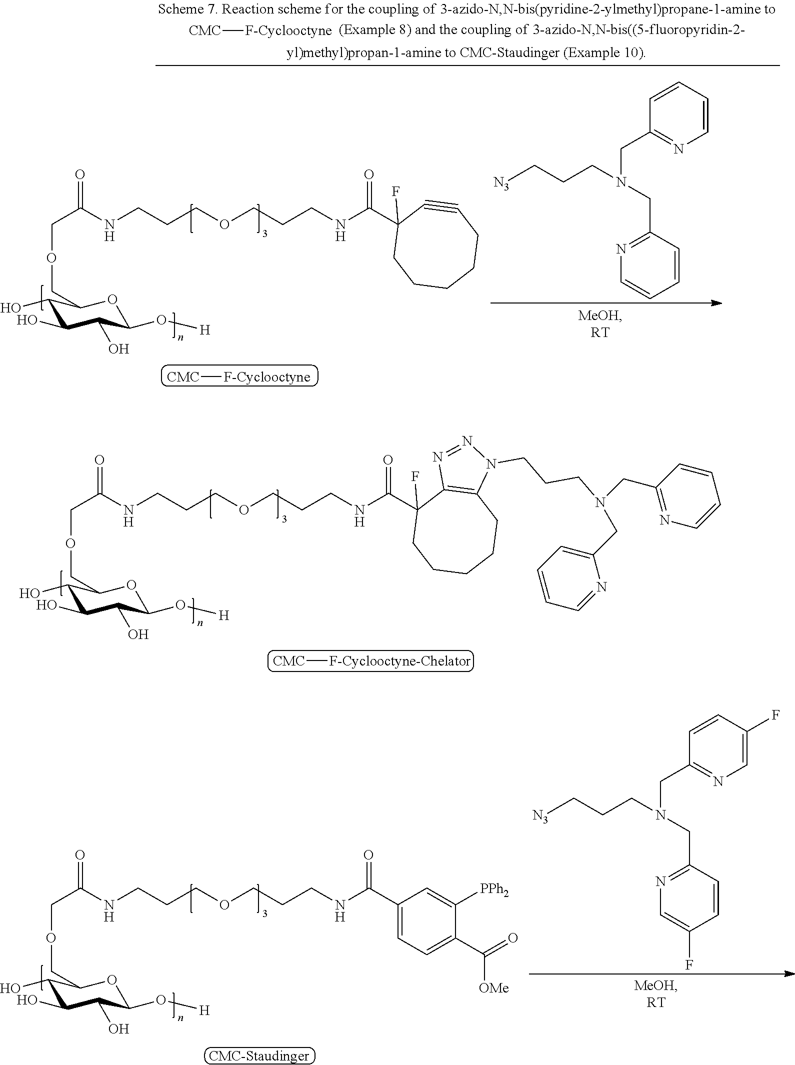

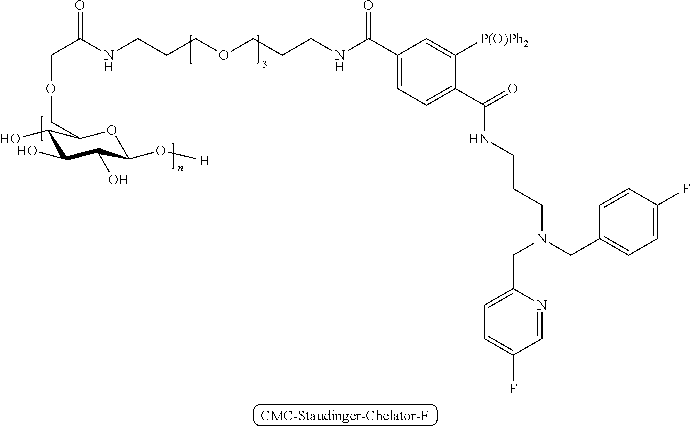

Furthermore, the CMC-reactive member compounds have been coupled to an azide derivative of various fluorescent probes or fluorophores (Scheme 8). The emission and absorbance of the fluorophores may be used to quantify the efficiency of the SPAAC and Staudinger ligations. Furthermore, the fluorophores may be used as fluorescent probes at the wound site. For example, fluorescein is a pH probe which when attached to the CMC surface may be able to report on wound pH (Le Guer et al., 2020; Bidmanova et al., 2012).

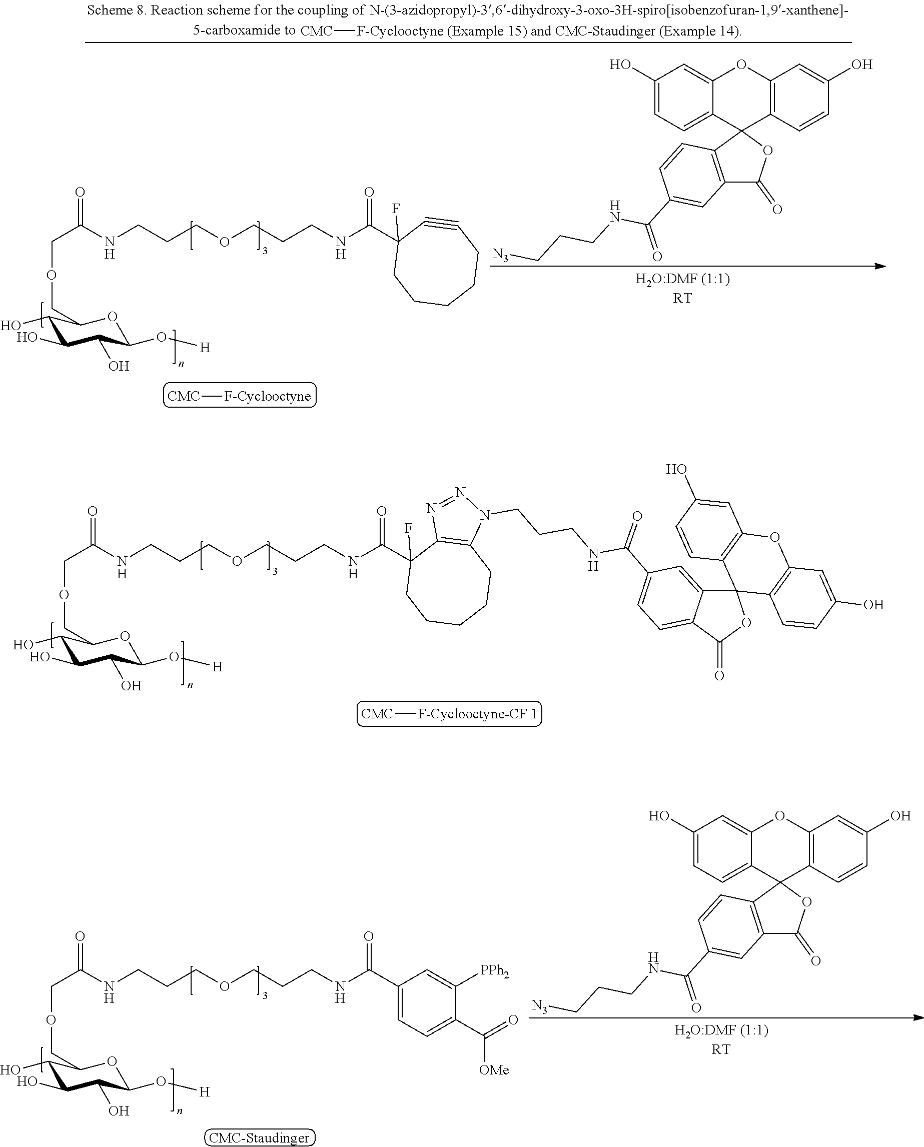

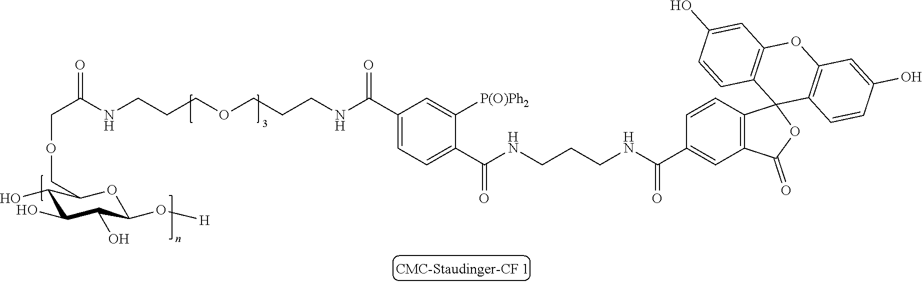

Additionally, the CMC-reactive member compounds have been coupled to an azide derivative of a peptide (Scheme 9). The peptide attached to the CMC derivative may be a signalling ligand that can alter cell behaviour, for example peptide-based analogues of native ECM components may promote cell adhesion and cell proliferation in or on these CMC hydrogels (Kharkar et al., 2013). Alternatively, the peptides may have antimicrobial activity that suppresses wound infection, for example the peptaibols are a class of membrane active antimicrobial peptide that contains high proportions of Aib (Daniel et al., 2007), as in Example 18.

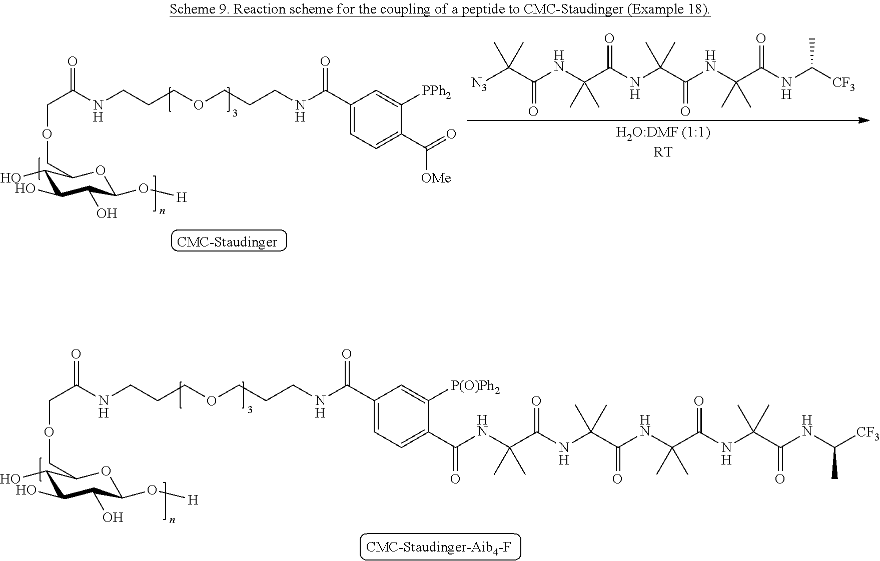

### Example 6—Synthesis of CMC-F-Cyclooctyne-GlcNAc

### CMC-F-Cyclooctyne-GlcNAc

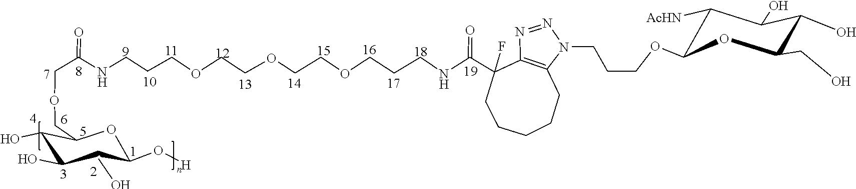

CMC-F-Cyclooctyne (100 mg, 0.17 mmol) was suspended in a solution of azidopropyl-β-D-GlcNAc (46 mg, 0.16 mmol) in methanol (2 mL). The resulting suspension was stirred at room temperature for 2 hours, before being filtered. The filtered solid was washed with methanol (2×10 mL), dichloromethane (2×10 mL) and acetone (2×10 mL) to afford the titled compound as a pale-yellow compressible solid (96 mg, 66% mass recovery). FTIR (vₘₐₓ/cm⁻¹) 3292 (O—H), 2871 (N—H, C—H), 1650 (CONH, amide of coupled product), 1589 (HNCOO, CMC), 1388 (COO), 1315 (aromatic C═C bending), 1023 (CN, primary amine). EA calculated C:50.33%, H:7.19%, N:9.52%, F:2.15% found C:42.94%, H:6.43%, N: 3.13%, F: N/A. Raman (vₘₐₓ/cm⁻¹) 1780-1650 (C═O, CONH of amide coupled product and HNCOO of CMC), 1640-1540 (C—C, of CMC backbone). No alkyne stretch.

### Example 7—Synthesis of CMC-Staudinger-GlcNAc

### CMC-Staudinger-GlcNAc

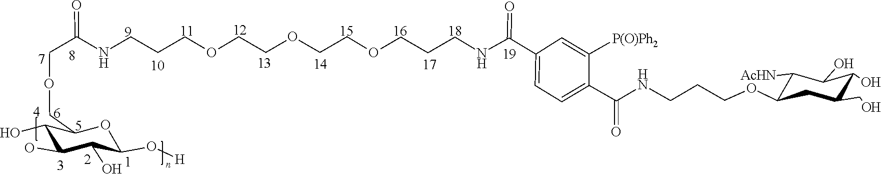

CMC-Staudinger (100 mg, 0.13 mmol) was suspended in a solution of azidopropyl-β-D-GlcNHAc (46 mg, 0.16 mmol) in a tetrahydrofuran: water (3:1) (3 mL) mixture. The resulting suspension was stirred at room temperature for 2 hours, before being filtered. The filtered solid was washed with methanol (2×10 mL), acetone (2×10 mL) and water (2×10 mL) to afford the titled compound as a pale-yellow compressible solid (102 mg, 78% mass recovery). Solid State ¹³C NMR (10,000 MHz CP MAS) δ_{C}: 176.21 (C-8 of Na-CMC), 171.37 (C-8, C-19), 130.52, 102.97 (C-1), 97.03, 81.25 (C-4), 74.14 (C-2, C-3, C-5), 70.39 (C-7), 61.39 (C-6), 36.53-28.19 (C-9, C-10, C-11, C-12, C-13, C-14, C-15, C-16, C-17, C-18. Solid State ³¹P NMR (11,000 MHz, CP MAS) δ_{P}: −41.08 (P(O)Ph₂). FTIR (vₘₐₓ/cm⁻¹) 3292 (O—H), 2871 (N—H, C—H), 1650 (CONH, amide of coupled product), 1589 (HNCOO, CMC), 1388 (COO), 1315 (ar. C═C), 1023 (CN, primary amine).

### Example 8—Synthesis of CMC-F-Cyclooctyne-Chelator

### 3-Azido-N,N-bis (pyridine-2-ylmethyl) propan-1-amine (Az-BPA)

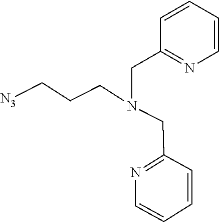 [SMILES: [N-]=[N+]=NCCCN(Cc1ccccn1)Cc1ccccn1]

To a solution of 2-azidopropylamine (213 mg, 2.48 mmol) in dichloromethane (5 mL) was added pyridinecarboxaldehyde (267 mg, 2.50 mmol). The reaction was stirred at room temperature for 30 mins before sodium triacetoxyborohydride (636 mg, 3.00 mmol) was slowly added. On completion, the reaction was stirred for an additional 3 h at room temperature. The reaction was then diluted with a mixture of dichloromethane: water (1:1) (20 mL), and the organic layer extracted, dried (MgSO₄) and concentrated under reduced pressure to afford the titled compound as a colourless oil (380 mg, 54%). ¹H NMR (400 MHz, CDCl₃, 25° C.) δ_{H}: 8.47 (d, 2H, J=4.4 Hz, (CₐᵣNCₐᵣH)₂), 7.60 (m, 2H, NCₐᵣHCₐᵣH), 7.42 (d, 2H, J=7.8 Hz, (CₐᵣCₐᵣH)₂), 7.09 (m, 2H, CₐᵣCₐᵣHCₐᵣH), 3.75 (s, 4H, N (CH₂)₂), 3.24 (t, 2H, J=6.8 Hz, CH₂N), 2.57 (t, 2H, J=6.8 Hz, N₃CH₂), 1.72 (p, 2H, J=6.8 Hz, CH₂CH₂CH₂). ¹³C NMR (100 MHz, CDCl₃, 25° C.) δ_{C}: 159.5 (2×NCₐᵣCₐᵣ), 149.1 (2×CₐᵣCH₂), 136.5 (2×NCₐᵣCₐᵣ), 122.9 (2×CₐᵣCₐᵣCH₂), 122.1 (2×CₐᵣCₐᵣCₐᵣCH₂), 60.5 (2×NCH₂Cₐᵣ), 51.2 (NCH₂CH₂), 49.4 (CH₂CH₂CH₂), 26.6 (CH₂N₃). MS (ES⁺, DCM) m/z: 293.4 [M-Na]⁺. HRMS calcd. for C₁₅H₁₈N₆ [M+Na]⁺ 293.4201, found 293.4171.

### CMC-F-Cyclooctyne-Chelator

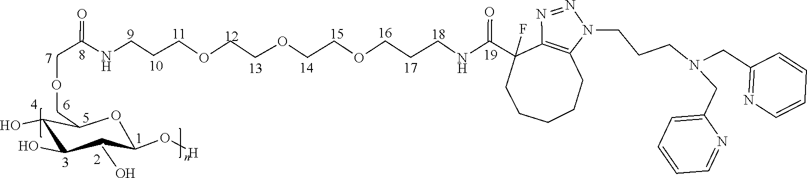

CMC-F-Cyclooctyne (100 mg, 0.17 mmol) was suspended in a solution of 3-azido-N,N-bis (pyridine-2-ylmethyl) propan-1-amine (46 mg, 0.16 mmol) in methanol (2 mL). The resulting suspension was stirred at room temperature for 2 h, before being filtered. The filtered solid was washed with methanol (2×10 mL), dichloromethane (2×10 mL) and acetone (2×10 mL) to afford the titled compound as a pale-yellow compressible solid (112 mg, 68% mass recovery). FTIR (vₘₐₓ/cm⁻¹) 3292 (O—H), 2871 (N—H, C—H), 1650 (CONH, amide of coupled product), 1589 (HNCOO, CMC), 1388 (COO), 1315 (aromatic C═C bending), 1023 (CN, primary amine). EA calculated C:57.65%, H:7.26%, N:12.81%, F:2.17% found C:43.81%, H:6.83%, N:3.48%, F: N/A. Raman (vₘₐₓ/cm⁻¹) 1780-1650 (C═O, CONH of amide coupled product and HNCOO of CMC), 1640-1540 (C—C, of CMC backbone). No alkyne stretch.

### Example 9—Synthesis of CMC-F-Cyclooctyne-Chelator-F

### 3-Azido-N,N-bis((5-fluoropyridin-2-yl) methyl)propan-1-amine

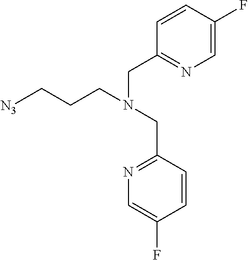 [SMILES: [N-]=[N+]=NCCCN(Cc1ccc(F)cn1)Cc1ccc(F)cn1]

To a solution of 3-azidopropylamine (71 mg, 0.71 mmol) in dichloromethane (5 mL) was added 5-fluoro-2-formylpyridine (104 mg, 0.83 mmol). The reaction was stirred at room temperature for 30 mins before sodium triacetoxyborohydride (212 mg, 1.00 mmol) was slowly added. On completion, the reaction was stirred for an additional 3 h at room temperature. The reaction was then diluted with a mixture of dichloromethane:water (1:1) (20 mL), and the organic layer extracted, dried (MgSO₄) and concentrated under reduced pressure to afford the titled compound as a colourless oil (91 mg, 40%). ¹H NMR (400 MHz, CDCl₃, 25° C.) δ_{H}: 8.41 (d, 2H, J=2.8 Hz, 2×NCₐᵣHCₐᵣF), 7.47 (dd, 2H, J=8.6 Hz, 2×CₐᵣFCₐᵣHCₐᵣH), 7.40 (td, 2H, J=2.9 Hz, 2×CₐᵣFCₐᵣHCₐᵣH), 3.80 (s, 4H, N(CH₂)₂), 3.33 (t, 2H, J=6.7 Hz, N₃CH₂), 2.64 (t, 2H, J=6.9 Hz, N(CH₂)₂CH₂), 1.80 (p, 2H, J=13.7 Hz, N₃CH₂CH₂). ¹⁹F NMR (376 MHz, CDCl₃, 25° C.) δ_{F}: −129.5 (s). MS (ES⁺, DCM) m/z: 341.3 [M-Na]⁺. HRMS calcd. for C₁₅H₁₆N₆F₂ [M+Na]⁺ 341.1297, found 341.1285.

### CMC-F-Cyclooctyne-Chelator-F

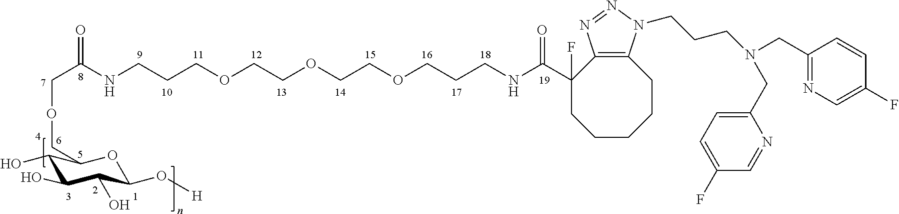

CMC-F-Cyclooctyne (100 mg, 0.17 mmol) was suspended in a solution of 3-azido-N,N-bis((5-fluoropyridin-2-yl)methyl)propan-1-amine (45 mg, 0.16 mmol) in methanol (2 mL). The resulting suspension was stirred at room temperature for 2 h, before being filtered. The filtered solid was washed with methanol (2×10 mL), dichloromethane (2×10 mL) and acetone (2×10 mL) to afford the titled compound as a pale-yellow compressible solid (101 mg, 65% mass recovery). Solid State ¹³C NMR: (6,000 MHz CP MAS, adamantane standard) δ_{C}: 174.21 (C8 of unreacted CH₂CO₂Na), 169.37 (C8, C19), 128.47, 100.47 (C1), 95.98, 79.44 (C4), 71.14 (C2, C3, C5), 68.96 (C7), 58.97 (C6), 36.53-28.19 (C9, C10, C11, C12, C13, C14, C15, C16, C17, C18). FTIR (vₘₐₓ/cm⁻¹) 3292 (O—H), 2871 (N—H, C—H), 1650 (CONH, amide of coupled product), 1589(HNCOO, CMC), 1388 (COO), 1315 (aromatic C═C bending), 1023 (CN, primary amine). EA calculated C:57.65%, H:7.26%, N:12.81%, F:2.17% found C:43.81%, H:6.83%, N:3.48%, F: N/A. Raman (vₘₐₓ/cm⁻¹) 1780-1650 (C═O, CONH of amide coupled product and HNCOO of CMC), 1640-1540 (C—C, of CMC backbone). No alkyne stretch.

DoS by elemental analysis: <0.50% F wt/wt, corresponding to <0.005 g/g. MW F=19 g/mol, so <0.0002 mol/g. Therefore <0.2 mmol/g. DoC can't be calculated. DoS=(<0.2/3.21)×0.7=<0.044.

### Example 10—Synthesis of CMC-Staudinger-Chelator-F

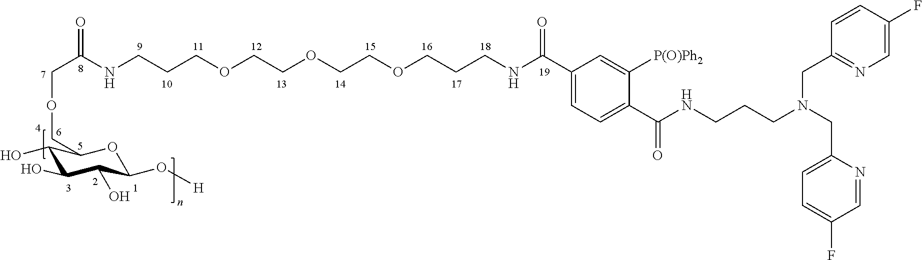

CMC-Staudinger (100 mg, 0.13 mmol) was suspended in a solution of 3-azido-N,N-bis ((5-fluoropyridin-2-yl) methyl) propan-1-amine (45 mg, 0.16 mmol) in methanol (2 mL). The resulting suspension was stirred at room temperature for 2 h, before being filtered. The filtered solid was washed with methanol (2×10 mL), dichloromethane (2×10 mL) and acetone (2×10 mL) to afford the titled compound as a pale-yellow compressible solid (96 mg, 70% mass recovery). Solid State ¹⁹F NMR (14,000 MHz, CP MAS, C₆F₆ standard) δ_{F}: −128.9 (2×Cₐᵣ—F). FTIR (vₘₐₓ/cm⁻¹): 3431 (O—H), 2936 (N—H, C—H), 1708 (CONH, amide of coupled product), 1625 (CO of unreacted CH₂CO₂Na), 1438 (COO), 1046 (CN, primary amine). Elemental analysis: calculated C: 59.88%, H: 6.16%, N: 7.91%, P: 2.91%; found C: 26.37%, H: 3.96%, N: 5.23%, P: 0.93%. Raman (vₘₐₓ/cm⁻¹) 1780-1650 (C═O, CONH of amide coupled product and HNCOO of CMC), 1640-1540 (C—C, of CMC backbone).

### Example 11—Synthesis of CMC-Staudinger-Chelator-Quinoline

### 3-Azido-N-(isoquinolin-3-ylmethyl)-N-(quinoline-2-ylmethyl)propan-1-amine

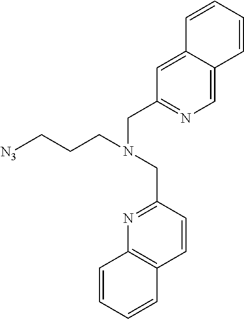 [SMILES: [N-]=[N+]=NCCCN(Cc1cc2ccccc2cn1)Cc1ccc2ccccc2n1]

To a flame-dried round bottom flask was added 3-azidopropylamine (100 mg, 1.00 mmol) and 2-quinolinecarboxaldehyde (629 mg, 4.00 mmol) to anhydrous-dichloromethane (12 mL) with activated molecular sieves (3 μm, 20% w/v) under an argon atmosphere. The resulting solution was stirred at room temperature for 30 mins before sodium triacetoxyborohydride (636 mg, 3 mmol) was slowly added. On completion, the resulting reaction mixture was stirred at room temperature for 16 h before being diluted with dichloromethane (12 mL). The resulting organic layer was washed with saturated sodium hydrogen carbonate (2×12 mL), extracted, dried (Na₂SO₄) and concentrated under reduced pressure to afford a brown oil. The titled compound was afforded as an orange oil (188 mg, 47%) by column chromatography (silica gel, 0-5% methanol/chloroform). ¹H NMR (400 MHz, CDCl₃, 25° C.) δ_{H}: 8.07 (d, 1H, J=8.6 Hz, 2×CH₂CₐᵣNCₐᵣCₐᵣH), 7.98 (d, 1H, J=7.9 Hz, 2×CH₂CₐᵣCₐᵣHCₐᵣH), 7.72 (d, 1H, J=9.9 Hz, 2×CₐᵣHCₐᵣHCₐᵣCₐᵣH), 7.62 (m, 2H, 2×CₐᵣCₐᵣHCₐᵣHCₐᵣH), 7.45 (m, 1H, 2×CH₂CₐᵣCₐᵣH), 3.96 (s, 4H, 2×NCH₂Cₐᵣ) 3.23 (t, 2H, J=6.8 Hz, CH₂CH₂N), 2.66 (t, 2H, J=6.8 Hz, N₃CH₂), 1.76 (p, 2H, J=13.7 Hz, CH₂CH₂CH₂). ¹³C NMR (100 MHz, CDCl₃, 25° C.) δ_{C}: ¹³C NMR (100 MHz, CDCl₃, 25° C.) δ_{C}: 160.2 (2×CH₂CₐᵣN), 147.6 (2×CH₂CₐᵣNCₐᵣ), 136.4 (2×CH₂CₐᵣCₐᵣHCₐᵣH), 129.5, 129.1, 127.6, 126.2, 121.0, 61.5 (N(CH₂)₂), 51.6 (N₃CH₂CH₂CH₂), 49.5 (N₃CH₂CH₂CH₂), 25.7 (N₃CH₂CH₂CH₂).

### CMC-Staudinger-Chelator-Quinoline

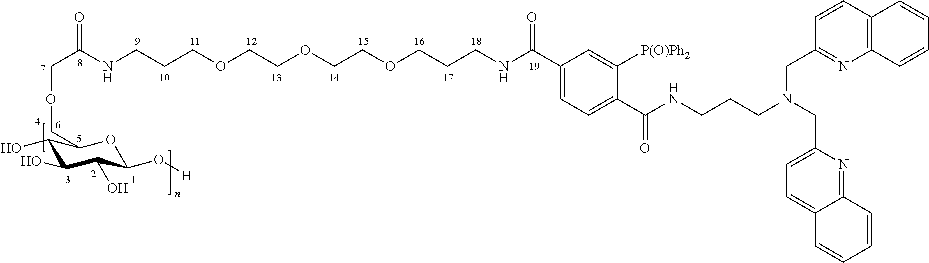

CMC-Staudinger (100 mg, 0.13 mmol) was suspended in a solution of 3-azido-N-(isoquinolin-3-ylmethyl)-N-(quinoline-2-ylmethyl)propan-1-amine (61 mg, 0.16 mmol) in methanol (2 mL). The resulting suspension was stirred at room temperature for 2 h, before being filtered. The filtered solid was washed with methanol (2×10 mL), dichloromethane (2×10 mL) and acetone (2×10 mL) to afford the titled compound as a pale-yellow compressible solid (109 mg, 75% mass recovery). FTIR (vₘₐₓ/cm⁻¹): 3290 (O—H), 2873 (N—H, C—H), 1644 (CONH, amide of coupled product), 1593 (CO of unreacted CH₂CO₂Na), 1054 (CN, primary amine). Elemental analysis calculated C: 6.00%, H: 6.35%, N: 7.46%, P: 3.67% found C: 35.43%, H: 5.91%, N: 2.60%, P: 2.36%. Fluorescence: λₑₓ=310±23 nm gives λₑₘ=458 nm. Raman (vₘₐₓ/cm⁻¹) 1780-1650 (C═O, CONH of amide coupled product and HNCOO of CMC), 1640-1540 (C—C, of CMC backbone).

### Example 12—Synthesis of CMC-Staudinger-CF₂

### 5(4)-((3-azidopropyl)carbamoyl)-2-(6-hydroxy-3-oxo-3H-xanthen-9-yl)benzoic acid (1:1)

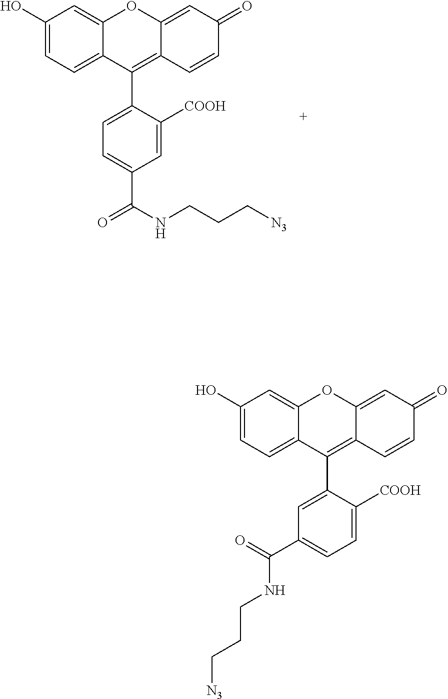 [SMILES: [N-]=[N+]=NCCCNC(=O)c1ccc(-c2c3ccc(=O)cc-3oc3cc(O)ccc23)c(C(=O)O)c1 ; [N-]=[N+]=NCCCNC(=O)c1ccc(C(=O)O)c(-c2c3ccc(=O)cc-3oc3cc(O)ccc23)c1]

To a solution of (5)6-carboxyfluorescein N-hydroxysuccinimide ester (100 mg, 0.21 mmol) in DMF (2 mL) was added 3-azido-1-propanamine (25 mg, 0.25 mmol) and DIPEA (98 μL, 0.63 mmol). The resulting solution was stirred at room temperature overnight (16 h) and covered in foil to protect from light. On completion, the reaction mixture was concentrated under reduced pressure to afford a bright yellow solid. The titled compounds were afforded as a mixture by column chromatography (silica gel, DCM: MeOH: AcOH (95:5:0.5)) (34 mg, 35%). ¹H NMR (400 MHz, CDCl₃, 25° C.) δ_{H}: 8.44 (s, 1H), 8.21 (d, 1H, J=8.0 Hz), 8.13 (d, 1H, J=8.0 Hz), 8.08 (d, 1H, J=8.0 Hz), 7.63 (s, 1H), 7.31 (d, 1H, 8.0 Hz), 6.71 (m, 4H), 6.61 (m, 4H), 6.56 (m, 4H), 3.53 (t, 2H, J=6.8 Hz), 3.46 (t, 2H, J=6.7 Hz), 3.40 (t, 2H, J=6.8 Hz), 3.34 (t, 2H, J=6.6 Hz), 1.93 (p, 2H, J=13.6, 6.7 Hz), 1.81 (p, 2H, J=13.4, 6.7 Hz).

### CMC-Staudinger-CF₂

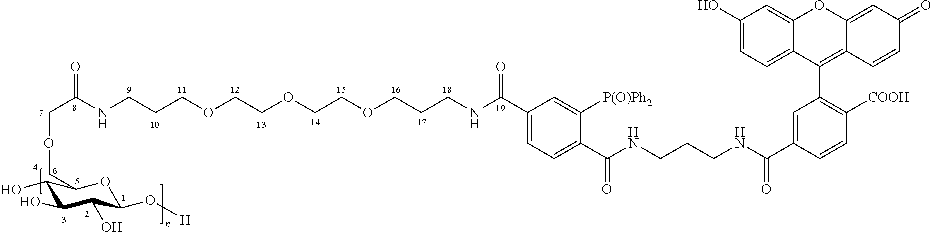

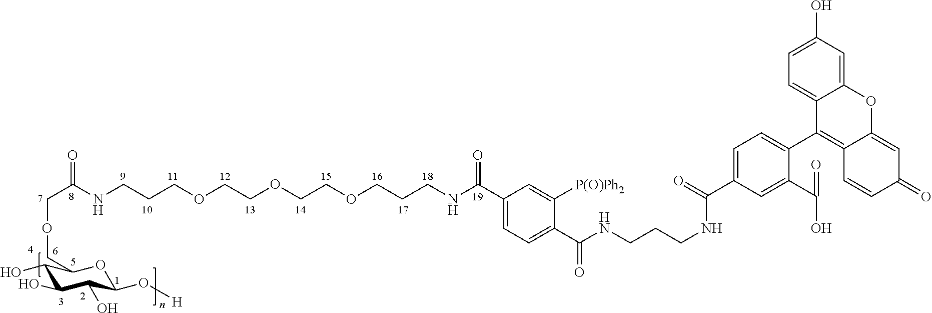

CMC-Staudinger (17 mg, 0.22×10⁻¹ mmol) was suspended in 1.7 mL of PBS buffer (pH 7.4) to make a 1% w/v solution. The resulting suspension was stirred at room temperature for 3 hours. On completion, 5(4)-((3-azidopropyl)carbamoyl)-2-(6-hydroxy-3-oxo-3H-xanthen-9-yl)benzoic acid (10 mg, 0.2×10⁻¹ mmol) in 1.7 mL of DMSO was added. The resulting suspension was covered in tin foil to protect from light and stirred at room temperature overnight (16 h). On completion, the water was removed under reduced pressure and the resulting solid was collected by filtration and washed with DMSO (2×20 mL), acetone (2×20 mL) and DCM (2×20 mL) to afford a bright yellow compressible solid (14 mg, 53% mass recovery, 1:1 ratio). Fluorescence: λₑₓ of 456±23 nm gives λₑₘ=517 nm.

### Example 13—Synthesis of CMC-F-Cyclooctyne-CF₂

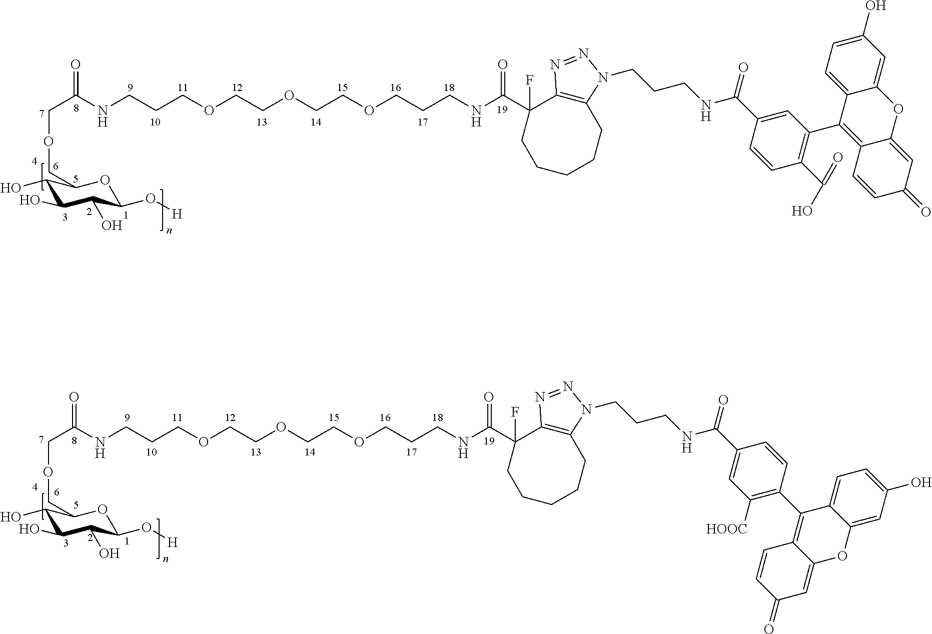

CMC-F-Cyclooctyne (13 mg, 0.22×10-1 mmol) was suspended in 1.3 mL of PBS buffer (pH 7.4) to make a 1% w/v solution. The resulting suspension was stirred at room temperature for 3 hours. On completion, 5(4)-((3-azidopropyl)carbamoyl)-2-(6-hydroxy-3-oxo-3H-xanthen-9-yl)benzoic acid (10 mg, 0.2×10⁻¹ mmol) in 1.3 mL of DMSO was added. The resulting suspension was covered in tin foil to protect from light and stirred at room temperature overnight (16 h). On completion, the water was removed under reduced pressure and the resulting solid was collected by filtration and washed with DMSO (2×20 mL), acetone (2×20 mL) and DCM (2×20 mL) to afford a bright yellow compressible solid (9 mg, 41% mass recovery, 1:1 ratio). Fluorescence: λₑₓ=456±23 nm gives λₑₘ=517 nm.

### Example 14—Synthesis of CMC-Staudinger-CF 1

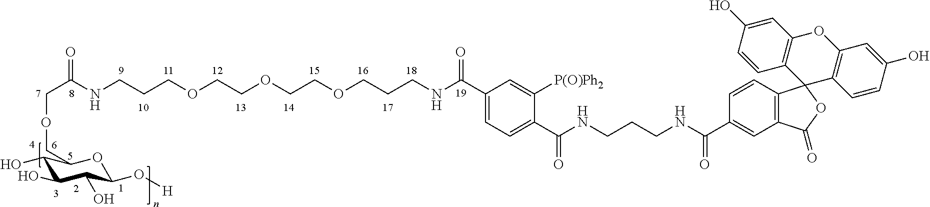

CMC-Staudinger (17 mg, 0.22×10⁻¹ mmol) was suspended in 1.7 mL of PBS buffer (pH 7.4) to make a 1% w/v solution. The resulting suspension was stirred at room temperature for 3 hours. On completion, N-(3-azidopropyl)-3′,6′-dihydroxy-3-oxo-3H-spiro[isobenzofuran-1,9′-xanthene]-5-carboxamide (10 mg, 0.22×10⁻¹ mmol) in 1.7 mL of DMSO was added. The resulting suspension was covered in tin foil to protect from light and stirred at room temperature overnight (16 h). On completion, the water was removed under reduced pressure and the resulting solid was collected by filtration and washed with DMSO (2×20 mL), acetone (2×20 mL) and DCM (2×20 mL) to afford a bright yellow compressible solid (14 mg, 54% mass recovery). Fluorescence: λₑₓ=494±4 nm gives λₑₘ=528 nm.

### Example 15—Synthesis of CMC-F-Cyclooctyne-CF 1

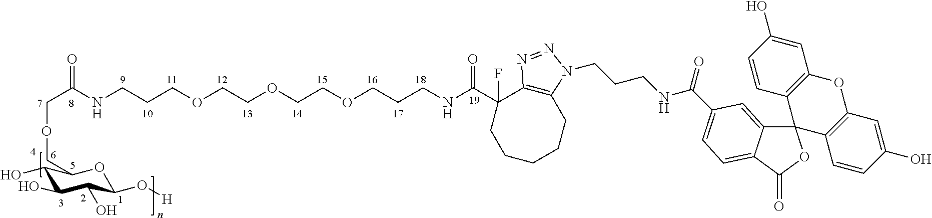

CMC-F-Cyclooctyne (13 mg, 0.22×10-1 mmol) was suspended in 1.3 mL of PBS buffer (pH 7.4) to make a 1% w/v solution. The resulting suspension was stirred at room temperature for 3 hours. On completion, N-(3-azidopropyl)-3′,6′-dihydroxy-3-oxo-3H-spiro [isobenzofuran-1,9′-xanthene]-5-carboxamide (10 mg, 0.22×10-1 mmol) in 1.3 mL of DMSO was added. The resulting suspension was covered in tin foil to protect from light and stirred at room temperature overnight (16 h). On completion, the water was removed under reduced pressure and the resulting solid was collected by filtration and washed with DMSO (2×20 mL), acetone (2×20 mL) and DCM (2×20 mL) to afford a bright yellow compressible solid (13 mg, 57% mass recovery). Fluorescence: λₑₓ=494±4 nm gives λₑₘ=528 nm.

### Example 16—Synthesis of CMC-F-Cyclooctyne-Bene

### 6-chloro-2-ethyl-1H-benzo[de]isoquinoline-1,3(2H)-dione

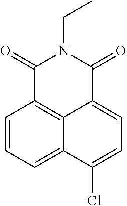 [SMILES: CCN1C(=O)c2cccc3c(Cl)ccc(c23)C1=O]

6-Chloro-1H,3H-benzo[de]isochromene-1,3-dione (200 mg, 0.86 mmol) and ethylamine (82 μL, 1.25 mmol) were suspended in 1,4-dioxane (100 mL). The resulting suspension was stirred and heated at reflux overnight (16 h). On completion, the suspension was cooled to room temperature before being poured onto ice water (300 mL) to afford a white precipitate. After the ice has melted, the precipitate was collected by filtration. The resulting solid was then washed from the filter paper into a separating funnel using DCM. The resulting solution then washed (brine), separated, dried (MgSO₄), filtered and then concentrated under reduced pressure to afford the titled compound as a white solid (129 mg, 58%). ¹H NMR (400 MHz, CDCl₃, 25° C.) δ_{H}: 8.59 (dd, 1H, J=7.3, 1.2 Hz, NCOCₐᵣCₐᵣHCₐᵣHCₐᵣH), 8.51 (dd, 1H, J=8.0 Hz, NCOCₐᵣCₐᵣHCₐᵣHCₐᵣCl), 8.43 (d, 1H, J=7.9 Hz, NCOCₐᵣCₐᵣHCₐᵣHCₐᵣH), 7.65 (dd, 1H, J=8.5, 7.3 Hz, NCOCₐᵣCₐᵣHCₐᵣHCₐᵣH), 7.37 (d, 1H, J=8.0 Hz, NCOCₐᵣCₐᵣHCₐᵣHCₐᵣCl), 4.16 (q, 2H, J=4.2 Hz, NCH₂CH₃), 1.26 (t, 3H, J=7.1 Hz, NCH₂CH₃).

### 6-Azido-2-ethyl-1H-benzo[de]isoquinoline-1,3(2H)-dione

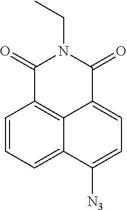 [SMILES: CCN1C(=O)c2cccc3c(N=[N+]=[N-])ccc(c23)C1=O]

To a round bottomed flask was added 6-chloro-2-ethyl-1H-benzo [de] isoquinoline-1,3 (2H)-dione (223 mg, 0.91 mmol) and sodium azide (293 mg, 4.51 mmol) and N-methylpyrrolidinone (7 mL). The resulting solution was then heated to 110° C. for 1.5 hr. On completion the solution was cooled to room temperature and then diluted with distilled water (25 mL) before being extracted with ethyl acetate (3×20 mL). The combined organic phases were then washed with brine, extracted, dried (MgSO₄), filtered and then concentrated under reduced pressure to afford a brown waxy solid. The titled compound was afforded as a yellow solid by column chromatography (silica gel, hexanes: ethyl acetate (4:1)) (136 mg, 56%). ¹H NMR (400 MHz, CDCl₃, 25° C.) δ_{H}: 8.55 (dd, 1H, J=7.3, 1.2 Hz, NCOCₐᵣCₐᵣHCₐᵣHCₐᵣH), 8.49 (d, 1H, J=8.0 Hz, NCOCₐᵣCₐᵣHCₐᵣHCₐᵣN₃), 8.34 (dd, 1H, J=8.4, 1.2 Hz, NCOCₐᵣCₐᵣHCₐᵣHCₐᵣH), 7.65 (dd, 1H, J=8.5, 7.3 Hz, NCOCₐᵣCₐᵣHCₐᵣHCₐᵣH), 7.37 (d, 1H, J=8.0 Hz, NCOCₐᵣCₐᵣHCₐᵣHCₐᵣN₃), 4.16 (q, 2H, J=4.2 Hz, NCH₂CH₃), 1.26 (t, 3H, J=7.1 Hz, NCH₂CH3).

### CMC-F-Cyclooctyne-Bene

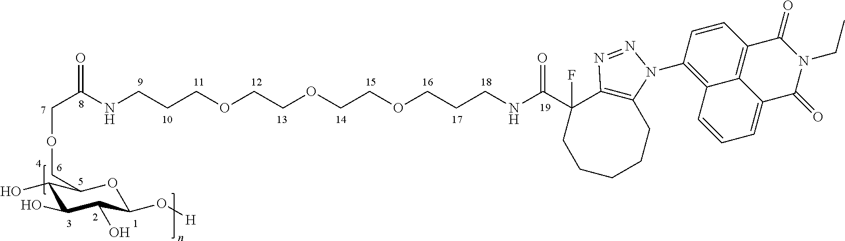

CMC-F-Cyclooctyne (46 mg, 0.78×10⁻¹ mmol) was suspended in 4.6 mL of PBS buffer (pH 7.4) to make a 1% w/v solution. The resulting suspension was stirred at room temperature for 3 hours. On completion, 6-azido-2-ethyl-1H-benzo[de]isoquinoline-1,3(2H)-dione (22 mg, 0.83×10⁻¹ mmol) in 4.6 mL of DMSO was added. The resulting suspension was stirred at room temperature overnight (16 h), before the water was removed under reduced pressure and the resulting solid was collected by filtration and washed with DMSO (2×20 mL), acetone (2×20 mL) and DCM (2×20 mL) to afford a bright yellow compressible solid (45 mg, 69% mass recovery). FTIR (vₘₐₓ/cm⁻¹): 3340 (O—H), 2922 (N—H, C—H), 1650 (CONH, amide of coupled product), 1590 (CO of unreacted CH₂CO₂Na), 1427 (COO), 1054 (CN, primary amine). Elemental analysis: calculated C: 57.33%, H: 6.45%, N: 9.78% found C: 11.21%, H: 2.19%, N: 1.25%. Fluorescence: λₑₓ=365±4 nm gives λₑₘ=533 nm.

### Example 17—Synthesis of CMC-Staudinger-Bene

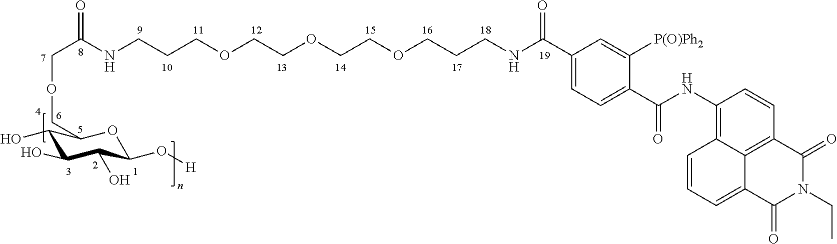

CMC-Staudinger (61 mg, 0.78×10-1 mmol) was suspended in 6.1 mL of PBS buffer (pH 7.4) to make a 1% w/v solution. The resulting suspension was stirred at room temperature for 3 hours. On completion, 6-azido-2-ethyl-1H-benzo[de]isoquinoline-1,3(2H)-dione (22 mg, 0.83×10⁻¹ mmol) in 6.1 mL of DMSO was added. The resulting suspension was stirred at room temperature overnight (16 h), before the water was removed under reduced pressure and the resulting solid was collected by filtration and washed with DMSO (2×20 mL), acetone (2×20 mL) and DCM (2×20 mL) to afford a bright yellow compressible solid (49 mg, 63% mass recovery). Fluorescence: λₑₓ=365±4 nm gives λₑₘ=537 nm.

### Example 18—CMC-Staudinger-Aib₄-F

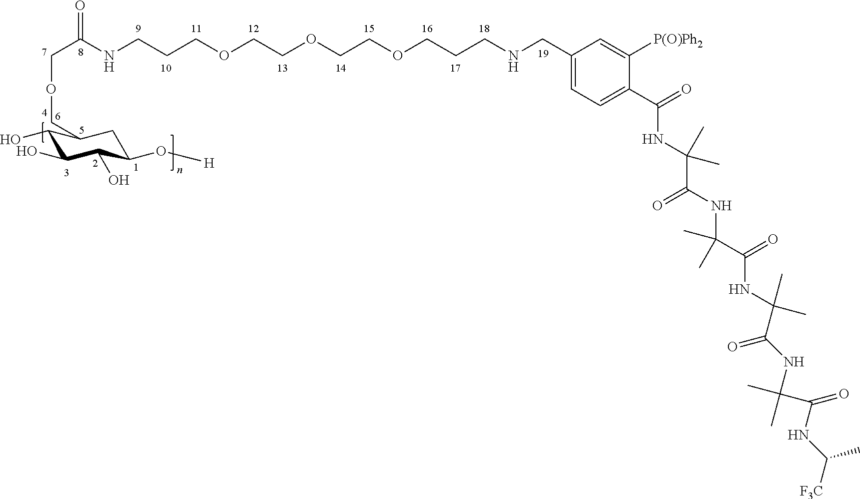

CMC-Staudinger (100 mg, 0.13 mmol) was suspended in distilled water (10 mL) to make a 1% w/v suspension and stirred at room temperature for 2 h. On completion, (R)-2-azido-2-methyl-N-(2-methyl-1-((2-methyl-1-((2-methyl-1-oxo-1-((1,1,1-trifluoropropan-2-yl)amino)propan-2-yl)amino)-1-oxopropan-2-yl) amino)-1-oxopropan-2-yl)propenamide (62 mg,0.13 mmol) in dimethylformamide (10 mL) was added to the suspension and the resulting mixture was stirred at room temperature for 16 h. On completion, the reaction mixture was concentrated under reduced pressure to afford a pale-yellow solid. The resulting pale-yellow solid was washed with dimethylformamide (2×20 mL), dichloromethane (2×20 mL), acetone (2×20 mL) and ethyl acetate (2×20 mL) to afford the titled compound as a pale-yellow compressible solid (106 mg, 66% mass recovery). Solid State ¹⁹F NMR (14,000 MHz, CP MAS, C₆F₆ reference) δ_{F}: −80.0 (CF3). FTIR (vₘₐₓ/cm⁻¹): 3290 (O—H), 2872 (N—H, C—H), 1650 (CONH, amide of coupled product), 1590 (CO of unreacted CH₂CO₂Na), 1388 (COO), 1350 (CF₃), 1023 (CN, primary amine). Elemental analysis: calculated C: 55.92%, H: 6.67%, N: 8.01%, P: 2.53%; found C: 12.63%, H: 2.19%, N: 1.42%, P: 1.42%.

### REFERENCES

A number of publications are cited above in order to more fully describe and disclose the invention and the state of the art to which the invention pertains. Full citations for these references are provided below. The entirety of each of these references is incorporated herein.
Abrigo et al., Macromolecular Bioscience, 2014, 14, 6, 772-792.
Agard et al., Journal of the American Chemical Society, 2004, 126, 15046-15047.
Barbucci et al., J. Mater. Chem. A, 2005, 15, 2234-2241.
Bidmanova et al., Sensors and Actuators B: Chemical, 2012, 161 (1), 93-99.
Codelli et al., Journal of the American Chemical Society, 2008, 130, 11486-11493.
Daniel et al., Nat. Prod. Rep., 2007, 24, 1128-1141.
Dunnill et al., International Wound Journal, 2017, 14, 89-96.
Ernsting et al., Bioconjugate Chemistry, 2011, 22, 2474-2486.
Falanga et al., Wound Repair and Regeneration, 1999, 7, 201-207.
Francesko et al., Biofunctionalization of Polymers and their Applications, eds. G. S. Nyanhongo, W.
Steiner and G. M. Gübitz, Springer, 2011, vol. 125, pp. 1-27.
Keppler et al., Glycobiology, 2001, 11, 11R-18R.
Kharkar et al., Chem. Soc. Rev., 2013, 42, 7335-7372.
Kolb et al., Angewandte Chemie International Edition, 2001, 40, 2004-2021.
Kolb et al., Drug Discovery Today, 2003, 8, 1128-1137.
Laughlin et al., Methods in Enzymology, 2006, 415, 230-250.
Le Guern et al., Int. J. Mol. Sci., 2020, 21 (23), 9217.
Liu et al., Diabetes Care, 2009, 32, 117-119.
Mehmood et al., Journal of Biomedical Materials Research Part B: Applied Biomaterials, 2014, 102, 4, 885-895.
Menke et al., Clinics in Dermatology, 2007, 25, 19-25.
Miller et al., Nature Chemical Biology, 2007, 3, 263-267.
Nadler et al., European Journal of Organic Chemistry, 2009, 4593-4599.
O'Sullivan et al., Cellulose, 1997, 4, 173-207.
Pettignano et al., Polymer Reviews, 2019, 59, 3, 510-560.
Puerta et al., Journal of the American Chemical Society, 2004, 126, 8388-8389.
Queen et al., Wounds International, 2011, 2, 1-6.
Saxon et al., Organic Letters, 2000b, 2, 2141-2143.
Saxon et al., Science, 2000a, 287, 2007-2010.
Shelke et al., Polym. Adv. Technol., 2014, 25, 448-460.
Sletton et al., Organic Letters, 2008, 10, 14, 3097-3099.
Soneja et al., Pharmacological Reports, 2005, 57, 108.
Soyez et al., Advanced Drug Delivery Reviews, 1996, 21, 81-106.
Stewart, Nature, 1981, 292, 17-21.
Srikun et al., Journal of the American Chemical Society, 2008, 130, 4596-4597.
Staudinger et al., Helvetica Chimica Acta, 1919, 2, 635-646.
U.S. Pat. No. 8,512,728 B2
U.S. Pat. No. 9,180,221 B2
U.S. Pat. No. 9,510,810 B2
Wang et al., Sensors and Actuators B: Chemical, 2016, 228, 174-179.
Ward et al., Frontiers in Bioengineering and Biotechnology, 2020, 8, 1039.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

1. A method of functionalising sodium carboxymethylcellulose (Na-CMC), which is a cellulose comprising at least one unit of Formula 0: 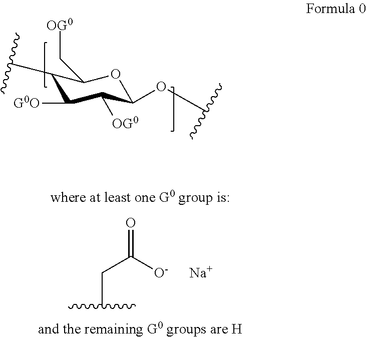
the method comprising the steps of:
i) coupling at least one said unit of Formula 0 with a spacer selected from: 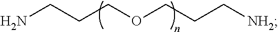
where n is an integer from 0 to 450; to form a CMC derivative comprising at least one unit of Formula 1: 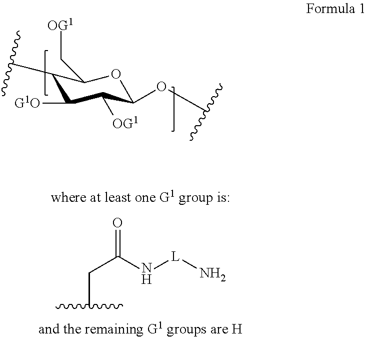
wherein L is: 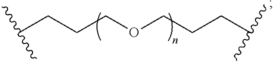
derived from the spacer;
ii) then coupling at least one unit of Formula 1 in said CMC derivative with a reactive member reagent selected from: 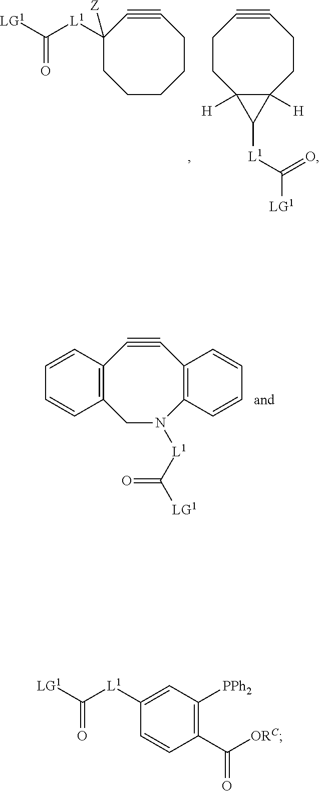
wherein:
LG¹ is a leaving group;
L¹ is a bond, alkylene, -(alkylene)O—*, —O(alkylene)-*, or -(alkylene)C(O)—*;
wherein the*is the attachment to the cyclooctyne or benzene ring;
Z is —F, —Cl, —Br, or —I; and
R^{C} is alkyl;
to form a CMC derivative comprising at least one unit of Formula 2: 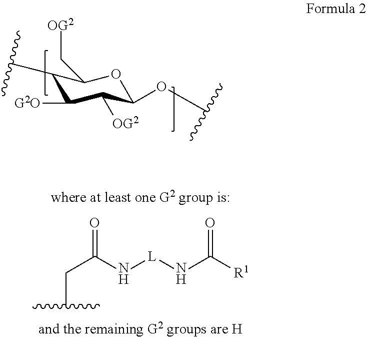
wherein:
R¹ is a reactive member selected from: 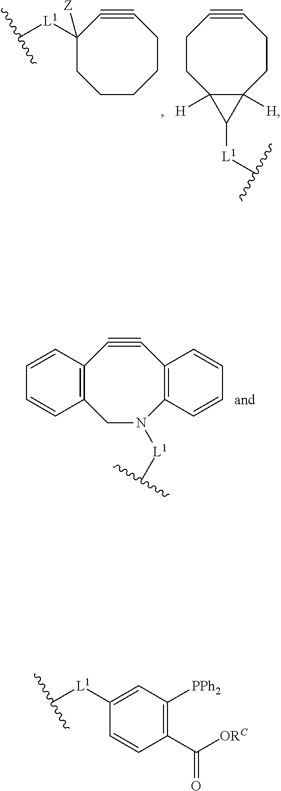
derived from the reactive member reagent;
iii) coupling at least one unit of Formula 2 in said CMC derivative with a compound of Formula B1: 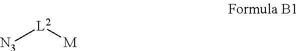
wherein:
L² is an alkylene, -(alkylene)C(O)—*, -(alkylene)NHC(O)—*, —(CH₂CH₂O)ₘCH₂—*, —CH₂(OCH₂CH₂)ₘ—*, arylene, or a bond, wherein the*is the attachment to M and m is an integer between 1 to 450; and
M is a biomolecular unit;
wherein the azide functional group of the compound of Formula B1 reacts with R¹ of the at least one unit of Formula 2 in said CMC derivative via a click reaction to form a CMC derivative product comprising at least one unit of Formula 3: 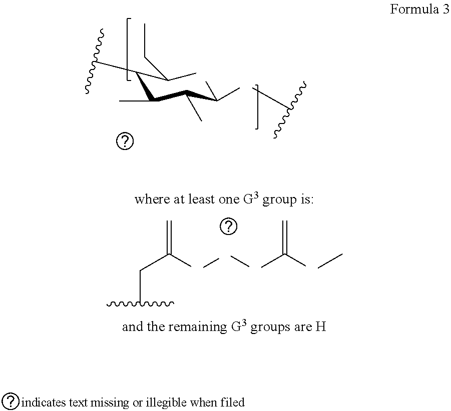
wherein:
X is a linker group selected from: 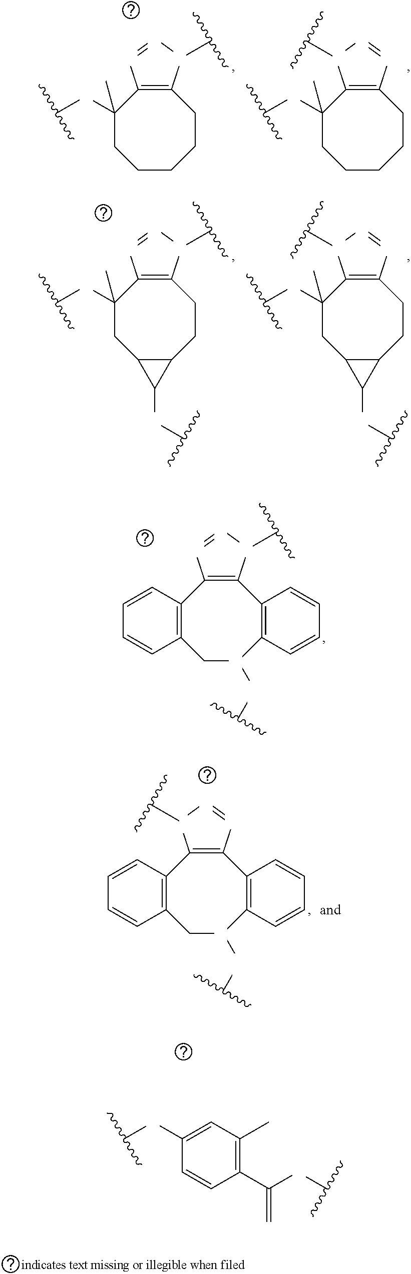
derived from R¹; and
R² is —L²-M derived from the compound of Formula B1.

2. The method of claim 1, wherein step i) comprises the following steps:
i) a) ion exchanging the Na⁺ in Na-CMC with a R₄N⁺ cation to form a CMC derivative comprising at least one unit of Formula lab: 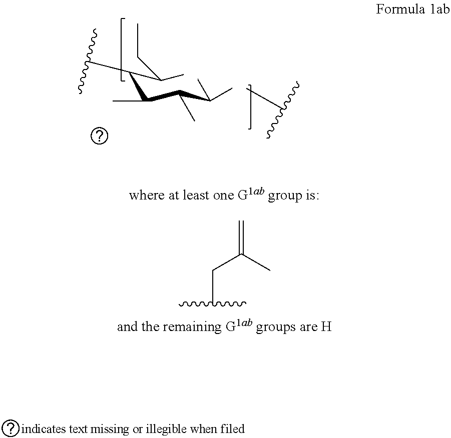
wherein R is alkyl; and
i) c) reacting at least one said unit of Formula 1ab with the spacer to form the CMC derivative comprising at least one unit of Formula 1, wherein the spacer and the CMC derivative comprising at least one unit of Formula 1 are as defined in claim 1.

3. The method of claim 1, wherein step i) comprises the following steps:
i) aa) ion exchanging the Na⁺ in Na-CMC with a H⁺ to form a CMC derivative comprising at least one unit of Formula 1aa: 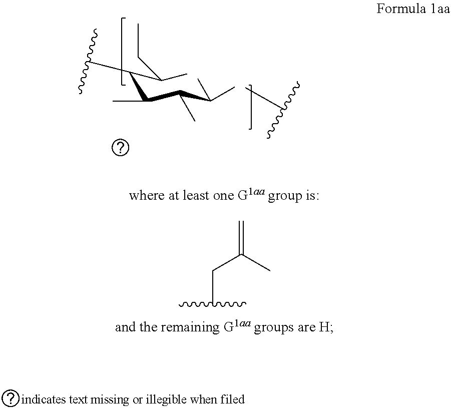
i) ab) ion exchanging the H⁺ of at least one said unit of Formula 1aa with a R₄N⁺ cation to form a CMC derivative comprising at least one unit of Formula 1ab: 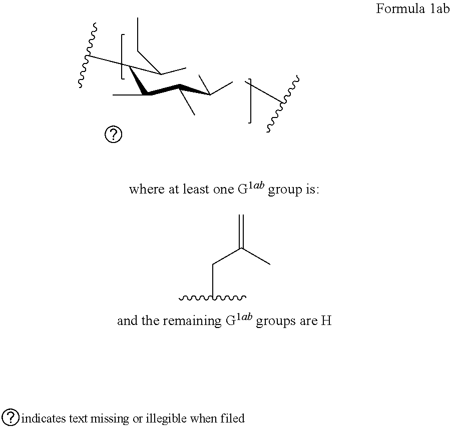
wherein R is alkyl; and
i) c) reacting at least one said unit of Formula lab with the spacer to form the CMC derivative comprising at least one unit of Formula 1, wherein the spacer and the CMC derivative comprising at least one unit of Formula 1 are as defined in claim 1.

4. The method of claim 1, wherein step i) comprises the following steps:
i) b) activating at least one —CH₂COO⁻Na⁺ group of Na-CMC, at least one —CH₂COO⁻H⁺ group of the CMC derivative comprising at least one unit of Formula laa if present, or at least one —CH₂COO⁻R₄N⁺ group of the CMC derivative comprising at least one unit of Formula lab if present, by reacting with an activating reagent to form a CMC derivative comprising at least one unit of Formula 1b: 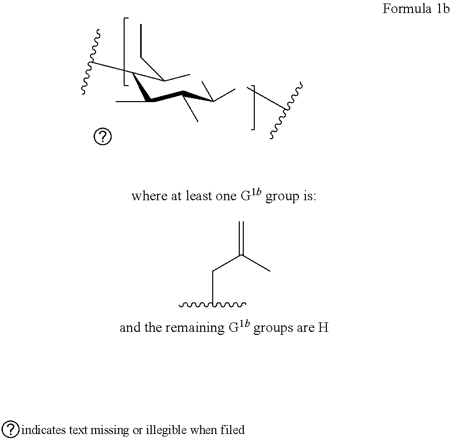
wherein LG is a leaving group; and
i) c) reacting the CMC derivative comprising at least one unit of Formula 1b with the spacer to form the CMC derivative comprising at least one unit of Formula 1, wherein the spacer and the CMC derivative comprising at least one unit of Formula 1 are as defined in claim 1.

5. The method of claim 4, wherein LG is selected from —F, —Cl, —Br, —I, —OR^{LG}, —SR^{LG}, —N⁺R^{LG}₃,
—OC(O)R^{LG} or —OC(O)OR^{LG}, wherein R^{LG} is alkyl, aryl, mesyl, tosyl, triflyl, or

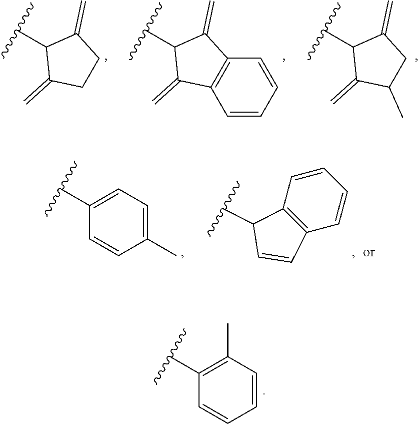

6. The method of claim 1, wherein the degree of substitution (DoS) of units of Formula 1 in the CMC derivative per D-anhydroglucopyranose monomers of cellulose after step i) is at least about 0.25.

7. The method of claim 1, wherein the reactive member reagent is selected from: 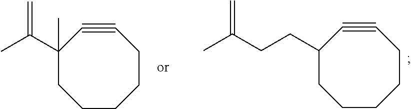
R¹ is selected from: 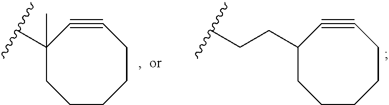
and X is selected from: 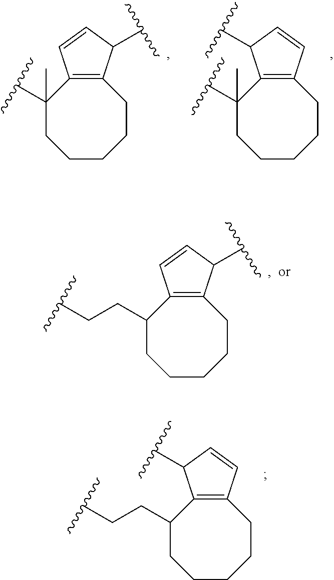 [SMILES: *C1C=CC2=C1CCCCCC2(*)C ; *C1C=CC2=C1C(*)(C)CCCCC2 ; *CCC1CCCCCC2=C1C=CC2* ; *CCC1CCCCCC2=C1C(*)C=C2]
wherein:
LG¹ is —OH or

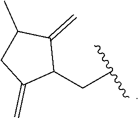 [SMILES: *CC1C(=C)CC(C)C1=C]

8. The method of claim 1, wherein the reactive member reagent is: 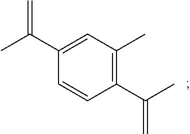
R¹ is: 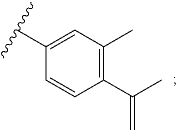 [SMILES: *c1ccc(C(=C)C)c(C)c1]
and X is: 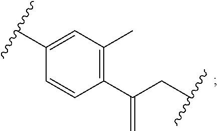 [SMILES: *CC(=C)c1ccc(*)cc1C]
wherein:
LG¹ is —OH or

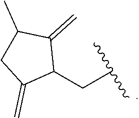 [SMILES: *CC1C(=C)CC(C)C1=C]

9. The method of claim 1, wherein the biomolecular unit M is a saccharide moiety, a metal chelator, a fluorescent probe, or a peptide.

10. The method of claim 9, wherein the saccharide moiety is derived from a saccharide selected from N-acetylglucosamine (GlcNAc), N-acetylmannosamine (ManNAc) and peracetylated N-acetylmannosamine (Man(NAc)Ac₄).

11. The method of claim 9, wherein the metal chelator is a Zn(II) chelator.

12. The method of claim 9, wherein M is: 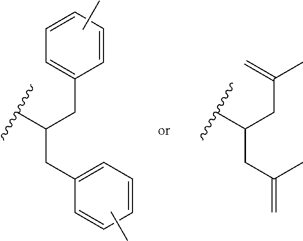
wherein:
y and z are each independently an integer from 0 to 4; and
each R³ and R⁴ is independently selected from —F, —Cl, —Br, and —I, —NO₂, —CO₂H, —CO₂ (alkyl), —CN, alkyl, aryl, and acyl; or, if one or both of y and z is an integer from 2 to 4, two R³ groups or two R⁴ groups on adjacent carbon atoms are linked to form a fused benzene ring.

13. The method of claim 9, wherein M is:

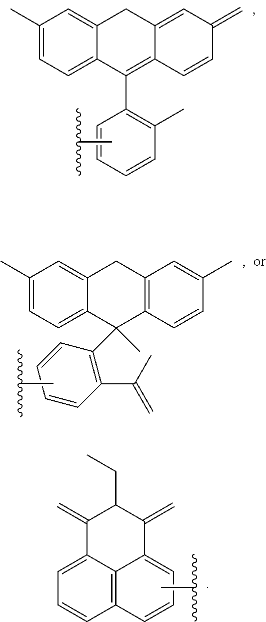

14. The method of claim 9, wherein M is:

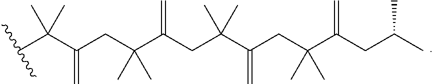

15. A CMC derivative product obtainable by the method of claim 1, wherein the CMC derivative product comprises at least one unit of Formula 3: 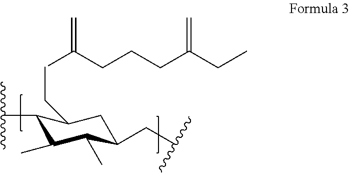
wherein:
L is: 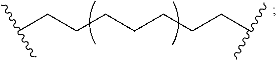
n is an integer from 0 to 450;
X is selected from: 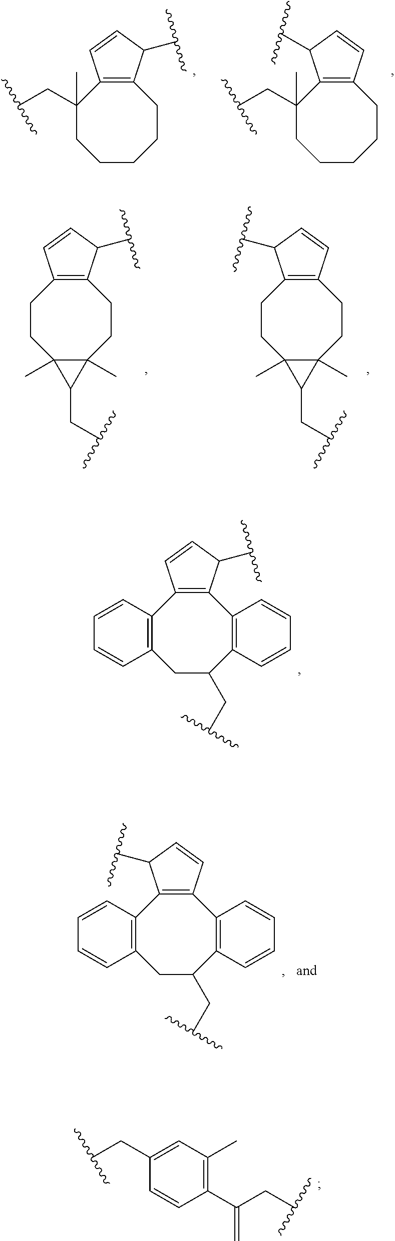
L¹ is a bond, alkylene, -(alkylene)O—*, —O(alkylene)-*, or -(alkylene)C(O)—*;
wherein the*is the attachment to the cyclooctene or benzene ring;
Z is —F, —Cl, —Br, or —I;
R² is —L²-M;
L² is an alkylene, -(alkylene)C(O)—*, -(alkylene)NHC(O)—*, —(CH₂CH₂O)ₘCH₂—*, —CH₂(OCH₂CH₂)ₘ—*, arylene, or a bond, wherein the*is the attachment to M and m is an integer between 1 to 450; and
M is a biomolecular unit.
